SEQUENCE LISTING (42 sequences)

SEQ ID NO: 1 - protein, 40 aa, synthetic construct
DRCERNEFQCQDGKCISYKWVCDGSAECQDGSDESQETCL

SEQ ID NO: 2 - protein, 40 aa, synthetic construct
VTCKSGDFSCGGRVNRCIPQFWRCDGQVDCDNGSDEQGCP

SEQ ID NO: 3 - protein, 38 aa, synthetic construct
KTCSQDEFRCHDGKCISRQFVCDSDRDCLDGSDEASCP

SEQ ID NO: 4 - protein, 40 aa, synthetic construct
LTCGPASFQCNSSTCIPQLWACDNDPDCEDGSDEWPQRCR

SEQ ID NO: 5 - protein, 121 aa, synthetic construct
DRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPV

SEQ ID NO: 6 - protein, 125 aa, synthetic construct
DRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVLTCG

SEQ ID NO: 7 - protein, 163 aa, synthetic construct
DRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVLTCGPASFQCNSSTCIPQLWACDNDPDCEDGSDEWPQRCRGL

SEQ ID NO: 8 - DNA, 366 bp, synthetic construct
  source 1..366: mol_type = other DNA
gacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgtaa

SEQ ID NO: 9 - DNA, 378 bp, synthetic construct
  source 1..378: mol_type = other DNA
gacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggttaa

SEQ ID NO: 10 - DNA, 492 bp, synthetic construct
  source 1..492: mol_type = other DNA
gacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttaa

SEQ ID NO: 11 - protein, 157 aa, synthetic construct
DLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGFTPLHLAAFWGHLEIVEVLLKYGADVNAQDKFGKTPFDLAIDNGNEDIAEVLQKAA

SEQ ID NO: 12 - protein, 106 aa, synthetic construct
QLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSV

SEQ ID NO: 13 - protein, 165 aa, synthetic construct
KEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSSTLGPEKDSRVSVTKPFMLPPVAA

SEQ ID NO: 14 - protein, 165 aa, synthetic construct
EGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAA

SEQ ID NO: 15 - protein, 860 aa, Homo sapiens [sequence omitted]

SEQ ID NO: 16 - protein, 294 aa, synthetic construct
DRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVGSGGGGSDLGKKLLEASRAGQDDEVRILMANGADVNAQDRYGTTPLHLAAWHGHLEIVEVLLKHGADVNANDVKGNTPLHLAANVGHLEIVEVLLKYGADVNAADNWGFTPLHLAAFWGHLEIVEVLLKYGADVNAQDKFGKTPFDLAIDNGNEDIAEVLQKAAGTKHHHHHH

SEQ ID NO: 17 - protein, 304 aa, synthetic construct
DRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVGSGGGGSGGKEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSSTLGPEKDSRVSVTKPFMLPPVAAGTKHHHHHH

SEQ ID NO: 18 - protein, 304 aa, synthetic construct; identical to SEQ ID NO: 17

SEQ ID NO: 19 - protein, 304 aa, synthetic construct
DRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVGSGGGGSGGEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAAGTKHHHHHH

SEQ ID NO: 20 - protein, 9 aa, synthetic construct
GTKHHHHHH

SEQ ID NO: 21 - DNA, 5130 bp, synthetic construct
  source 1..5130: mol_type = other DNA
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattgcggccgtctcaggccgaattcaagcttgccaccatggggatccttcccagccctgggatgcctgcgctgctctccctcgtgagccttctctccgtgctgctgatgggttgcgtagctgaaaccggtgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtcttggtaccaagcaccaccatcaccatcactaatgatcactcgagactagtatcgcgataattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 22 - protein, 172 aa, synthetic construct
DRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVLTCGPASFQCNSSTCIPQLWACDNDPDCEDGSDEWPQRCRGLGTKHHHHHH

SEQ ID NO: 23 - DNA, 495 bp, synthetic construct
  source 1..495: mol_type = other DNA
gacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagggtaccaagcaccaccatcaccatcactaa

SEQ ID NO: 24 - protein, 9 aa, synthetic construct
GSGGGGSGG

SEQ ID NO: 25 - DNA, 6210 bp, synthetic construct
  source 1..6210: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatggggatccttcccagccctgggatgcctgcgctgctctccctcgtgagccttctctccgtgctgctgatgggttgcgtagctgaaaccggtgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgggctctggaggaggtgggagcggcggacagctcactattgaagctgtgccatccaatgctgcagaggggaaggaggttcttctacttgtccacaatctgccccaggaccctcgtggctacaactggtacaaaggggaaacagtggatgccaaccgtcgaattataggatatgtaatatcaaatcaacagattaccccagggcctgcatacagcaatcgagagacaatataccccaatgcatccctgctgatgcggaacgtcaccagaaatgacacaggatcctacaccctacaagtcataaagctaaatcttatgagtgaagaagtaactggccagttcagcgtaggtaccaagcaccaccatcaccatcactaagcggccgctcgagtctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 26 - DNA, 738 bp, synthetic construct
  source 1..738: mol_type = other DNA
gacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgggctctggaggaggtgggagcggcggacagctcactattgaagctgtgccatccaatgctgcagaggggaaggaggttcttctacttgtccacaatctgccccaggaccctcgtggctacaactggtacaaaggggaaacagtggatgccaaccgtcgaattataggatatgtaatatcaaatcaacagattaccccagggcctgcatacagcaatcgagagacaatataccccaatgcatccctgctgatgcggaacgtcaccagaaatgacacaggatcctacaccctacaagtcataaagctaaatcttatgagtgaagaagtaactggccagttcagcgtaggtaccaagcaccaccatcaccatcactaa

SEQ ID NO: 27 - DNA, 711 bp, synthetic construct
  source 1..711: mol_type = other DNA
gacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgggctctggaggaggtgggagcggcggacagctcactattgaagctgtgccatccaatgctgcagaggggaaggaggttcttctacttgtccacaatctgccccaggaccctcgtggctacaactggtacaaaggggaaacagtggatgccaaccgtcgaattataggatatgtaatatcaaatcaacagattaccccagggcctgcatacagcaatcgagagacaatataccccaatgcatccctgctgatgcggaacgtcaccagaaatgacacaggatcctacaccctacaagtcataaagctaaatcttatgagtgaagaagtaactggccagttcagcgtataa

SEQ ID NO: 28 - DNA, 6387 bp, synthetic construct
  source 1..6387: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatggggatccttcccagccctgggatgcctgcgctgctctccctcgtgagccttctctccgtgctgctgatgggttgcgtagctgaaaccggtgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgggctctggaggaggtgggagcggcggaaaggagatctgcgggaatcctgtgactgataatgtaaaagacattacaaaactggtggcaaatcttccaaatgactatatgataaccctcaactatgtcgccgggatggatgttttgcctagtcattgttggctacgagatatggtaatacaattatcactcagcttgactactcttctggacaagttctcaaatatttctgaaggcttgagtaattactccatcatagacaaacttgggaaaatagtggatgacctcgtgttatgcatggaagaaaacgcaccgaagaatataaaagaatctccgaagaggccagaaactagatcctttactcctgaagaattctttagtattttcaatagatccattgatgcctttaaggactttatggtggcatctgacactagtgactgtgtgctctcttcaacattaggtcccgagaaagattccagagtcagtgtcacaaaaccatttatgttaccccctgttgcagccggtaccaagcaccaccatcaccatcactaagcggccgctcgagtctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 29 - DNA, 915 bp, synthetic construct
  source 1..915: mol_type = other DNA
gacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgggctctggaggaggtgggagcggcggaaaggagatctgcgggaatcctgtgactgataatgtaaaagacattacaaaactggtggcaaatcttccaaatgactatatgataaccctcaactatgtcgccgggatggatgttttgcctagtcattgttggctacgagatatggtaatacaattatcactcagcttgactactcttctggacaagttctcaaatatttctgaaggcttgagtaattactccatcatagacaaacttgggaaaatagtggatgacctcgtgttatgcatggaagaaaacgcaccgaagaatataaaagaatctccgaagaggccagaaactagatcctttactcctgaagaattctttagtattttcaatagatccattgatgcctttaaggactttatggtggcatctgacactagtgactgtgtgctctcttcaacattaggtcccgagaaagattccagagtcagtgtcacaaaaccatttatgttaccccctgttgcagccggtaccaagcaccaccatcaccatcactaa

SEQ ID NO: 30 - DNA, 33 bp, synthetic construct
  source 1..33: mol_type = other DNA
ggctcgagaagctagcgccaccatggggatcct

SEQ ID NO: 31 - DNA, 38 bp, synthetic construct
  source 1..38: mol_type = other DNA
cgctctagattatacgctgaactggccagttacttctt

SEQ ID NO: 32 - DNA, 51 bp, synthetic construct
  source 1..51: mol_type = other DNA
cgctctagattacatggctgcccagtgtaggctggagtctccagggggatt

SEQ ID NO: 33 - DNA, 915 bp, synthetic construct
  source 1..915: mol_type = other DNA
gacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgggctctggaggaggtgggagcggcggagaagggatctgcaggaatcgtgtgactaataatgtaaaagacgtcactaaattggtggcaaatcttccaaaagactacatgataaccctcaaatatgtccccgggatggatgttttgccaagtcattgttggataagcgagatggtagtacaattgtcagacagcttgactgatcttctggacaagttttcaaatatttctgaaggcttgagtaattattccatcatagacaaacttgtgaatatagtggatgaccttgtggagtgcgtgaaagaaaactcatctaaggatctaaaaaaatcattcaagagcccagaacccaggctctttactcctgaagaattctttagaatttttaatagatccattgatgccttcaaggactttgtagtggcatctgaaactagtgattgtgtggtttcttcaacattaagtcctgagaaagattccagagtcagtgtcacaaaaccatttatgttaccccctgttgcagccggtaccaagcaccaccatcaccatcactaa

SEQ ID NO: 34 - protein, 7 aa, synthetic construct
GSGGGGS

SEQ ID NO: 35 - DNA, 6338 bp, synthetic construct
  source 1..6338: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatggggattctgccttcacctgggatgcccgccctgctgtcactggtctcactgctgtctgtgctgctgatgggatgcgtcgccgaaacaggcgatcggtgcgagagaaacgagttccagtgccaggacggcaagtgtatctcctacaagtgggtgtgcgatggctctgccgagtgtcaggatggcagcgacgagtcccaggagacatgcctgtctgtgacctgtaagtctggcgacttcagctgcggaggaagggtgaacaggtgtatcccacagttttggcggtgcgatggccaggtggactgtgataatggaagcgacgagcagggatgcccacctaagacctgttcccaggatgagttccggtgccacgacggcaagtgtatctctcggcagttcgtgtgcgacagcgatagagactgtctggatggctccgacgaggcctcttgtcctgtgggaagcggaggaggaggatccgacctgggcaagaagctgctggaggcctccagggcaggacaggacgatgaagtgaggatcctgatggccaacggcgccgatgtgaatgcccaggacagatatggaaccacaccactgcacctggcagcatggcacggacacctggagatcgtggaggtgctgctgaagcacggcgccgatgtgaacgccaatgacgtgaagggaaacacccctctgcatctggcagcaaatgtgggccacctggagattgtcgaagtgctgctgaagtatggagccgatgtgaacgccgccgacaattggggcttcacacctctgcacctggccgccttttggggccacctggagatcgtcgaagtcctgctgaaatatggagctgatgtgaacgctcaggacaagttcggcaagaccccatttgatctggccatcgacaacggcaatgaagatattgctgaagtcctgcagaaggctgctggcacaaaacaccatcatcatcaccactaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 36 - DNA, 885 bp, synthetic construct
  source 1..885: mol_type = other DNA
gatcggtgcgagagaaacgagttccagtgccaggacggcaagtgtatctcctacaagtgggtgtgcgatggctctgccgagtgtcaggatggcagcgacgagtcccaggagacatgcctgtctgtgacctgtaagtctggcgacttcagctgcggaggaagggtgaacaggtgtatcccacagttttggcggtgcgatggccaggtggactgtgataatggaagcgacgagcagggatgcccacctaagacctgttcccaggatgagttccggtgccacgacggcaagtgtatctctcggcagttcgtgtgcgacagcgatagagactgtctggatggctccgacgaggcctcttgtcctgtgggaagcggaggaggaggatccgacctgggcaagaagctgctggaggcctccagggcaggacaggacgatgaagtgaggatcctgatggccaacggcgccgatgtgaatgcccaggacagatatggaaccacaccactgcacctggcagcatggcacggacacctggagatcgtggaggtgctgctgaagcacggcgccgatgtgaacgccaatgacgtgaagggaaacacccctctgcatctggcagcaaatgtgggccacctggagattgtcgaagtgctgctgaagtatggagccgatgtgaacgccgccgacaattggggcttcacacctctgcacctggccgccttttggggccacctggagatcgtcgaagtcctgctgaaatatggagctgatgtgaacgctcaggacaagttcggcaagaccccatttgatctggccatcgacaacggcaatgaagatattgctgaagtcctgcagaaggctgctggcacaaaacaccatcatcatcaccactaa

SEQ ID NO: 37 - protein, 12 aa, synthetic construct
GTIQPYPFSWGY

SEQ ID NO: 38 - protein, 349 aa, Homo sapiens
MGPISAPSCRWRIPWQGLLLTASLFTFWNPPTTAQLTIEAVPSNAAEGKEVLLLVHNLPQDPRGYNWYKGETVDANRRIIGYVISNQQITPGPAYSNRETIYPNASLLMRNVTRNDTGSYTLQVIKLNLMSEEVTGQFSVHPETPKPSISSNNSNPVEDKDAVAFTCEPETQNTTYLWWVNGQSLPVSPRLQLSNGNRTLTLLSVTRNDVGPYECEIQNPASANFSDPVTLNVLYGPDAPTISPSDTYYHAGVNLNLSCHAASNPPSQYSWSVNGTFQQYTQKLFIPNITTKNSGSYACHTTNSATGRNRTTVRMITVSDALVQGSSPGLSARATVSIMIGVLARVALI

SEQ ID NO: 39 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 40 - protein, 151 aa, synthetic construct
GTIQPYPFSWGYGSGGGGSGGDRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVGTKHHHHHH

SEQ ID NO: 41 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 42 - DNA, 507 bp, synthetic construct
  source 1..507: mol_type = other DNA
atgggctggtcctgtatcatcctgttcctggtggccaccgctacaggcgtgcacggcaccatccagccttaccccttcagctggggctacggcagcggcggcggaggatctggcggtgacagatgcgagcggaacgagtttcagtgccaggatggaaaatgcatcagctacaagtgggtgtgcgacggaagcgccgagtgccaggacggctccgatgaatctcaagagacatgtctgtctgtcacctgcaagtccggcgacttctcctgcggcggcagagtgaatcggtgcatccctcagttttggcggtgcgatggccaggtggactgcgacaacggctctgacgaacagggctgccctccaaagacctgttctcaggacgaattcagatgtcacgacggcaagtgcatctccagacagttctgcgacagcgatagagattgcctggacggcagcgacgaggccagctgtcctgtgggcaccaagcaccaccaccaccaccattga

(19) United States
(12) Patent Application Publication
(10) Pub. No.: US 2026/0053948 A1
(43) Pub. Date: Feb. 26, 2026

(54) COMPOSITIONS AND METHODS FOR VIROTHERAPY

(71) Applicant: YEDA RESEARCH AND DEVELOPMENT CO. LTD., Rehovot (IL)

(72) Inventors: Menachem RUBINSTEIN, Tel Aviv (IL); Gideon SCHREIBER, Rehovot (IL); Diana GATAULIN, Rehovot (IL)

(21) Appl. No.: 19/110,403

(22) PCT Filed: Sep. 13, 2023
(86) PCT No.: PCT/IL2023/050993

§ 371 Date: Mar. 10, 2025

Related U.S. Application Data
(60) Provisional application No. 63/444,005, filed on Feb. 8, 2023.

(30) Foreign Application Priority Data
Sep. 15, 2022 (IL) 296547

(51) Int. Cl. A61K 48/00; A61K 35/76; A61K 38/00; A61P 35/00; C07K 14/705; C12N 15/62; C12N 15/86

(52) U.S. Cl. CPC A61K 48/0025; A61K 35/76; A61K 38/00; A61P 35/00; C07K 14/705; C12N 15/62; C12N 15/86; C07K 2319/00; C12N 2740/15043; C12N 2840/002

(57) ABSTRACT

The present invention relates to compositions and methods for producing target-specific therapeutic agents with improved properties, useful in oncolytic viro therapy, gene therapy, cell therapy and immunotherapy. Specifically, the invention in embodiments thereof relates to adapter molecules, capable of directing viruses and viral vectors to specific target cells ex vivo and in vivo, to methods for their preparation and use, and to therapeutic compositions comprising them.

### FIELD OF THE INVENTION

The present invention relates to compositions and methods for providing target-specific therapeutic viral agents with improved properties, useful in oncolytic virotherapy, gene therapy, cell therapy and immunotherapy.

### BACKGROUND OF THE INVENTION

Oncolytic viruses (OV) are replication-competent viruses that have been adapted or developed for the treatment of cancer based on their preferential capacity to infect and kill tumor cells. Accordingly, OV may be defined as non-pathogenic viruses having intrinsic tumor-selective killing activity. Tumor selectivity could be at the level of receptor-mediated cell entry, intracellular antiviral responses and/or restriction factors that determine how susceptible the infected cell is to support viral gene expression and replication, leading to cell death. Attempts to develop improved OV, modified using various molecular biology tools to enhance their compatibility with cancer therapy, have been reported. To date, however, only three OVs are available commercially for the treatment of cancer. These include Rigvir, formerly approved in Latvia and available in Georgia, and Armenia, Oncorine H101 approved in China, and talimogene laherparepvec (T-VEC) approved in the USA. Improved selectivity of OV will broaden their use in cancer therapy.

Vesicular Stomatitis Virus (VSV) is one of the best-studied OV (Cook, J., et al., Blood advances, 2022. 6 (11): p. 3268-3279), but so far, it has not been approved for any clinical indication. VSV is a species of the genus vesiculovirus which belongs to the rhabdovirus family. Vesiculoviruses naturally target farm animals and cause lesions in the mouth and udders, but they are not pathogenic in humans. VSV is considered to be the prototype virus of the genus, while other major serotypes include Cocal virus, VSV New Jersey strain, and Maraba virus. The single-stranded, negative-sense RNA genome of VSV encodes five structural proteins: nucleoprotein, phosphoprotein, matrix protein, glycoprotein, and the viral polymerase. The glycoprotein (VSV-G) dictates receptor recognition, cell entry, and viral fusion to the endosome membrane, thereby delivering its RNA and proteins into the host cell cytoplasm.

VSV uses the low-density lipoprotein (LDL) receptor (LDLR) and its other family members for cell entry (Finkelshtein, D., et al., Proceedings of the National Academy of Sciences, 2013. 110 (18): p. 7306). The ligand-binding domain of all LDLR family members contains multiple class A cysteine-rich repeats, structurally homologous to those of the LDLR. In addition, the EGFP region, which encompasses two EGF-like modules, followed by a series of six YWTD repeats and a third EGF-like module, controls the related processes of lipoprotein release at low pH and recycling of the receptor to the cell surface. Both the ligand-binding domain and the EGFP domain are considered to be required for effective intracellular delivery of the bound ligand (such as LDL or viruses using LDLR for infection such as VSV), In particular, the LDLR beta-propeller domain, which encompasses the YWTD repeats, was reported to mediate endosomal release of the bound ligand, and preventing lysosomal degradation (Davis et al., Nature, 326 (23), 760-765, 1987; Jcon et al., Structure, Vol. 11, 133-136, 2003). Since LDLR and its family members are ubiquitously expressed, VSV and VSV-G-pseudotyped vectors exhibit broad tropism towards many cell types. Hence, upon systemic administration most of the viral particles will be consumed by virus-resistant healthy cells, thereby greatly reducing the effective OV titer.

Various approaches were taken in an attempt to improve the potency and selectivity of VSV towards tumor cells. Many types of tumor cells are defective in their interferon-based antiviral response and therefore are good potential targets for viral oncolysis. Further selectivity of VSV towards tumor cells was obtained by an M51R mutation in the VSV-M protein. This mutation ablated the ability of VSV to attenuate host cell gene expression, including expression of the interferon-induced antiviral proteins. As a result, this M51R-mutation in VSV provided further protection of healthy cells without compromising its oncolytic activity against tumor cells that are defective in their interferon response (Kim, G. N. and C. Y. Kang, Virology, 2007. 357 (1): p. 41-53. Further improvement of specificity was achieved by a recombinant VSV encoding human interferon beta, now undergoing a clinical trial for multiple myeloma (Obuchi et al., Journal of virology, 2003. 77 (16): p. 8843-8856). However, due to the pantropism of VSV, most of the viral particles are taken up and then inactivated by surrounding healthy cells. Hence, there is a room for improving the binding specificity of VSV towards tumor cells.

### Gene Therapy for Monogenic Inherited Disorders

Somatic gene therapy is defined as the addition, removal, or modification of the genetic information in a patient's somatic cells for the purpose of treating or preventing disease. After decades of research and some setbacks, the number of successful and approved gene therapeutics has increased continuously in recent years. To date, gene therapy is used primarily in the therapy of monogenic, mostly rare diseases, and in malignancies.

Viral vectors are the most commonly used tools for inserting the genetic information into the target cells. This process, so-called transduction, can be done ex vivo or in vivo. Viral vectors provide an efficient means for modification of eukaryotic cells, and their use is now commonplace in academic laboratories and industry for both research and clinical gene therapy applications. Over the past two decades, lentiviral vectors (LVVs), derived from the human immunodeficiency virus, have been extensively investigated and optimized. Overall, they are currently the vectors of choice for many gene therapy applications, in particular ex vivo cell therapies, due to their high carrying capacity, ability to be pseudotyped with heterologous envelopes, increased biosafety owing to the generation of self-inactivating vectors, integration into the host genome allowing for sustained transgene expression, and arguably lower immunogenicity of viral proteins. The choice of envelope used to pseudotype LVs dictates various properties of the vector including cell tropism, serum sensitivity, physical, and thermal stability. VSV-G is widely regarded as the “industry standard” envelope to pseudotype LVs owing to its broad tropism, robust stability, and high vector titers.

Third-generation, self-inactivating LVVs have recently been used in multiple clinical trials to introduce genes into hematopoietic stem cells in vitro in order to correct primary immunodeficiencies and hemoglobinopathies. These vectors have also been used to introduce genes into mature T cells to generate immunity to cancer through the delivery of chimeric antigen receptors (CARs) or cloned T-cell receptors. CAR-T-cell therapies, engineered using LVVs, have demonstrated noteworthy clinical success in patients with B-cell malignancies, leading to regulatory approval of the first genetically engineered cellular therapy using LVVs (Milone, M. C. and U. O'Doherty, Leukemia, 2018. 32 (7): p. 1529-1541). LVVs are also being applied in vivo in clinical trials. A phase 1/2 study of a non-primate LVV based upon the equine infectious anemia virus (EIAV), expressing three genes involved in dopamine metabolism demonstrated the safety of local lentiviral gene delivery into the central nervous system with some evidence of clinical benefit. In vivo gene delivery using a LVV has also been applied clinically to the eye for treatment of macular degeneration. In preclinical studies, a LVV phenotypically corrected congenital blood disorders such as beta-thalassemia in utero in a humanized mouse model. So far, there have been 240 LVV-based clinical trials in the US, most of which exploiting VSV-G's advantageous characteristics.

In pre-clinical studies systemic (intravenous) administration of a VSV-G-pseudotyped LVV to mice resulted in transduction mainly of the liver, spleen and the heart (Peng, K. W., et al., Gene Therapy, 2001. 8 (19): p. 1456-1463). In addition, transduction was observed to a lesser extent in other tissues. This lack of target cell specificity highlights the need to develop cell-type-specific vectors for in vivo gene therapy. Several approaches were proposed for targeted cell entry of LVVs (reviewed in: Frank, A. M. and C. J. Buchholz, Molecular Therapy-Methods & Clinical Development, 2019. 12: p. 19-31). In one approach the LVV was co-pseudotyped with two components: a monoclonal antibody directed against a specific cell surface target and a modified VSV-G lacking receptor-binding while maintaining its membrane fusogenic properties. In another approach the lentivector was pseudotyped with a fusion protein of modified Measles H protein and epidermal growth factor (EGF), thereby directing it to cells expressing the EGF receptor. In addition, the H protein was fused to a single-chain antibody directed against CD20 or CD8. Both of these studies require design and production of different pseudotyped LVVs rather than the “industry standard” VSV-G-pseudotyped vector.

Additional approaches for modifying the tropism of gene therapy vectors, in particular lentiviral and adenoviral vectors, have been described. These include for example external bi-specific molecules intended to bind both the viral vector and the target of interest. For example, technologies altering adenoviral vector tropism are discussed in Reetz, et al., (Viruses, 2014. 6 (4): p. 1540-1563), and approaches involving retroviral and lentiviral vectors are suggested in Zhang et al (Retrovirology, 2010. 7:3 (1742-4690 (Electronic)): p. 1-15), Ohno et al. (Biochemical and Molecular Medicine, 1995. 56 (2): p. 172-175), Huckaby, et al., (Journal for Immuno Therapy of Cancer, 2021. 9 (9): p. e002737), Parker et al., (mBio, 2020. 11 (1): p. e02990-19), and U.S. Pat. Nos. 5,753,499 and 5,834,589.

These approaches reportedly encountered various technical challenges, including substantial background transduction levels, large-scale production setbacks low transduction efficacy, and in-vivo immunogenicity associated with rapid serum clearance, and did not result in clinically approved treatments to date. No such approaches for VSV or VSV-G-pseudotyped vectors have been successfully implemented either.

### Respiratory Diseases

Gene therapy is considered as an alternative therapy for respiratory diseases having a genetic origin and no currently approved curative treatment. After five decades of progress, many different vectors and gene editing tools for genetic engineering are now available. However, there is still a long way to achieve a safe and efficient approach to gene therapy application in clinical practice. The most common of such rare respiratory conditions are cystic fibrosis (CF), alpha-1 antitrypsin deficiency (AATD), and primary ciliary dyskinesia (PCD).

CF is an autosomal recessive disease caused by mutations in the cystic fibrosis transmembrane conductance regulator (CFTR) gene, with pediatric onset and a median life expectancy of 37 years. Over 70,000 people are affected by CF in Europe and the United States, and around 2000 different mutations in CFTR gene have been described. The CFTR protein is expressed in epithelial cells located in different organs. Dysfunctional CFTR provokes abnormalities in epithelial electrolyte transport leading to complications in the lungs, intestine, colon, liver, reproductive system, salivary, and sweat glands. Among all of these affected organs and symptoms, it is the respiratory disease that provokes the most severe effects and is the main cause of CF-related death.

The first clinical trials using gene therapy for CF were initiated in the 1990s, shortly after the cloning of the CFTR gene. Subsequently, 27 clinical gene therapy trials involving ˜600 CF patients took place until 2018 without much success. The largest of these was a Phase IIb multiple-dose trial completed by the UK Cystic Fibrosis Gene Therapy Consortium (UK CFGTC) in 2015. This trial of liposome-mediated delivery of a CFTR expression plasmid administered on a monthly basis over the course of a year demonstrated for the first time that gene therapy could provide a modest stabilization in the rate of decline of lung function in CF patients. However, this approach was not sufficient to significantly improve the lung disease (Yan et al., Human molecular genetics, 2019. 28(R1): p. R88-R94).

LVVs have a sufficient packaging capacity for a CFTR expression cassette and also integrate their cargo into the host genome, ensuring persistent expression for the life of the cell. Integration into the host genome also implies that if progenitor cells are transduced, daughter cells expressing the therapeutic transgene can repopulate the surface epithelium. Unlike adenoviral vectors, LVVs can transduce non-dividing cells, a clear advantage for CF gene therapy because most airway epithelial cells are mitotically quiescent. Clinical use of VSV-G-pseudotyped LVVs for gene therapy of cystic fibrosis by inhalation of the vector has not been attempted since the VSV-G receptor (LDLR) is located at the basolateral side of lung epithelial cells and not on their air-exposed apical side (Marquez Loza et al. Genes, 2019. 10 (3): p. 218). Chelating agents and surfactants were developed and utilized in model animals to transiently disrupt the tight junctions in the lung epithelium in order to expose the basolateral LDLR. However, the use of such agents did not improve the transduction efficiency and could increase the risk of infection in the CF lung colonized with pathogenic bacteria Marquez Loza et al., ibid).

Substitution of VSV-G by envelope proteins that are more suitable for inhalation-mediated transduction of lung epithelial cells has been attempted, e.g., Alton et al., Thorax, 2017. 72(2): p. 137; Mitomo et al., Molecular therapy, 2010. 18(6): p. 1173-1182), U.S. Pat. No. 10,704,061. However, such substitutes are not very practical due to poor vector stability, low packaging titer and/or low transduction efficiency compared with the industry standard VSV-G.

### Chimeric Antigen Receptor T-Cell (CAR-T) Immunotherapy

CAR-T cell therapy is a form of adoptive T cell therapy, in which cancer patients receive tumor-specific T cells that were genetically altered and expanded ex vivo. Chimeric antigen receptors (CARs) are typically composed of an extracellular antigen-binding domain, connected via a hinge region and a transmembrane domain to one or more intracellular signaling domains. Upon binding to their targets, CARs induce intracellular signaling that results in antigen-specific killing of the target cell and simultaneous proliferation of the CAR-T cell. This unique therapeutic concept has been implemented in several approved products. Authorizations were granted to Yescarta and Kymriah in the US and Europe, both targeting lymphoma cells via the B cell marker CD19. Two additional products, Tecartus and Abecma, have received regulatory approval, the latter extending indications for CAR therapy to multiple myeloma via B cell maturation antigen (BCMA). Several hundred CAR T cell trials are ongoing worldwide, many of which aim at facilitating manufacturing procedures and addressing additional malignancies. CAR-T cells are especially expensive, since they are individualized cell therapy products requiring time-consuming manufacturing procedures that rely on ex vivo gene transfer into T cells isolated from the patient. Following the isolation of lymphocytes from patients, the cells are activated and subsequently transduced, then expanded and finally re-infused into the patient. This protocol is complex and expensive, resulting in many attempts to simplify it by various strategies, one of which is generation of CAR-T cells in vivo by administration of T-cell-specific vectors to the patient (Michels et al., Molecular Therapy, 2022). Such specificity may potentially be achieved by designing vectors entering cells through a defined cell surface protein present on the T cell.

Designed ankyrin repeat proteins (DARPins) can be selected to become high-affinity binders to any kind of target molecule (Sedgwick and Smerdon, Trends in Biochemical Sciences, 1999. 24 (8): p. 311-316). A DARPin exhibiting high affinity for CD8 was identified by ribosome display selection. It was then incorporated as the outside domain of a trans-membrane protein, replacing VSV-G as means for pseudotyping lentiviral vectors. This vector was effective in transducing CD8⁺ T cells in vivo. Yet, it was not stable enough for large-scale production and clinical use (Frank et al., Human Gene Therapy, 2020. 31 (11-12): p. 679-691).

There remains a need for providing additional and improved compositions and methods for virotherapy. In particular, molecular agents capable of modifying the tropism of viruses and viral vectors, and for improving the selectivity and efficacy of virus-based treatments, would be advantageous.

### SUMMARY OF THE INVENTION

The present invention relates to compositions and methods for producing target-specific therapeutic agents with improved properties, useful in oncolytic virotherapy, gene therapy, cell therapy and immunotherapy. Specifically, the invention in embodiments thereof relates to adapter molecules, capable of directing viruses and viral vectors to specific target cells ex vivo and in vivo, to methods for their preparation and use, and to therapeutic compositions comprising them.

More specifically, disclosed in embodiments of the invention are molecular agents, including fusion proteins and conjugates, that specifically bind and modify the target specificity of viruses and viral vectors comprising the envelope glycoprotein of vesicular stomatitis virus (VSV-G) and homologs thereof. These agents, herein referred to as “adapter molecules” or “adapters”, comprise two covalently bonded components, namely an anchoring component and a targeting component, having unique and advantageous structural and functional properties as described in further detail below.

The invention is based, in part, on experiments performed with newly developed adapter molecules, comprising an anchoring component constructed of cysteine-rich repeat sequences derived from human low-density lipoprotein receptor (LDLR). Surprisingly, the adapter molecules were capable of facilitating VSV-induced oncolysis, as well as gene delivery mediated by VSV-G-decorated lentiviral vectors (LVV), to various target cells, with remarkably high potency and target selectivity. It is further disclosed unexpectedly that the adapters were capable of facilitating effective and selective transduction, despite the absence of certain structural elements and domains hitherto considered to be involved in cell entry, as will be described in further detail below.

Accordingly, provided in embodiments of the invention are adapter molecules, nucleic acid constructs encoding them, viruses and viral vectors comprising the adapters and/or their encoding constructs, pharmaceutical compositions comprising the vectors and/or cells transduced therewith, and methods for their use and manufacture.

In one aspect, the invention relates to an adapter molecule, comprising an anchoring component covalently linked by a flexible linker to a targeting component, wherein:
a. the anchoring component consists essentially of at least one isolated Class A repeat (CR) motif selected from the group consisting of: human low-density lipoprotein receptor (hLDLR) Class-A repeat 2 (hLDLR CR2) and hLDLR CR3, and optionally at least one of hLDLR CR1 and hLDLR CR4.
b. the flexible linker comprises at least four contiguous amino acid residues selected from the group consisting of glycine, serine and/or alanine, and
c. the targeting component comprises a ligand of a receptor expressed preferentially on the surface of a mammalian target cell, or an antigen-binding molecule that selectively binds the receptor.

Thus, hLDLR CR sequences in accordance with embodiments of the invention may include:

(hLDLR CR1, SEQ ID NO: 1)
DRCERNEFQCQDGKCISYKWVCDGSAECQDGSDESQETCL,
(hLDLR CR2, SEQ ID NO: 2)
VICKSGDFSCGGRVNRCIPQFWRCDGQVDCDNGSDEQGCP,
(hLDLR CR3, SEQ ID NO: 3)
KTCSQDEFRCHDGKCISRQFVCDSDRDCLDGSDEASCP,
and/or
(hLDLR CR4, SEQ ID NO: 4)
LTCGPASFQCNSSTCIPQLWACDNDPDCEDGSDEWPQRCR.

In one embodiment, the anchoring component consists essentially of a plurality of the isolated CR motifs. In another embodiment, the anchoring component consists essentially of hLDLR CR1, hLDLR CR2 and hLDLR CR3. In another embodiment, said anchoring component is selected from the group consisting of sLDLR₍₂₅₋₁₄₅₎ (SEQ ID NO: 5), sLDLR₍₂₅₋₁₄₉₎ (SEQ ID NO: 6) and sLDLR₍₂₅₋₁₈₇₎ (SEQ ID NO: 7), as follows:

(SLDLR(25-145), SEQ ID NO: 5)
DRCERNEFQCQDGKCISYKWVCDGSAECQDGSDESQETCLSVTCKSGDFS
CGGRVNRCIPQFWRCDGQVDCDNGSDEQGCPPKTCSQDEFRCHDGKCISR
QFVCDSDRDCLDGSDEASCPV,
(SLDLR(25-149), SEQ ID NO: 6)
DRCERNEFQCQDGKCISYKWVCDGSAECQDGSDESQETCLSVTCKSGDFS
CGGRVNRCIPQFWRCDGQVDCDNGSDEQGCPPKTCSQDEFRCHDGKCISR
QFVCDSDRDCLDGSDEASCPVLTCG,
and
(SLDLR(25-187), SEQ ID NO: 7)
DRCERNEFQCQDGKCISYKWVCDGSAECQDGSDESQETCLSVTCKSGDFS
CGGRVNRCIPQFWRCDGQVDCDNGSDEQGCPPKTCSQDEFRCHDGKCISR
QFVCDSDRDCLDGSDEASCPVLTCGPASFQCNSSTCIPQLWACDNDPDCE
DGSDEWPQRCRGL.

In another embodiment, said receptor is selected from the group consisting of: cluster of differentiation 8 (CD8), CD56, prostate-specific membrane antigen (PSMA), carcinoembryonic antigen-related cell adhesion molecule 6 (CEACAM6), CEACAM1, proto-oncogene c-KIT (c-KIT), and folate receptor (FOLR1). Each possibility represents a separate embodiment of the invention.

In various exemplary embodiments, said targeting component may be selected from the group consisting of:
a) a designed ankyrin repeat protein (DARPin) directed to human CD8, human PSMA, or human CD56;
b) a ligand selected from the group consisting of: human CEACAM8, human SCF, human PSMAL, and fragments thereof comprising at least the receptor binding domain, and
c) an antibody directed to human CEACAM6, human c-KIT or human PSMA, or an antigen-binding fragment thereof.

In certain other exemplary embodiments, said anchoring component consist essentially of sLDLR₍₂₅₋₁₄₅₎ (SEQ ID NO: 5), and said targeting component is selected from the group consisting of: DARPin53F6 (SEQ ID NO: 11), CEACAM8₍₃₅₋₁₄₀₎ (SEQ ID NO: 12), ligand of the prostate-specific membrane antigen (PSMAL), murine pro-KIT-ligand₍₂₆₋₁₉₀₎ (mSCF, SEQ ID NO: 13), and human pro-KIT-ligand₍₂₆₉₋₇₆₃₎ (hSCF, SEQ ID NO: 14). In a particular embodiment, said anchoring component consists essentially of sLDLR₍₂₅₋₁₄₅₎ (SEQ ID NO: 5), and said targeting component is CEACAM8₍₃₅₋₁₄₀₎ (SEQ ID NO: 12). In another particular embodiment, said anchoring component consists essentially of sLDLR₍₂₅₋₁₄₅₎ (SEQ ID NO: 5), and said targeting component DARPin53F6 (SEQ ID NO: 11). In another embodiment, said anchoring component consists essentially of sLDLR₍₂₅₋₁₄₅₎ (SEQ ID NO: 5), and said targeting component consists essentially of a GTI peptide

(GTIQPYPFSWGY, SEQ ID NO: 37).

These exemplary targeting components, including their sequences and exemplified construction and use, are further discussed in the Examples section below. Each possibility represents a separate embodiment of the invention.

In another embodiment, the adapter molecule is a fusion protein consisting essentially of said anchoring component, said targeting component and said linker. In another aspect, there is provided a nucleic acid construct encoding the adapter molecule (in particular, the fusion protein adapters). In another aspect, the invention provides a viral vector comprising the nucleic acid construct. In another embodiment, the invention provides a viral vector comprising the nucleic acid construct, wherein said construct is operably linked to one or more transcription regulation sequences. In various embodiments, the viral vector is selected from the group consisting of a recombinant vesicular stomatitis virus (VSV), Cocal virus (COV), and Maraba virus (Maraba) vectors, wherein each possibility represents a separate embodiment of the invention.

In another aspect, there is provided an adapter molecule, comprising an anchoring component covalently linked by a flexible linker to a targeting component, wherein:
a. the anchoring component consists essentially of at least one isolated CR motif selected from the group consisting of: hLDLR CR2 and hLDLR CR3, and optionally at least one of hLDLR CR1 and hLDLR CR4, and
b. the targeting component comprises a ligand of a receptor expressed preferentially on the surface of a mammalian target cell, said ligand selected from a selective PSMA ligand (PSMAL) comprising a Glu-NH—CO—NH-Lys pharmacophore, and folic acid.

In another embodiment, the anchoring component consists essentially of a plurality of the isolated CR motifs. In another embodiment, the anchoring component consists essentially of hLDLR CR1, hLDLR CR2 and hLDLR CR3. In another embodiment, said anchoring component is selected from the group consisting of: sLDLR₍₂₅₋₁₄₅₎ (SEQ ID NO: 5), sLDLR₂₅₋₁₄₉ (SEQ ID NO: 6) and sLDLR₂₅₋₁₈₇ (SEQ ID NO: 7). In another embodiment, said adapter molecule consists essentially of sLDLR₂₅₋₁₄₉ (SEQ ID NO: 6) chemically conjugated to (7S,14S,18S)-7-amino-1-(3-(2,5-dioxo-2,5-dihydro-1H-pyrrol-1-yl)phenyl)-1,8,16-trioxo-2,9,15,17-tetraazaicosane-14,18,20-tricarboxylic acid. In another embodiment, said adapter molecule consists essentially of sLDLR₍₂₅₋₁₄₅₎ (SEQ ID NO: 5) chemically conjugated to folic acid via a flexible poly(oxyethylene) linker.

In another embodiment, an adapter molecule of the invention is specifically complexed in a non-covalent manner with particles of a virus or viral vector decorated with a vesiculovirus envelope glycoprotein (G) selected from the group consisting of: VSV-G, COV-G Maraba-G, to form adapter-modified viral particles.

In another aspect, there is provided a pharmaceutical composition comprising a therapeutically effective amount of the adapter-modified viral particles, further comprising a pharmaceutically acceptable carrier, excipient or diluent. In another embodiment, the pharmaceutical composition further comprises at least one of: (i) a proprotein convertase subtilisin/kexin type-9 (PCSK9) polypeptide, and (ii) a second composition of said adapter molecule, such that the total amount of said adapter molecule in said pharmaceutical composition is in excess of said viral particles. Each possibility represents a separate embodiment of the invention.

In another aspect, the adapter molecule as disclosed herein is for use in delivering a virus or viral vector selectively into a target cell in a subject in need thereof, wherein the use comprises contacting particles of the virus or viral vector with said adapter molecule so as to produce adapter-modified viral particles, and administering the resulting adapter-modified viral particles to the subject. In various embodiments, said virus or viral vector is selected from the group consisting of: VSV, COV, Maraba, and viral vectors derived from vesiculovirus, retrovirus and lentivirus strains. In another embodiment, said target cell is selected from the group consisting of: a tumor cell, an immune cell, a hematopoietic stem cell (HSC), and a lung epithelial cell. In another embodiment, said target cell is a tumor cell and said receptor is selected from the group consisting of: PSMA, c-KIT, FOLR1, CEACAM6 and CEACAM1. In another embodiment said target cell is an immune cell and said receptor is CD8 or CD56. In another embodiment said target cell is a lung epithelial cell and said receptor is CEACAM6 or CEACAM1. In another embodiment, said target cell is a HSC and said receptor is FOLR1.

In another aspect, there is provided a pharmaceutical composition, comprising a therapeutically effective amount of adapter-modified viral particles and a pharmaceutically acceptable carrier, excipient or diluent, the particles comprising:
(i) an adapter molecule, comprising an anchoring component covalently linked by a flexible linker to a targeting component, wherein:
   a. the anchoring component comprises: at least one isolated CR motif selected from the group consisting of: hLDLR CR2, hLDLR CR3, and homologs thereof,
   b. the flexible linker comprises at least five contiguous amino acid residues selected from the group consisting of glycine, serine and/or alanine,
   c. the targeting component comprises: a ligand of a receptor expressed preferentially on the surface of a mammalian target cell, or an antigen-binding molecule that selectively binds the receptor, and
(ii) particles of a virus or viral vector decorated with a vesiculovirus envelope glycoprotein (G) selected from the group consisting of: vesicular stomatitis virus (VSV)-G, Cocal virus (COV)-G and Maraba virus (Maraba)-G,
wherein the vesiculovirus envelope glycoprotein is specifically complexed in a non-covalent manner with the anchoring component of the adapter molecule of (i), and
wherein the composition further comprises at least one of: (iii) a PCSK9 polypeptide, and (iv) said adapter molecule at an additional amount in excess of said viral particles.

In one embodiment, the pharmaceutical composition comprises the PCSK9 polypeptide of (iii) and the adapter molecule of (iv). In another embodiment, the pharmaceutical composition comprises the PCSK9 polypeptide of (iii) at an amount effective to provide a blood concentration of 0.01-0.1 μM upon administration to a subject in need thereof, and/or the adapter molecule of (iv) at an amount effective to provide a blood concentration of 1-10 μg/mL upon administration to a subject in need thereof. In another embodiment, the pharmaceutical composition comprises the PCSK9 polypeptide and the effective amount is 5-500 mg. In another embodiment the pharmaceutical composition comprises the adapter molecule of (iv) and the effective amount is 60-600 μg. Each possibility represents a separate embodiment of the invention.

In another embodiment of the pharmaceutical compositions disclosed herein, the anchoring component of said adapter consists essentially of a plurality of the isolated CR motifs. In another embodiment, said anchoring component consists essentially of hLDLR CR1, hLDLR CR2 and hLDLR CR3. In another embodiment, the homolog CR motif is derived from a member of the LDLR family selected from the group consisting of LDLR, very-low-density-lipoprotein receptor (VLDLR). Low-density lipoprotein receptor-related protein 8 (LRP8, ApoER2), Low density lipoprotein receptor-related protein 4 (LRP4), LDLR-related protein 1 (LRP1), LDLR-related protein 1b (LRP1b), and Megalin, and retains the ability to be complexed with the vesiculovirus envelope glycoprotein under physiological conditions. In another embodiment, said member of the LDLR family is selected from the group consisting of human LDLR, LRP1, LRP8 and VLDLR receptors and said composition comprises an effective amount of said PCSK9 polypeptide. Each possibility represents a separate embodiment of the invention. Typically, said anchoring component binds reversibly to VSV-G.

In another embodiment, said receptor is selected from the group consisting of: CD8, Carcinoembryonic Antigen-related Cell Adhesion Molecule 1 (CEACAM1), CEACAM6, c-KIT, PSMA, CD56, Epidermal Growth Factor receptor (EGFR), Human Epidermal growth factor Receptor 2 (HER2), CEA, Emmprin, Endoglin, Epithelial Cellular Adhesion Molecule (EpCAM), Folate Receptor, Glucose-Regulated Protein 78 (GRP 78), Insulin-like Growth Factor 1 Receptor (IGF-1R), Mesothelin, Mucin 1 (Muc-1), and Prostate Stem Cell Antigen (PSCA). In another embodiment said receptor is selected from the group consisting of: CD8, CEACAM1, CEACAM6, c-KIT. PSMA, CD56, and FOLR1. In another embodiment said receptor is selected from the group consisting of: CD8, CEACAM1, CEACAM6, c-KIT. PSMA, and FOLR1. In another embodiment said receptor is a tumor-associated antigen (TAA). Each possibility represents a separate embodiment of the invention.

In another embodiment, said viral vector further encodes a chimeric antigen receptor (CAR), a gene therapy agent (GTA) or a gene editing agent. In another embodiment, the targeting component comprises an antigen-binding molecule that selectively binds to human CD8 or CD56, and said viral vector is a lentiviral or retroviral vector pseudotyped with said envelope glycoprotein, and encoding a CAR directed to a tumor-associated antigen (TAA). In another embodiment, the targeting component comprises an antigen-binding molecule that selectively binds to human CD8, and said viral vector is a lentiviral or retroviral vector pseudotyped with said envelope glycoprotein, and encoding a CAR directed to a TAA. In another embodiment, said GTA is selected from the group consisting of: Cystic Fibrosis Transmembrane Conductance Regulator (CFTR), Adenosine Deaminase, Survival of Motor Neuron 1 (SMN1), Hemoglobin subunit beta, ABCD1. Aryl sulfatase A, and ARPC1B gene products. In a particular embodiment, said GTA is a CFTR gene product. Each possibility represents a separate embodiment of the invention.

In another embodiment said GTA is a human CFTR (hCFTR) gene product, said viral vector is a lentiviral or retroviral vector pseudotyped with said envelope glycoprotein and the targeting component of said adapter molecule comprises a CEACAM6- or CEACAM1-binding portion of human CEACAM8 (hCEACAM8), or an antigen-binding portion of an antibody directed to human CEACAM6 (hCEACAM6) or human CEACAM1 (hCEACAM1). In another embodiment, said GTA is human CFTR, said viral vector is a lentiviral or retroviral vector pseudotyped with said envelope glycoprotein and the targeting component of said adapter molecule comprises a CEACAM1-binding portion of hCEACAM5. In another embodiment said adapter molecule is characterized in that said anchoring component consists essentially of sLDLR₍₂₅₋₁₄₅₎ (SEQ ID NO: 5), and said targeting component is CEACAM8₍₃₅₋₁₄₀₎ (SEQ ID NO: 12).

In another embodiment, said virus is an oncolytic virus (OV) further encoding said adapter protein. In another embodiment, the OV is a vesiculovirus encoding said envelope glycoprotein, and the targeting component of said adapter molecule is directed to a TAA. In another embodiment the TAA is selected from the group consisting of human CEACAM6, human CEACAM1, human c-KIT, and human PSMA, wherein each possibility represents a separate embodiment of the invention.

In another aspect, there is provided a process for producing the pharmaceutical composition, comprising contacting the particles of the virus or viral vector of (ii) as disclosed herein with adapter molecule of (i) as disclosed herein, so as to produce the adapter-modified viral particles.

In another embodiment, the contacting is performed in vitro, by incubating said particles with said adapter molecules under conditions so as to allow specific non-covalent complexing of said particles with the anchoring component of said adapter molecule. In another embodiment, said particles and said adapter molecule are expressed in a mammalian expression system and said contacting is performed in said expression system. In another embodiment, the process further comprises admixing the particles of (ii) or the adapter-modified viral particles with said adapter molecule of (iv), so as to produce a pharmaceutical composition comprising said adapter-modified viral particles and an excess of adapter molecules that are not complexed with said viral particles. Additionally or alternatively, said process further comprises admixing the particles of (ii) or the adapter-modified viral particles with said PCSK9 polypeptide of (iii).

In another embodiment, the pharmaceutical composition as disclosed herein is for use in treating a disease or condition in a subject in need thereof. In various embodiments, the disease or condition is selected from the group consisting of a tumor, an inherited monogenic disorder, and a genetic respiratory condition. Each possibility represents a separate embodiment of the invention.

In another embodiment, the disease or condition is a tumor, and the composition is characterized in that said viral vector further encodes a CAR, a GTA or a gene editing agent, optionally wherein the targeting component comprises an antigen-binding molecule that selectively binds to human CD8 or CD56, and said viral vector is a lentiviral or retroviral vector pseudotyped with said envelope glycoprotein, and encoding a CAR directed to a TAA.

In another embodiment, the disease or condition is a tumor, and the composition is characterized in that said virus is an OV further encoding said adapter protein. In another embodiment, the disease or condition is a tumor, and the composition is characterized in that the OV is a vesiculovirus encoding said envelope glycoprotein, and the targeting component of said adapter molecule is directed to a TAA. In various embodiments, the TAA is selected from the group consisting of human CEACAM6, human CEACAM1, human c-KIT, and human PSMA. Each possibility represents a separate embodiment of the invention. In other embodiments, said tumor is selected from the group consisting of a hematological tumor, a lung tumor, a prostate tumor, a breast tumor, a gynecological tumor, a pancreatic tumor and malignant glioma, wherein each possibility represents a separate embodiment of the invention.

In another embodiment, the targeting component of said adapter molecule comprises an antigen-binding molecule that selectively binds to human CD8 or CD56, and said viral vector is a lentiviral or retroviral vector pseudotyped with said envelope glycoprotein, and encoding a CAR directed to a TAA. In another embodiment the use comprises administration of said composition to said subject to thereby generate tumor-specific immune cells in vivo. In another embodiment the use comprises incubating immune cells of a subject with said composition ex vivo to thereby generate tumor-specific immune cells, and re-introducing the resulting immune cells to said subject. In a particular embodiment, said adapter molecule is characterized in that said anchoring component consist essentially of sLDLR₍₂₅₋₁₄₅₎ (SEQ ID NO: 5), and said targeting component is DARPin53F6 (SEQ ID NO: 11).

In another embodiment said virus is an OV further encoding said adapter protein, and said adapter molecule is characterized in that said anchoring component consist essentially of sLDLR₍₂₅₋₁₄₅₎ (SEQ ID NO: 5), and said targeting component is selected from the group consisting of: CEACAM8₍₃₅₋₁₄₀₎ (SEQ ID NO: 12), PSMAL, and hSCF (SEQ ID NO: 14). In another embodiment said virus is an OV further encoding said adapter molecule, and said adapter molecule is characterized in that said anchoring component consists essentially of sLDLR₍₂₅₋₁₄₅₎ (SEQ ID NO: 5), and said targeting component is CEACAM8₍₃₅₋₁₄₀₎ (SEQ ID NO: 12).

In another embodiment the disease or condition is an inherited monogenic disorder. In another embodiment said disorder is cystic fibrosis. In another embodiment the disease or condition is cystic fibrosis, and said GTA is human CFTR, said viral vector is a lentiviral or retroviral vector pseudotyped with said envelope glycoprotein and the targeting component of said adapter molecule comprises a receptor-binding portion of hCEACAM8, a receptor-binding portion of hCEACAM5, or an antigen-binding portion of an antibody directed to hCEACAM6. In another embodiment said adapter molecule is characterized in that said anchoring component consist essentially of sLDLR₍₂₅₋₁₄₅₎ (SEQ ID NO: 5), and said targeting component is CEACAM8₍₃₅₋₁₄₀₎ (SEQ ID NO: 12).

In another embodiment in the pharmaceutical compositions for use as disclosed herein, the use further comprises administering to said subject a PCSK9 polypeptide prior to and/or concomitantly with administration of said pharmaceutical composition. In a particular embodiment, the PCSK9 polypeptide is administered at a total dose of 5-500 mg per subject over a time period of 1-5 hours initiated at least one hour prior to administration of said pharmaceutical composition and maintained until administration of said pharmaceutical composition is completed. In another embodiment, the use further comprises administering to said subject a second pharmaceutical composition comprising said adapter molecule that is not complexed with viral particles at an effective amount of 60-600 μg.

In another aspect, there is provided a method of delivering a virus or viral vector selectively into a target cell in a subject in need thereof, comprising contacting particles of the virus or viral vector with the adapter molecule as disclosed herein, so as to produce adapter-modified viral particles, and administering the resulting adapter-modified viral particles to the subject. In another embodiment, said virus is selected from the group consisting of VSV, COV and Maraba viruses, or wherein said viral vector is selected from the group consisting of vesiculoviral and lentiviral vectors. In another embodiment, said target cell is selected from the group consisting of: a tumor cell, an immune cell, a HSC, and a lung epithelial cell.

In another embodiment, the method is characterized by one of the following:
a) said target cell is a tumor cell and said receptor is selected from the group consisting of: PSMA, c-KIT, FOLR1, CEACAM6 and CEACAM1;
b) said target cell is an immune cell and said receptor is CD8 or CD56;
c) said target cell is a lung epithelial cell and said receptor is CEACAM6 or CEACAM1; or
d) said target cell is a HSC and said receptor is FOLR1.

In another embodiment, the method is characterized by one of the following:
a) said target cell is a PSMA+ tumor cell, said targeting component of said adapter is a selective PSMA ligand (PSMAL) comprising a Glu-NH—CO—NH-Lys pharmacophore or a GTI peptide (GTIQPYPFSWGY, SEQ ID NO: 37), and said virus or viral vector is an oncolytic vesiculovirus or a vesiculoviral vector further encoding said adapter molecule;
c) said target cell is a CD8+ immune cell, said targeting component of said adapter is DARPin53F6 (SEQ ID NO: 11), and said viral vector is a VSV-G pseudotyped lentiviral or retroviral vector encoding a CAR directed to a TAA;
d) said target cell is a lung epithelial cell, said targeting component of said adapter is CEACAM8₍₃₅₋₁₄₀₎ (SEQ ID NO: 12), and said viral vector is a VSV-G pseudotyped lentiviral or retroviral vector encoding a CFTR gene product; or
e) said target cell is a HSC, said targeting component of said adapter is folic acid, and said viral vector is a VSV-G pseudotyped lentiviral or retroviral vector encoding a GTA.

In another embodiment, said GTA is selected from the group consisting of ADA, Hemoglobin subunit beta, ABCD1, Aryl sulfatase A, ARPC1B, IL2RG, WAS, CYBB, CD18, DCLRE1C, FANCA, PKLR, IDUA, CTNS, and GLA gene products, wherein each possibility represents a separate embodiment of the invention.

In another aspect, the invention provides a method of treating a disease or condition in a subject in need thereof, comprising administering to the subject the pharmaceutical composition as disclosed herein. In another embodiment the disease or condition is an inherited monogenic disorder, and said composition comprises adapter-modified particles of a viral vector encoding a GTA. In another embodiment the disorder is cystic fibrosis, and the GTA is a hCFTR gene product. In another embodiment, said viral vector is a lentiviral or retroviral vector pseudotyped with said envelope glycoprotein and the targeting component of said adapter molecule comprises a receptor-binding portion of hCEACAM8, a receptor-binding portion of hCEACAM5, or an antigen-binding portion of an antibody directed to hCEACAM6. In another embodiment said adapter molecule is characterized in that said anchoring component consists essentially of sLDLR₍₂₅₋₁₄₅₎ (SEQ ID NO: 5), and said targeting component is CEACAM8₍₃₅₋₁₄₀₎ (SEQ ID NO: 12).

In another embodiment the disease or condition is a tumor. In another embodiment the tumor is selected from the group consisting of a hematological tumor, a lung tumor, a prostate tumor, a breast tumor, a gynecological tumor, a pancreatic tumor and malignant glioma, wherein Each possibility represents a separate embodiment of the invention. In another embodiment said virus is an OV further encoding said adapter molecule. In another embodiment said adapter molecule is characterized in that said anchoring component consists essentially of sLDLR₍₂₅₋₁₄₅₎ (SEQ ID NO: 5), and said targeting component is CEACAM8₍₃₅₋₁₄₀₎ (SEQ ID NO: 12). In another embodiment said tumor is a PSMA⁺ prostate tumor, and said targeting component of said adapter is a selective PSMA ligand (PSMAL) comprising a Glu-NH—CO—NH-Lys pharmacophore or a GTI peptide (GTIQPYPFSWGY, SEQ ID NO: 37). In another embodiment the targeting component of said adapter molecule comprises an antigen-binding molecule that selectively binds to human CD8 or CD56, and said viral vector is a lentiviral or retroviral vector pseudotyped with said envelope glycoprotein, and encoding a CAR directed to a TAA on said tumor. In another embodiment the method comprises administering said composition to said subject to thereby generate tumor-specific immune cells in vivo, or comprises incubating immune cells of a subject with said composition ex vivo to thereby generate tumor-specific immune cells, and re-introducing the resulting immune cells to said subject.

In another embodiment said adapter molecule is characterized in that said anchoring component consist essentially of sLDLR₍₂₅₋₁₄₅₎ (SEQ ID NO: 5), and said targeting component is DARPin53F6 (SEQ ID NO: 11). In another embodiment, said anchoring component in the methods of the invention is selected from the group consisting of: sLDLR₍₂₅₋₁₄₅₎ (SEQ ID NO: 5), sLDLR₍₂₅₋₁₄₉₎ (SEQ ID NO: 6) and sLDLR₍₂₅₋₁₈₇₎ (SEQ ID NO: 7).

In another embodiment, the methods of the invention further comprise administering to said subject a PCSK9 polypeptide prior to and/or concomitantly with administration of said pharmaceutical composition, at a total dose of 5-500 mg per subject over a time period of 1-5 hours initiated at least one hour prior to administration of said pharmaceutical composition and maintained until administration of said pharmaceutical composition is completed, and/or further comprise administering to said subject a second pharmaceutical composition comprising said adapter molecule that is not complexed with viral particles at an effective amount of 60-600 μg.

Other objects, features and advantages of the present invention will become clear from the following description and drawings.

### BRIEF DESCRIPTION OF THE DRAWINGS

FIG. 1. Map of pHLsec vector encoding sLDLR₍₂₅₋₁₈₇₎ (sLDLRpHLsec).

FIG. 2. SDS-PAGE of affinity-purified sLDLR₍₂₅₋₁₈₇₎.

FIG. 3. Neutralization of VSV cytopathic effect of Hela cells with sLDLR₍₂₅₋₁₈₇₎.

FIG. 4. Map of pcDNA3.1 vector encoding sLDLR-CEACAM8.

FIG. 5. SDS-PAGE of affinity-purified sLDLR-CEACAM8.

FIG. 6. Neutralization of VSV cytopathic effect of HeLa cells with sLDLR-CEACAM8.

FIGS. 7A-7B. Selective lysis of CaCo2 cells by VSV in the presence of sLDLR-CEACAM8. FIG. 7A—HeLa cells, FIG. 7B—CaCo2 cells.

FIGS. 8A-8B. Selective lysis of AsPC-1 cells by VSV in the presence of sLDLR-CEACAM8. FIG. 8A—HeLa cells, FIG. 8B—AsPC-1 cells.

FIGS. 9A-9B. Inhibition of HeLa cell transduction by LVV using sLDLR-CEACAM8. FIG. 9A—immunohistochemistry staining for CEACAM6, FIG. 9B—relative fluorescence intensity of Hela cells transduced with a VSV-G-pseudotyped, eGFP-encoding lentiviral vector (LVV) in the presence or absence of adapters.

FIGS. 10A-B. Selective transduction of CaCo2 cells by LVV in the presence of sLDLR-CEACAM8. FIG. 10A—immunohistochemistry staining for CEACAM6, FIG. 10B—relative fluorescence intensity of CaCo2 cells transduced with a VSV-G-pseudotyped, eGFP-encoding LVV in the presence or absence of adapters.

FIG. 11. Map of a pcDNA3.1 vector encoding sLDLR-SCF.

FIG. 12. SDS-PAGE of affinity-purified sLDLR-SCF.

FIG. 13. Neutralization of VSV cytopathic effect in Hela cells with sLDLR-SCF.

FIG. 14. Preparation of sLDLR-PSMAL.

FIG. 15. Map of pcDNA3.1 vector encoding sLDLR-DARPin53F6.

FIG. 16. SDS-PAGE of purified sLDLR-53F6.

FIG. 17. Neutralization of VSV cytopathic effect of HeLa cells with sLDLR₍₂₅₋₁₈₇₎ and sLDLR-DARPin53F6.

FIG. 18. Transduction efficiency of MOLT4 cells with EGFP-encoding VSV-G-pseudotyped LVV in the presence of sLDLR or sLDLR-DARPin56F3.

FIG. 19. SDS-PAGE of affinity-purified GTI-sLDLR.

FIG. 20. GTI-sLDLR and sLDLR effectively inhibit VSV infectivity in PSMA-negative prostate cancer PC3 cells.

FIG. 21. GTI-sLDLR allows selective VSV infectivity of PSMA-expressing LNCaP cells.

### DETAILED DESCRIPTION OF THE INVENTION

The present invention relates to compositions and methods for providing target-specific therapeutic viral agents with improved properties, useful in oncolytic virotherapy, gene therapy, cell therapy and immunotherapy.

The present invention in embodiments thereof provides agents termed “adapters”, comprising or consisting of two covalently bonded components. One component is a ligand-binding domain derived from a cellular receptor capable of reversibly binding to the envelope glycoprotein of vesicular stomatitis virus (VSV-G) or to the envelope proteins of Cocal virus (Cocal-G), or that of Maraba virus (Maraba-G). Covalently-bound to it is a second component that serves as a ligand of a specific cell surface receptor or receptor-associated protein, capable of eliciting endocytosis of the ligand-receptor complex. Alternatively, the second component is an antibody or antibody domain, directed against said cell surface receptor, and capable of eliciting endocytosis of the antibody-receptor complex. Another alternative is a Designed Ankyrin Repeat Protein (DARPin) or LoopDARPin, selected for high-affinity binding to a specific cell-surface receptor or receptor-associated protein. Said adapters may be used for improving gene therapy, for viral oncolysis, and for generating CAR-T and CAR-NK cells. Binding of such adapters to viruses, viral vectors, or other vectors carrying said viral envelope proteins, improve their selectivity towards specific cell types, both in vivo and in vitro. According to advantageous embodiments of the invention, further selectivity is obtained by administration of proprotein convertase subtilisin/kexin type-9 (PCSK9) under conditions so as to reduce cell surface levels of LDLR and other LDLR family members prior to and during infusion of said adapter-vector complexes to a patient in need.

Viruses and other vectors decorated with VSV-G, Cocal-G or Maraba-G are pantropic. Hence, following administration to a patient in need they are typically consumed by all cell types and not only by the desired target cells. In contrast, administration of said viruses and vectors coated with adapter molecules of the invention as disclosed herein may reduce vector loss by directing these viruses and vectors to specific cell types. It is further disclosed herein, that the use of adapters in accordance with embodiments of the invention provide for maintaining the advantages of using the “industry standard” VSV-G-pseudotyped vectors, including, but not limited to, safety and stability. In addition, adapters in accordance with embodiments of the invention consisting of human sequences or domains provide for reduced immunogenicity, thereby reducing the risk of anti-VSV-G antibody formation that may impair treatment efficacy.

As disclosed herein, adapter molecules of the invention were unexpectedly determined to mediate highly selective and potent viral therapy using short anchoring sequences derived from C′-truncated human LDLR, which notably lack the LDLR beta-propeller domain (amino acid residues 396-664 of human pro-LDLR), hitherto considered to be required for ligand endocytosis by facilitating endosomal release. In other words, while the beta-propeller domain was considered to mediate reversible binding of LDLR to LDLR-targeting viruses, thereby facilitating effective infection, the present invention unexpectedly demonstrates highly effective infection by viral particles complexed with adapters of the invention, in the absence of the beta propeller region and its characteristic YWTD repeat motifs.

The advantageous properties of adapter molecules in accordance with the principles of the invention were exemplified in various experimental systems. In particular, disclosed herein is the construction of adapters targeting tumor-associated antigens (TAA) such as PSMA, carcinoembryonic antigen-related cell adhesion molecule 6 (CEACAM6), CEACAM1, proto-oncogene c-KIT (c-KIT) and folate receptor (FOLR1), as well as cell surface receptors expressed preferentially on immune cells (such as CD8, expressed on cytotoxic lymphocytes) or on G-CSF-activated hematopoietic stem cells (HSC, such as FOLR1). The adapters were demonstrated to mediate VSV-induced oncolysis, as well as gene delivery mediated by VSV-G-decorated lentiviral vectors (LVV), with unexpectedly high potency and target selectivity.

In one aspect, there is provided an adapter molecule, comprising an anchoring component covalently linked by a flexible linker to a targeting component, wherein the anchoring component consists essentially of at least one isolated Class A repeat (CR) motif selected from the group consisting of: human low-density lipoprotein receptor (hLDLR) Class-A repeat 2 (hLDLR CR2) and hLDLR CR3, and optionally at least one of hLDLR CR1 and hLDLR CR4, and the targeting component comprises a ligand of a receptor expressed preferentially on the surface of a mammalian target cell, or an antigen-binding molecule that selectively binds the receptor. In one embodiment, the anchoring component consists essentially of a plurality of the isolated CR motifs. In another embodiment, the anchoring component consists essentially of hLDLR CR1, hLDLR CR2 and hLDLR CR3. In another embodiment, said anchoring component is selected from the group consisting of: sLDLR₍₂₅₋₁₄₅₎ (SEQ ID NO: 5), sLDLR₍₂₅₋₁₄₉₎ (SEQ ID NO: 6) and sLDLR₍₂₅₋₁₈₇₎ (SEQ ID NO: 7).

In some embodiments, the flexible linker comprises at least four contiguous amino acid residues selected from the group consisting of glycine, serine and/or alanine. Exemplary targeting moieties to be used in these embodiments (e.g. in the form of a protein consisting essentially of said anchoring component, said targeting component and said linker) include, but are not limited to, DARPin53F6 (SEQ ID NO: 11), CEACAM8₍₃₅₋₁₄₀₎ (SEQ ID NO: 12), and a selective ligand of the prostate-specific membrane antigen (PSMAL, e.g. the GTI peptide GTIQPYPFSWGY, SEQ ID NO: 37). In other embodiments, the linker is a flexible chemical linker (e.g. a poly(oxyethylene) linker or hydrocarbon linker). Exemplary targeting moieties to be used in these embodiments include, but are not limited to, a PSMAL comprising a Glu-NH—CO—NH-Lys pharmacophore, and folic acid.

In various embodiments the invention relates to a nucleic acid construct encoding the adapter molecule, a viral vector comprising the nucleic acid construct, wherein said construct is operably linked to one or more transcription regulation sequences, and a pharmaceutical composition comprising a therapeutically effective amount of the adapter molecule, which is specifically complexed in a non-covalent manner with particles of a virus or viral vector decorated with a vesiculovirus envelope glycoprotein (G) selected from the group consisting of: VSV-G, COV-G, and Maraba-G, to form adapter-modified viral particles, the pharmaceutical composition further comprising a pharmaceutically acceptable carrier, excipient or diluent. In another embodiment the invention relates to a process for producing the pharmaceutical composition, comprising contacting the particles of the virus or viral vector as disclosed herein with adapter molecules of the invention, so as to produce the adapter-modified viral particles.

In another embodiment, the adapter molecule is for use in delivering a virus or viral vector selectively into a target cell in a subject in need thereof, wherein the use comprises contacting particles of the virus or viral vector with said adapter molecule so as to produce adapter-modified viral particles, and administering the resulting adapter-modified viral particles to the subject. In another embodiment, adapter molecules and pharmaceutical compositions as disclosed herein are for use in treating a disease or condition in a subject in need thereof, e.g. a tumor or an inherited monogenic disorder.

These and other aspects and embodiments are described in greater detail below.

### Adapters

The invention in aspects and embodiments thereof relates to adapter molecules, also referred to herein as adapter proteins or adapters. An adapter molecule of the invention is a non-naturally occurring molecule or conjugate that comprises or consists of an anchoring component as disclosed herein covalently linked to a heterologous targeting component as disclosed herein.

In some embodiments, adapter molecules of the invention comprise an anchoring component covalently linked by a flexible linker to a targeting component, wherein:
a. the anchoring component comprises: at least one isolated CR motif selected from the group consisting of: (hLDLR CR2, hLDLR CR3, and homologs thereof,
b. the flexible linker comprises at least five contiguous amino acid residues selected from the group consisting of glycine, serine and/or alanine,
c. the targeting component comprises: a ligand of a receptor expressed preferentially on the surface of a mammalian target cell, or an antigen-binding molecule that selectively binds the receptor.

### Anchoring Component

As used herein, the term “anchoring component” refers to the virus-binding segment of the adapter. Anchoring components to be used in adapters of the invention contain at least one isolated Class A repeat (CR) motif, and are further characterized by structural and functional properties as disclosed herein.

In other embodiments, the anchoring component consists essentially of at least one isolated CR motif selected from the group consisting of: human low-density lipoprotein receptor (hLDLR) Class-A repeat 2 (hLDLR CR2) and hLDLR CR3, and optionally at least one of hLDLR CR1 and hLDLR CR4.

As disclosed herein, adapter molecules in accordance with the invention are capable of binding to an envelope glycoprotein of a vesiculovirus in a selective manner. According to embodiments of the invention, adapter molecules of the invention are capable of reversible binding to the envelope glycoprotein. Thus, adapter molecules in accordance with embodiments of the invention are stably complexed with the envelope glycoprotein under physiological conditions (e.g., at pH 7.35-7.45), and released from said envelope glycoprotein at endosomal pH (e.g., equal to or lower than pH 6.5, characteristic of the endosomal lumen).

In addition, adapter molecules of the invention facilitate endocytosis of a viral particle complexed with said adapter molecule selectively into said target cell. Without wishing to be bound by a specific theory or mechanism of action, adapter molecules of the invention provide for the production of adapter-viral particle complexes (also referred to herein as adapter-modified viral particles) characterized by remarkable and improved properties, including with respect to target selectivity, transduction efficacy, stability in vitro and in vivo, serum half-life, safety, therapeutic efficacy, and/or lower immunogenicity.

In one embodiment, the anchoring component comprises a C′-truncated ectodomain of a member of the LDLR family. In another embodiment, the member of the LDLR family is an LDLR polypeptide. In another embodiment, the member of the LDLR family is a human LDLR polypeptide. Thus, anchoring components in accordance with embodiments of the invention include non-naturally occurring fragments of LDLR, retaining the capacity to bind vesiculovirus envelope glycoproteins. According to exemplary embodiments, said anchoring component corresponds to positions 25-187, 25-149, 25-145, 66-105 or 107-144 of a human LDLR polypeptide (e.g., of human pro-LDLR set forth in accession no. NP_000518.1, SEQ ID NO: 15). Such C′ truncated LDLR ectodomain fragments, lacking other domains of the LDLR polypeptide such as the transmembrane domain, the beta-propeller domain and the EGFP domain, and retaining certain CR sequences as described above, are further referred to herein as isolated sLDLR polypeptides.

Accordingly, an isolated CR motif corresponds to a sequence of an art-recognized CR motif (such as human hLDLR CR motifs 1 to 3, including those set forth in SEQ ID NOs: 1-3 and their homologs described herein), and does not contain additional functional domains of LDLR family proteins (such as a β-propeller domain).

In various other embodiments, the anchoring component may include recombinant polypeptides, in which the CR motifs are arranged in an order that is distinct from the order in which they appear in the native LDLR polypeptides. For example, the CR motifs may be duplicated, deleted or rearranged, as long as the structural integrity of each CR sequence individually is maintained. In some exemplary embodiments, the anchoring component may include a CR2-CR2 polypeptide, a CR3-CR3 polypeptide, a CR2-CR3 polypeptide (comprising or consisting of an N′ CR2 motif and a C′ CR3 motif) or a CR3-CR2 polypeptide (comprising or consisting of an N′ CR3 motif and a C′ CR2 motif).

In some embodiments, the anchoring component comprises or consists of a plurality of the isolated CR motifs (e.g., 2-5, 2-3 or 3-4 isolated CR motifs as disclosed herein). In another embodiment, the anchoring component consists essentially of a plurality of CR2 sequences, CR3 sequences, or combinations thereof. In another embodiment, the anchoring component further comprises a CR1 sequence. In other embodiments, the anchoring component comprises, consists of, or consists essentially of a plurality of CR2 sequences of a human LDLR polypeptide (hLDLR CR2 sequences), hLDLR CR3 sequences, and combinations thereof. In another embodiment, the anchoring component further comprises a hLDLR CRI sequence. In other embodiments, the anchoring component consists essentially of a plurality of a hLDLR CRI sequence, a hLDLR CR2 sequences, and a hLDLR CR3 sequences.

Exemplary hLDLR CR1, CR2, and CR3 sequences are as set forth in SEQ ID NOs: 1-3 below, respectively, in which conserved residues are underlined and residues involved in direct contact with VSV-G residues are in bold and underlined:

(hLDLR CR1, SEQ ID NO: 1)
DRCERNEFQCQDGKCISYKWVCDGSAECQDGSDESQETCL;
(hLDLR CR2, SEQ ID NO: 2)
VTCKSGDFSCGGRVNRCIPQFWRCDGQVDCDNGSDEQGCP;
(hLDLR CR3, SEQ ID NO: 3)
KICSQDEFRCHDGKCISROFVCDSDRDCLDGSDEASCP.

In another embodiment, CR2 and/or CR3 sequences homologous to hLDLR CR2 and/or hLDLR CR3, derived from other members of the LDLR family, may be used. As disclosed herein, such homologous sequences typically comprise conserved residues as indicated above and a high degree of homology to hLDLR CR2 and/or hLDLR CR3, so as to allow high-affinity binding to a vesiculo-G protein (with Kd of less than 1 micromolar). Thus, CR2 and CR3 sequences to be used in embodiments of the invention typically comprise six conserved cysteine residues, and four conserved aspartic and/or glutamic acid residues (involved in Ca²⁺ binding), at positions homologous to those indicated above with respect to hLDLR CR2 and hLDLR CR3 sequences. Further, such homologous sequences advantageously contain FWRCDXQ and/or QFVCDXD motifs (X representing a small amino acid residue such as G or S).

More specifically, CR2 and CR3 homologs are typically about 40 aa in length (e.g., 36-42, or 35-45). These motifs further typically contain three disulfide bridges within each motif, formed by their six conserved cysteine residues. A calcium ion binding pocket, consisting of Asp and Glu amino acid residues, is also present in CR2 and CR3 homologs to be used in accordance with the invention. In addition to the above-mentioned conserved structural elements, such homologs also share a high degree of homology to hLDLR CR2 and/or hLDLR CR3, e.g., at least 60% and typically at least 70%, 80%, 90%, 93%, 95%, 97% or 99%. Each possibility represents a separate embodiment of the invention.

For example, the homolog CR motif may be derived from a member of the LDLR family selected from the group consisting of low-density lipoprotein receptor (LDLR), very-low-density-lipoprotein receptor (VLDLR), Low-density lipoprotein receptor-related protein 8 (LRP8, ApoER2), Low density lipoprotein receptor-related protein 4 (LRP4), LDLR-related protein 1 (LRP1), LDLR-related protein 1b (LRP1b), and Megalin, and retains the ability to be complexed with the vesiculovirus envelope glycoprotein under physiological conditions. In another embodiment, said member of the LDLR family is selected from the group consisting of human LDLR. LRP1, LRP8 and VLDLR receptors and said composition comprises an effective amount of said PCSK9 polypeptide. Each possibility represents a separate embodiment of the invention. In another embodiment, said member of the LDLR family is selected from the group consisting of human LDLR. LRP1, LRP8 and VLDLR receptors, which are particularly advantageous for use concomitantly with administration of a PCSK9 polypeptide, as described in further detail below.

In this context, a CR motif “derived from” a particular receptor indicates that said motif may be obtained or isolated from said receptor. CR motifs of various LDLR family members are known in the art, for example the location of CR repeats of LDLR family proteins are explicitly identified in their respective GenBank entries as set forth in below and incorporated herein by reference: low-density lipoprotein receptor (LDLR, NP_000518.1); very low-density lipoprotein receptor (VLDLR, NP_003374.3); low-density lipoprotein receptor-related protein 8 (LRP8, NP_004622.2); low-density lipoprotein receptor-related protein 4 (LRP4, NP_002325.2); low-density lipoprotein receptor-related protein 1 (LRP1, NP_002323.2); low-density lipoprotein receptor-related protein 2 (Megalin, NP_004516); low-density lipoprotein receptor-related protein 1B (LRP1B, NP_061027); low-density lipoprotein receptor-related protein 6 (LRP6, EAW96253.1); low-density lipoprotein receptor-related protein 5 (LRP5, NP_002326.2); low-density lipoprotein receptor class A domain-containing protein 3 (NP_777562.1); low-density lipoprotein receptor class A domain-containing protein 4 (NP_001365029.1); low-density lipoprotein receptor-related protein 12 (LRP12, NP_038465.1); low-density lipoprotein receptor-related protein 10 (LRP10, NP_054764.2); low-density lipoprotein receptor-related protein 3 (LRP3, NP_002324.2); low-density lipoprotein receptor-related protein 105 (LRP105, BAA32330.1); and low-density lipoprotein receptor-related protein 11 (LRP11, KAI4020222.1).

### Targeting Component

As used herein, the term “targeting component” refers to the segment of the adapter that can bind the target cell intended to be treated or modulated. Targeting components to be used in adapters of the invention contain a target-specific antigen-binding molecule or ligand as disclosed herein, and are further characterized by structural and functional properties as further detailed and exemplified herein.

The term “preferentially expressed” with respect to a receptor being preferentially expressed on a certain cell or tissue, means that the receptor is expressed at a significantly higher level in that cell or tissue than in other cells and tissues of the subject. For example, TAA include surface receptors preferentially expressed on tumor cells. Particular examples of preferentially expressed receptors are the tissue-specific and tumor-specific molecules listed in Table 1, along with the target cells on which they are expressed preferentially, and exemplary ligands and/or antigen binding moieties (collectively referred to in Table 1 as “exemplary targetors”).

TABLE 1
Exemplary target molecules

| | Full name & | | | UniProt | Exemplary |
|---|---|---|---|---|---|
| Molecule | synonyms | Description/ Use | Target cells | no. | targetors |
| CD8 | Cluster of | Transmembrane | Cytotoxic T cells, | P01732 | DARPin53F6 |
| | differentiation 8 | glycoprotein that | NK cells, certain | | |
| | | serves as a co-receptor | other immune cells | | |
| | | for the T-cell receptor | | | |
| | | (TCR) | | | |
| | | Immunotherapy | | | |
| | | (generation of tumor- | | | |
| | | specific immune cells) | | | |
| CD56 | Cluster of | Homophilic surface | NK cells, neurons, | P13591 | CD56 (CD56 |
| | differentiation | glycoprotein having a | glia, skeletal | | binding |
| | 56 | role in cell-cell | muscle, certain | | domain) / |
| | NCAM | adhesion, neurite | other immune cells | | CD56-specific |
| | (Neutral Cell | outgrowth, synaptic | | | antibody/ |
| | Adhesion | plasticity, and learning | | | DARPin |
| | Molecule, or | and memory | | | |
| | NCAM1) | Immunotherapy | | | |
| | | (generation of tumor- | | | |
| | | specific immune cells) | | | |
| PSMA | Prostate- | Membrane zinc | Prostate secretory- | Q04609 | PSMAL (e.g., |
| | specific | metalloenzyme that | acinar epithelium, | | Glu-NH-CO- |
| | membrane | catalyzes the | enhanced in | | NH-Lys |
| | antigen | hydrolysis of N- | prostate tumor | | pharmacophore |
| | Glutamate | acetylaspartylglutamate | cells. | | ligands and GTI |
| | carboxypeptidase | (NAAG) to | | | peptide ligands) |
| | II (GCPII), N- | glutamate and N- | | | |
| | acetyl-L- | acetylaspartate | | | |
| | aspartyl-L- | (NAA). | | | |
| | glutamate | Cancer therapy (TAA) | | | |
| | peptidase I | | | | |
| | (NAALADase | | | | |
| | I), NAAG | | | | |
| | peptidase, folate | | | | |
| | hydrolase 1. | | | | |
| CEACAM6 | Carcinoembryonic | Glycosyl phosphatidyl | alveolar and | P40199 | CEACAM8 |
| | antigen- | inositol (GPI) | airway epithelial | | (CEACAM6- |
| | related cell | anchored cell surface | cells, tumor cells | | binding |
| | adhesion | glycoprotein. Plays a | | | domain) |
| | molecule 6 | role in cell adhesion | | | |
| | CD66c, CEAL, | and tumor progression | | | |
| | NCA | Lung-specific gene | | | |
| | | therapy, cancer | | | |
| | | therapy (TAA) | | | |
| CEACAM1 | Carcinoembryonic | Mediates cell-cell | leukocytes, | P13688 | CEACAM8, |
| | Antigen- | adhesion. Roles in the | epithelia, and | | CEACAM5 |
| | related Cell | differentiation and | endothelia, tumor | | (CEACAM1- |
| | Adhesion | arrangement of tissue | cells | | binding |
| | Molecule 1 | three-dimensional | | | domain) |
| | CD66a, BGP, | structure, | | | |
| | BGP1 | angiogenesis, | | | |
| | | apoptosis, tumor | | | |
| | | suppression, | | | |
| | | metastasis, and the | | | |
| | | modulation of innate | | | |
| | | and adaptive immune | | | |
| | | responses | | | |
| | | Lung-specific gene | | | |
| | | therapy, cancer | | | |
| | | therapy (TAA) | | | |
| c-KIT | Proto-oncogene | Tyrosine kinase | Expressed in | P10721 | SCF (c-KIT- |
| | c-KIT | receptor. Roles in | hematopoietic stem | | binding |
| | CD117, PBT, | proliferation, | cells and tumor | | domain) |
| | SCFR, KIT | differentiation, and | cells | | |
| | proto-oncogene | recruitment of | | | |
| | receptor | progenitor cells | | | |
| | tyrosine kinase | Cancer therapy (TAA) | | | |
| Folate | FOLR1 | HSC-specific gene | Hematopoietic | P15328 | Folic acid |
| Receptor | Folate receptor | therapy, cancer | stem cells (HSC) | | |
| | alpha, Adult | therapy (TAA) | and tumor cells | | |
| | folate-binding | | | | |
| | protein (FBP), | | | | |
| | Folate receptor | | | | |
| | 1, Folate | | | | |
| | receptor, adult, | | | | |
| | KB cells FBP, | | | | |
| | Ovarian tumor- | | | | |
| | associated | | | | |
| | antigen MOv18 | | | | |

As used herein, the term “antigen binding molecule” refers in its broadest sense to a molecule that specifically binds an antigenic determinant. Examples of antigen binding molecules are antibodies, including antigen-binding antibody fragments, and scaffold antigen binding proteins such as DARPin molecules.

In another embodiment, the antigen-binding molecule is a Designed Ankyrin Repeat Protein (DARPin), an antibody, or an antigen-binding fragment thereof. In another embodiment, said surface-expressed receptor is selected from the group consisting of: EGF-receptor (EGFR), ErbB2, HER2, CEA, CEACAM family receptors (e.g., CEACAM1 and CEACAM6), emmprin, endoglin, EpCAM, folate receptor (FOLR1), GRP78, IGF-1R, mesothelin, Muc-1, PSCA, prostate-specific membrane antigen (PSMA), CD8, and CD56. Other exemplary surface-expressed receptors are e.g., αvβ3 integrin, bombesin receptor, uPAR, Tn antigen, MT6-MMP, MT1-MMP, cMET/HGFR, matriptase, FAP-α, EphA2, CXCR4, CD44 v6, and CD13. In another embodiment, said surface-expressed receptor is selected from the group consisting of: human CEACAM6, human CEACAM1, human c-KIT, human PSMA, and human CD8. In another embodiment, said surface-expressed receptor is selected from the group consisting of: human CEACAM6, human CEACAM1, human c-KIT, human PSMA, human CD8 and human FOLR1. In another embodiment, said surface-expressed receptor is selected from the group consisting of: human CEACAM6, human CEACAM1, human PSMA, human CD8 and human FOLR1. In another embodiment, said surface-expressed receptor is selected from the group consisting of: human CEACAM6, human CEACAM1, and human CD8. Each possibility represents a separate embodiment of the invention.

The terms “antibody” or “antibodies” as used herein refer to an antibody, preferably a monoclonal antibody, or fragments thereof, including, but not limited to, a full-length antibody having a human immunoglobulin constant region, a monoclonal IgG, a single chain antibody, a humanized monoclonal antibody, an F(ab′)₂ fragment, an F (ab) fragment, an Fv fragment, a labeled antibody, an immobilized antibody and an antibody conjugated with a heterologous compound. Each possibility represents a separate embodiment of the invention. In one embodiment, the antibody is a monoclonal antibody (mAb). In another embodiment, the antibody is a humanized antibody.

Methods of generating monoclonal and polyclonal antibodies are well known in the art. Antibodies may be generated via any one of several known methods, which may employ induction of in vivo production of antibody molecules, screening of immunoglobulin libraries, or generation of monoclonal antibody molecules by continuous cell lines in culture. These include, but are not limited to, the hybridoma technique, the human B-cell hybridoma technique, and the Epstein-Barr virus (EBV)-hybridoma technique. Besides the conventional method of raising antibodies in vivo, antibodies can be generated in vitro using phage display technology, by methods well known in the art (e.g., Current Protocols in Immunology, Colligan et al (Eds.), John Wiley & Sons, Inc. (1992-2000), Chapter 17, Section 17.1).

The term “designed ankyrin repeat protein” or “DARPin” refers to a non-naturally-occurring (engineered) antibody mimetic in which the antigen-binding domain comprises or consists of ankyrin repeat motifs. Naturally occurring ankyrin repeats are conserved motifs of about 33 residues found in ankyrin proteins, a protein class that is mediating high-affinity protein-protein interactions in nature. DARPins typically contain a plurality (e.g., 2-5, 2-3 or 3-4) of ankyrin repeat motifs, engineered to contain one or more amino acid mutations (e.g., substitutions) affecting for example their binding affinity to a target molecule, their cell surface expression, and the like. The plurality of ankyrin repeat motifs within a DARPin are typically flanked by conserved N- and C-capping repeats defining the ankyrin framework and providing further structural stability. The target interaction residues are mainly found in the β-hairpin and the exposed part of the first α-helix within the characteristic fold of the ankyrin repeat motifs, and endow the DARPin molecule with high specificity and high binding affinity. The term as used herein further encompasses LoopDARPin, which are modified DARPin molecules in which the LoopDARPin scaffold replaces the concave binding surface of the DARPin by one with a protrusion (loop) in the middle. DARPins that bind to specific targets can be identified by screening combinatorial libraries of DARPins and selecting those with desired binding properties for the target. Such screening methods are described in, e.g., Muench et al., Molecular Therapy, 16(4), 686-693, 2011. For example, ribosomal display or phage display methods can be used to select target-specific DARPins from diverse libraries.

In yet other embodiments, the targeting component is a ligand of said receptor, e.g., a naturally-occurring ligand known in the art as a binding partner of said receptor, or a fragment thereof comprising at least the receptor binding domain. Natural ligands of receptors are known to specifically bind and cause a change in the receptor so as to effect a change in its activity or a response in cell that expresses that receptor. As disclosed herein, ligands to be used in the context of the invention are advantageously selective ligands, which bind specifically to the receptor in question and do not substantially bind to other receptors expressed on non-related cells at the site of administration. Such exemplary ligands are listed below.

The terms “binding domain”, “binding site”, or “binding portion” as used herein, refer to the portion, region, or site of a molecule, in particular a polypeptide, that retains the structural properties that mediate specific binding with a target molecule such as an antigen, ligand or receptor. Receptor binding domains of ligands disclosed herein have been identified and readily recognized by the skilled artisan. Exemplary receptor binding domains of ligands that may be used in advantageous embodiments of the invention are further listed below.

For example, CEA Cell Adhesion Molecule 8 (CEACAM8, also known as CD66b, CD67, CGM6, and NCA-95) is a surface protein involved in cell adhesion, migration, and pathogen binding. CEACAM8 (including receptor-binding fragments thereof) may be used as a targeting component in adapter molecules of the invention targeting lung epithelia and tumor cells that express CEACAM6 and/or CEACAM1. CEACAM8 specifically binds to CEACAM6 and CEACAM1 via its receptor-binding domain (amino acids 35-140 in human CEACAM8, accession no. NP_001807.2, SEQ ID NO: 38). Human CEACAM8 is further described in UniProt no. P31997.

Carcinoembryonic antigen-related cell adhesion molecule 5 (CEACAM5, also known as CD66c and CEA), is another surface protein involved in cell-cell adhesion. CEACAM5 (including receptor-binding fragments thereof) may be used as a targeting component in adapter molecules of the invention, targeting lung epithelial cells and certain tumor cells that express CEACAM1. CEACAM5 specifically binds to CEACAM1 via its receptor-binding domain (amino acids 35-144 in human CEACAM5, SEQ ID NO: 39). Human CEACAM5 is further described in UniProt no. P06731.

Stem cell factor (SCF, also known as KIT ligand, KITL, FPH2, FPHH, KL-1, and MGF) is known to exist in two forms: a transmembrane ligand of the c-KIT receptor and a soluble cytokine. SCF (including receptor-binding fragments thereof) may be used as a targeting component in adapter molecules of the invention targeting hematopoietic stem cells and tumor cells expressing the c-KIT receptor (CD117). SCF specifically binds to c-KIT via its receptor-binding domain (soluble SCF, corresponding to amino acids 26-190 in human SCF, NM_003994.6). Human SCF is further described in UniProt no. P21583, and murine SCF is further described in UniProt no. P20826.

In other embodiments, the ligand is a synthetic ligand or a chemically-modified ligand. For example, synthetic ligands that selectively bind to PSMA but not to folate receptor (FR) family members have been described (Eder et al., 2012. Bioconjugate Chemistry, 23(4): p. 688-697; Wei Jin et al., Int J Pharm. 2016 Nov. 20; 513(1-2): 138-147, incorporated herein by reference). For instance, a selective ligand of human PSMA (PSMAL) has been identified as having the Glu-NH—CO—NH-Lys pharmacophore (Eder et al., 2012. ibid). In the context of the present invention, this PSMAL may conveniently be conjugated to SH groups in cysteine residues, forming a disulfide bond, typically via a flexible linker such as a 2-10 hydrocarbon linker. For example, without limitation, said PSMAL may be used in the form of (7S,14S,18S)-7-amino-1-(3-(2,5-dioxo-2,5-dihydro-1H-pyrrol-1-yl)phenyl)-1,8,16-trioxo-2,9,15,17-tetraazaicosane-14,18,20-tricarboxylic acid, comprising said pharmacophore and said flexible linker. Another selective ligand of human PSMA used in embodiments of the invention is a GTI peptide (GTIQPYPFSWGY, SEQ ID NO: 37). This peptide was described in Wei Jin et al., 2016 ibid as having high-affinity for the PSMA ectodomain and PSMA-positive prostate cancer cells. These ligands may be used as a targeting component in adapter molecules of the invention targeting prostate tumor cells expressing PSMA.

Folic acid (FA. [(2S)-2-[[4-[(2-amino-4-oxo-1H-pteridine-6-yl)methylamino]benzoyl]amino] pentanedioic acid]) is the free acid form of folate, also known as vitamin B9 and folacin, which is one of the B vitamins. FA is specifically bound by folate receptor FOLR1, expressed on the surface of dividing cells (such as mobilized HSC and tumor cells), while folate and other derivatives are also bound by other folate receptors. In the context of the present invention, FA may conveniently be conjugated to α-amines and/or ε-amines of polypeptide chains by FA-N-hydroxysuccinimide ester (FA-OSu, advantageously via a flexible linker such as poly(oxyethylene) (also referred to herein as polyethylene glycol or PEG linkers). For example, FA may be coupled to cysteine to form FA-Cys, which can be reacted with maleimide-PEG-COOSu to form FA-Cys-Mal-PEG-COOSu, and then conjugated to α-amines and/or ε-amines of polypeptide chains by protocols as disclosed and exemplified herein.

In another embodiment, the ligand is selected from the group consisting of: human CEACAM8, human SCF, a human PSMA ligand (PSMAL), and fragments thereof comprising at least the receptor binding domain. In another embodiment, the targeting component is an antibody directed to human CEACAM6, human CEACAM1, human c-KIT or human PSMA, or comprises an antigen-binding fragment thereof. In another embodiment, the targeting component is a DARPin directed to human PSMA, human CD8 or human CD56. In yet another embodiment, the ligand is a natural ligand of a receptor as disclosed herein, or comprises the receptor binding domain thereof. Each possibility represents a separate embodiment of the invention.

The term “directed” as used herein refers to specific binding under physiological conditions. For example, an antigen-binding protein such as antibody or a DARPin molecule directed to an antigen is capable of specifically binding said antigen under physiological conditions.

In a particular embodiment, said targeting component is (or comprises) DARPin53F6 (SEQ ID NO: 11). In another particular embodiment said targeting component is (or comprises) CEACAM8₍₃₅₋₁₄₀₎ (SEQ ID NO: 12). In another particular embodiment said targeting component is (or comprises) murine pro-KIT-ligand₍₂₆₋₁₉₀₎ (mSCF, SEQ ID NO: 13). In another particular embodiment said targeting component is (or comprises) human pro-KIT-ligand₍₂₆₉₋₇₆₃₎ (hSCF, SEQ ID NO: 14). In another particular embodiment said targeting component is (or comprises) a GTI peptide (GTIQPYPFSWGY, SEQ ID NO: 37). In another particular embodiment said targeting component is (or comprises) a Glu-NH—CO—NH-Lys pharmacophore. In another particular embodiment said targeting component is (or comprises) folic acid.

### Linkers and Conjugation

In this disclosure, the terms “covalently linked” or “covalently bound” or “linked by a covalent bond” and similar expressions are interchangeable, and mean that atoms, molecules, or moieties of a molecule are linked together by one or more covalent bonds (e.g., peptide bonds, disulfide bonds or amide bonds). In various embodiments, the moieties or molecules may be conjugated by suitable means including chemical conjugation, recombinant techniques or enzymatic activity, either directly or via one or more additional moieties that serve as linking agents (linkers). In some embodiments (for example when the linker is part of a fusion protein), the linker is comprised of one or more amino acids (peptide linker). In other embodiments, said linker is a chemical linker, which may comprise a hydrocarbon chain and/or additional functional groups providing for chemical conjugation.

In another embodiment, the adapter molecule is a fusion protein. The term “fusion protein” refers to a chimeric polypeptide formed by the joining of two or more heterologous peptides, polypeptides or protein domains through a peptide bond formed between the amino terminus of one polypeptide and the carboxyl terminus of another polypeptide. The two or more moieties within a fusion protein may be linked directly or via a peptide linker. Fusion proteins are conveniently prepared using conventional techniques in molecular biology to join the two genes in frame into a single nucleic acid, and then expressing the nucleic acid in an appropriate host cell under conditions in which the fusion protein is produced.

In another embodiment, the linker comprises or consists of at least four, or in other embodiments, at least five contiguous glycine, serine and/or alanine residues. In another embodiment, said linker is a flexible linker. A flexible linker (e.g., for use in fusion proteins of the invention) is a peptide segment that typically comprises a plurality of small amino acid residues lacking charge, high polarity and free SH groups. Thus, while some linkers may be synthesized with cysteine residue in order to chemically conjugate non-polypeptide linkers, such cysteines no longer contain free SH groups following the conjugation. Flexible linkers are further characterized by lack of strong hydrophobic or polar interactions. In another embodiment said linker is a flexible linker comprising at least four contiguous glycine, serine and/or alanine residues. Linkers to be used in adapter molecules of the invention are typically and conveniently less than 20 amino acids (aa) in length, more typically 4-15, 4-8, 4-5, 4-6, 5-10 or 4-12 aa in length.

In other embodiments, for example when one or more component of an adapter molecules is a non-peptide chemical moiety, the linker may be a non-peptide linker (also referred to herein as a chemical linker). Flexible chemical linkers for use in adapters of the invention typically comprises a poly(oxyethylene) (PEG) chain having from 4 to 24 (e.g., 4-8, 8-12, 5-20, 7-9 or 8-24 oxyethylene units), or a 2-10 carbon chain (e.g., 4-8, 2-5, 5-10 or 5-7 alkyl chains), providing structural flexibility. It is to be understood that ranges of PEG units as provided herein refer to an individual length of a PEG linker in a specific adapter molecule, e.g., “a poly(oxyethylene) having 4 to 24 units” refers to each linker within this range, wherein each possibility represents a separate embodiment of the invention. The term “poly(oxyethylene), poly(ethylene glycol) and PEG) are used herein interchangeable to define a linker that comprises the moiety: (OCH₂CH₂)ₙ.

In addition, chemical linkers for use in adapters of the invention may contain functional groups for conjugating the chemical moiety to the peptide moiety, for example a triazole moiety, a S-succinimide bond linking the chemical moiety to a cysteine residue, or an amino group to which the non-peptide ligand is attached at one end and an activated ester such as N-hydroxysuccinimide ester at the other end, which enables linking the chemical moiety to amino residues. Such chemical linkers may conveniently be conjugated to the polypeptide component of the adapter molecule via cysteine or lysine residues, e.g., by the methods disclosed and exemplified herein.

For example, A linker precursor comprising a maleimide group may react with the surface-exposed cysteine at position 148 of sLDLR₍₂₅₋₁₄₉₎ to form a sulfur-succinimide bond at position 3 of the maleimide connecting the linker to the protein. Alternatively, one of the linker precursor and the protein may be functionalized with an azide group and the other with an alkyne group, such that a click chemistry-type reaction there-between will result in a triazole link in the conjugate.

In another example, PEGn compounds having PEG spacers with maleimide (Mal) at one end and and COOSu at the other end are available from BroadPharm. These are unbranched, hydrophilic, discrete-length molecules having the form of Mal-PEGn-COOSu, where the subscript ‘n’ denotes 4, 8, 12, or 24 oxyethylene units. It can be appreciated by the person having ordinary skill in the art that lengths other than 4, 8, 12 and 24 (e.g., 10 or 18) may also apply for the purpose of the present linker. The Mal of each compound provides specific targets for conjugating SH-bearing ligands to form a ligand-PEGn-COOSu moiety. For example, a ligand linked to a flexible PEG linker comprising a PEG chain of at least 2 monomeric oxyethylene units, preferably consisting of 8 to 24 units (degree of polymerization=8 to 24, e.g., FA-Cys-Mal-PEGs-COOSu), may be used to conjugate FA ligand to amine residues of lysine side chains and/or to the amine termini of the polypeptide chain.

### Exemplary Molecules and Properties

In another embodiment, said adapter molecule (e.g., fusion protein) is 250-350 amino acids in length. In another embodiment, said adapter molecule is about 300 amino acids in length.

In another embodiment, said adapter molecule consists essentially of said anchoring component, said targeting component and said linker. In another embodiment, said adapter molecule further comprises a protein tag such as a histidine tag. It is to be understood, that adapters of the invention as disclosed herein may be used with or without their indicated histidine tags. For example, adapters intended for in vivo administration to a subject (for example for the generation of CAR T cells in vivo) are advantageously devoid of their respective histidine tags. By means of a non-limiting example, sLDLR-DARPin53F6 (SEQ ID NO: 16, described in Example 14 below), optionally excludes its histidine tag at positions 286-294.

In another embodiment, the anchoring component can be complexed with a vesiculovirus envelope glycoprotein under physiological conditions. In another embodiment, the vesiculovirus is selected from the group consisting of vesicular stomatitis virus (VSV), Cocal virus (COV), and Maraba virus (rhabdovirus). In another embodiment, said adapter molecule binds reversibly to VSV envelope glycoprotein (VSV-G). In another embodiment, said adapter molecule binds to VSV-G selectively.

In another embodiment, the anchoring component consists essentially of hLDLR CR1, CR2 and CR3, and the targeting component selectively binds to human CEACAM6 (hCEACAM6). In another embodiment, the anchoring component consists essentially of hLDLR CR1, CR2 and CR3, and the targeting component comprises a receptor-binding portion of human CEACAM8 (hCEACAM8). In another embodiment, the anchoring component consists essentially of hLDLR CR1, CR2 and CR3, and the targeting component comprises an antigen-binding portion of an antibody directed to hCEACAM6. In another embodiment, the anchoring component consists essentially of hLDLR CR1, CR2 and CR3, and the targeting component selectively binds to human SCF receptor c-KIT. In another embodiment, the anchoring component consists essentially of hLDLR CR1, CR2 and CR3, and the targeting component comprises a receptor-binding fragment of human c-KIT ligand (SCF). In another embodiment, the anchoring component consists essentially of hLDLR CR1. CR2 and CR3, and the targeting component comprises an antigen-binding portion of an antibody directed to human c-KIT. In another embodiment, the anchoring component consists essentially of hLDLR CR1, CR2 and CR3, and the targeting component comprises an antigen-binding portion of an antibody directed to human CD8, or a DARPin selected for very high affinity towards human CD8. In another embodiment, the anchoring component consists essentially of hLDLR CR1, CR2 and CR3, and the targeting component comprises an antigen-binding portion of an antibody directed to human CD56, or a DARPin selected for very high affinity towards human CD56. In another embodiment, the anchoring component consists essentially of hLDLR CR1, CR2 and CR3, and the targeting component selectively binds to the human PSMA. In another embodiment, the anchoring component consists essentially of hLDLR CR1, CR2 and CR3, and the targeting component comprises a selective human PSMA ligand (PSMAL). In another embodiment, the anchoring component consists essentially of hLDLR CR1, CR2 and CR3, and the targeting component comprises an antigen-binding portion of an antibody directed to human PSMA, or a DARPin selected for very high affinity towards human PSMA.

In another embodiment, said adapter molecule has an amino acid sequence selected from the following, optionally excluding their respective C-terminal histidine tags, as follows:
sLDLR-DARPin53F6 (SEQ ID NO: 16), optionally excluding the histidine (His) tag at positions 286-294,
sLDLR-CEACAM8 (SEQ ID NO: 17), optionally excluding the His tag at positions 296-304,
sLDLR-SCF (SEQ ID NO: 18), optionally excluding the His tag at positions 295-304,
sLDLR-hSCF (SEQ ID NO: 19), optionally excluding the His tag at positions 295-304, and
sLDLR-PSMAL, consisting of sLDLR₂₅₋₁₄₉ (SEQ ID NO: 6) chemically conjugated to a PSMAL as disclosed herein, for example to sLDLR₍₂₅₋₁₄₉₀ chemically conjugated to (7S,14S,18S)-7-amino-1-(3-(2,5-dioxo-2,5-dihydro-1H-pyrrol-1-yl)phenyl)-1,8,16-trioxo-2,9,15,17-tetraazaicosane-14,18,20-tricarboxylic acid (which comprises a Glu-NH—CO—NH-Lys pharmacophore and a flexible carbohydrate linker).

Additional exemplary adapters disclosed herein include:
GTI-sLDLR₍₂₅₋₁₄₅₎ (SEQ ID NO: 40), optionally excluding the C-terminal His tag; and
FA-PEG-sLDLR, corresponding to sLDLR₍₂₅₋₁₄₅₎ (SEQ ID NO: 5) chemically conjugated to folic acid via a flexible poly(oxyethylene) linker (e.g. an amino-PEGs-COOH linker, resulting in the FA-PEG8-sLDLR adapter).

These and other adapter molecules as will be described in greater detail below are collectively referred to herein as the adapter molecules of the invention. Each possibility represents a separate embodiment of the invention.

Typically and advantageously, adapter molecules of the invention include the anchoring component, the targeting component and the linker as disclosed herein, and substantially lack additional functional domains or motifs. In particular, advantageous adapter molecules in accordance of the invention lack additional non-related motifs derived from LDLR family polypeptides. For example, adapter molecules of the invention were unexpectedly determined to mediate highly selective and potent viral therapy using short anchoring sequences derived from C′-truncated human LDLR, which notably lack the LDLR beta-propeller domain (amino acid residues 396-664 of human pro-LDLR), hitherto considered to mediate ligand dissociation via intramolecular competition at low pH, thereby facilitating endosomal release of the ligand. In other words, while the beta-propeller domain was considered to mediate reversible binding of LDLR to LDLR-targeting viruses, thereby facilitating endosomal release and effective infection, and preventing lysosomal degradation of said viruses, the present invention demonstrates highly effective infection by viral particles complexed with adapters of the invention, in the absence of the beta propeller region and its characteristic YWTD repeat motifs.

### Constructs, Vectors and Adapter Modified Viral Particles

In another embodiment there is provided a nucleic acid construct encoding an adapter molecule of the invention. In another embodiment, the nucleic acid construct comprises a nucleic acid sequence as set forth hereinbelow. The term “construct” as used herein refers to a polynucleotide comprising a nucleic acid sequence of interest (e.g., encoding an adapter according to the present invention). In some embodiments, the construct is an expression construct, in which the nucleic acid sequence is operably linked to a promoter and optionally other transcription regulation sequences.

The phrase “operably linked” refers to a nucleic acid sequence linked a to a transcription control sequence in a manner such that the molecule is able to be expressed when transfected (i.e., transformed, transduced, infected, or transfected) into a host cell. Transcription regulation or transcription control sequences are sequences, which control the initiation, elongation, and termination of transcription. Particularly important transcription control sequences are those which control transcription initiation, such as promoter, enhancer, operator and repressor sequences. A variety of such transcription control sequences are known to those skilled in the art. Exemplary suitable transcription control sequences include those that function in animal, bacteria, helminth, yeast and insect cells. In some embodiments, constructs of the invention comprise mammalian transcription control sequences, e.g., human regulatory sequences, and, optionally and additionally, other regulatory sequences.

In another embodiment the invention provides a viral vector comprising the nucleic acid construct, wherein said construct is operably linked to one or more transcription regulation sequences. In another embodiment, said viral vector is selected from the group consisting of a recombinant vesicular stomatitis virus (VSV), Cocal virus (COV), and Maraba virus (Maraba) vectors.

In another embodiment, the vector further comprises a nucleic acid sequence encoding a vesiculovirus envelope glycoprotein. In various embodiments, the vesiculovirus envelope glycoprotein is selected from the group consisting of: VSV-G, COV envelope glycoprotein (COV-G) and Maraba virus envelope glycoprotein (Maraba-G), collectively referred to herein as “vesiculo-G proteins”. In another embodiment, said viral vector is selected from the group consisting of a VSV vector, a COV vector and a Maraba virus vector. Each possibility represents a separate embodiment of the invention.

In another aspect, there is provided a viral particle complexed with an adapter protein of the invention. As disclosed herein, viral particles decorated with vesiculovirus envelope glycoproteins (for example viruses expressing vesiculo-G proteins naturally, or vesiculo-G protein-pseudotyped LVV) can be complexed with adapter molecules of the invention in a selective non-covalent (and typically reversible) manner. As is further disclosed herein, such complexed viral particles, also referred to herein as adapter-modified viral particles, are capable of being endocytosed selectively into a mammalian target cell characterized by surface expression of the receptor to which the cell-targeting component is directed. Without wishing to be bound by a specific theory or mechanism of action, adapter-modified viral particles of the invention manifest clinically-relevant stability (characteristic of VSV or VSV-G-pseudotyped LVVs) under physiological conditions in vivo, with highly improved target selectivity, and while maintaining high transduction capacity, thus exhibiting marked improvement over hitherto suggested vectors and adapters.

The term “reversible” or “binds reversibly” refers to the binding affinity of two molecules or entities, in particular to their ability to form a non-covalent complex that is stable under certain conditions but can be disrupted, resulting in the separation (dissociation) of the resulting complex, under certain other conditions. In the context of the present invention, the term relates in particular to the ability of the molecules or entities (for example a receptor and a ligand, or an adapter protein and a viral envelope protein) to form a specific binding complex that is stable under physiological conditions (characterized for example by a pH of 7.35-7.45), and is disrupted and substantially dissociated in conditions characteristic of the endosomal lumen (for example at a pH of 6-6.5). in contradistinction, certain high-affinity antigen-binding molecules such as antibodies and DARPin molecules may bind their antigen with sufficient affinity such that the resulting binding complex may be substantially stable under both conditions.

In another embodiment, the adapter molecule of the invention is specifically complexed in a non-covalent manner with particles of a virus or viral vector decorated with a vesiculovirus envelope glycoprotein (G) selected from the group consisting of: VSV-G, COV-G Maraba-G, to form adapter-modified viral particles. The term “VSV-G”, as used herein, relates to the spike glycoprotein G of the vesicular stomatitis virus. The term includes both wild-type VSV-G and VSV-G variants known in the art (that retain the membrane-fusing properties of the wild-type protein). In some embodiments, complement-resistant versions of VSV-G, e.g., VSV-G bearing the T230N and T368A mutations (Hwang et al., Gene Therapy, 2013. 20(8): p. 807-815) that may diminish the risk of a priori vector neutralization, may be used in embodiments of the invention.

The term “non-covalently linked” or “complexed in a non-covalent manner” comprises a non-covalent interaction that differs from a covalent bond in that it does not involve the sharing of electrons, but rather involves more dispersed variations of electromagnetic interactions between molecules or within a molecule, such as such as ion bonds, hydrogen bonds, Van der Waals force and hydrophobic interactions. The term “specifically complexed” refers in particular to such complexes that remain physically associated under the conditions in which the complex is used. For example, adapter-modified viral particles in accordance with the invention remain stably complexed under physiological conditions, as disclosed and exemplified herein.

As used herein the term “physiological conditions” refers to aqueous conditions that are normally present in a living organism, including in particular in an extracellular space or on an extracellular surface (e.g., on a cell membrane). In the context of the invention, the term encompasses the ranges of biochemical parameters such as temperature, pH and ionic strength, that are present when cell surface molecules are active or express their activities (e.g., enzymatic activity, ligand binding and the like). In particular, physiological conditions are characterized by a temperature of 36-38° C., a pH of 7.35-7.45 and an ionic strength range of about 110 mM to about 260 mM.

The term “particle” when used herein in the context of viruses and viral vectors, refers to a viral particle, which includes a genetic material (such as a DNA or RNA viral genome or vector), that is associated with and typically packaged within a coat of a viral protein or proteins, and, in some cases, a lipidic envelope. For the purpose of the present invention, the term encompasses both infective virions and replication-attenuated virions and virus-like particles.

As used herein, the term “decorated” with respect to a viral envelope glycoprotein indicates that said glycoprotein is displayed or detectable on the external surface of the viral particles in question. For example, viral particles decorated with VSV-G include VSV particles (that are naturally composed of VSV-G) as well as VSV-G-pseudotyped particles (such as those corresponding to VSV-G-pseudotyped retroviral and lentiviral vectors described herein).

The terms “pseudotype” or “pseudotyping” refers to the replacement of a component of a virus with that from a heterologous virus. In particular, “pseudotyping” denotes the formation of a recombinant virus whose viral envelope has been modified to include envelope proteins of another virus, such as a vesiculovirus envelope glycoprotein (G) as disclosed herein, resulting in modified tropism. For example, in VSV-G-pseudotyped lentiviral and retroviral vectors the viral particles comprise a VSV-G envelope protein (e.g. a naturally-occurring VSV-G or a modified VSV-G as disclosed herein). In such vectors VSV-G replaces the original envelope protein. Thus, VSV-G-pseudotyped vectors are typically prepared by allowing the VSV-G protein to be present during viral production. Viral particles produced in packaging cells (e.g. HEK293 cells) can be pseudotyped with VSV-G by expressing VSV-G in these cells. This can be facilitated by, for example, transfection of a VSV-G expression vector, or induced expression of the VSV-G gene integrated into the host's chromosomal DNA. An example of a VSV-G pseudotyped lentiviral vector is the product rLV.EF1.ZsGreen1-9, Cat. #0038VCT, provided by TaKaRa Bio.

In another embodiment, the viral particle is of an oncolytic virus (OV, e.g., an oncolytic vesiculovirus). In another embodiment, said viral particle is a retroviral particle (e.g., a recombinant lentiviral particle). In another embodiment, the viral particle (e.g., a vesiculovirus or a recombinant vector derived therefrom) encodes an adapter molecule of the invention. In another embodiment, the viral (e.g., lentiviral) particle further encodes a CAR or a therapeutic agent.

In another embodiment, the therapeutic agent is a human cystic fibrosis transmembrane conductance regulator (CFTR) polypeptide, or a therapeutic fragment thereof. In another embodiment, the therapeutic agent is a synthetic complementary guide RNA (gRNA) directed to a gene involved in a monogenic inherited disorder or a malignancy. In another embodiment, the therapeutic agent is the gene of Adenosine Deaminase, the Survival of Motor Neuron 1 (SMN1) gene, the gene of Hemoglobin subunit beta, the ABCD1 gene, the gene of Aryl sulfatase A, the ARPC1B gene and the like. In another embodiment, said therapeutic agent is a gene therapy agent selected from the group consisting of ADA, Hemoglobin subunit beta, ABCD1, Aryl sulfatase A, ARPC1B, IL2RG, WAS, CYBB, CD18, DCLRE1C, FANCA, PKLR, IDUA, CTNS, and GLA gene products. Each possibility represents a separate embodiment of the invention.

### Chimeric Antigen Receptors (CAR)

The terms “chimeric antigen receptor” and “CAR” are used herein interchangeably and refer to engineered recombinant polypeptide or receptor which are grafted onto cells and comprising at least (1) an extracellular domain comprising an antigen-binding region, e.g., a single chain variable fragment of an antibody or a whole antibody, (2) a transmembrane domain to anchor the CAR into a cell, and (3) one or more cytoplasmic signaling domains (also referred to herein as “an intracellular signaling domains”). The extracellular domain comprises an antigen binding domain (ABD) and optionally a spacer or hinge region. The antigen binding domain of the CAR targets a specific antigen. The targeting regions may comprise full length heavy chain, Fab fragments, or single chain variable fragment (scFvs). The antigen binding domain can be derived from the same species or a different species for or in which the CAR will be used in. In one embodiment, the antigen binding domain is scFv.

The extracellular spacer or hinge region of a CAR is located between the antigen binding domain and a transmembrane domain. Extracellular spacer domains may include, but are not limited to, Fc fragments of antibodies or fragments or derivatives thereof, hinge regions of antibodies or fragments or derivatives thereof, constant domains such as CH2 region or CH3 region of antibodies, accessory proteins, artificial spacer sequences or combinations thereof. The term “transmembrane domain” refers to the region of the CAR, which crosses or bridges the plasma membrane. The transmembrane domain of the CAR of the invention is the transmembrane region of a transmembrane protein, an artificial hydrophobic sequence or a combination thereof. The term “intracellular domain” refers to the intracellular part of the CAR and may be an intracellular domain of T cell receptor or of any other receptor (e.g., TNFR superfamily member) or portion thereof, such as an intracellular activation domain (e.g., an immunoreceptor tyrosine-based activation motif (ITAM)-containing T cell activating motif), an intracellular costimulatory domain, or both.

The term “antigen binding portion”, “antigen binding region” and“antigen binding domain” are used herein interchangeably and refer to one or more fragments of an antibody that retain the ability to specifically bind to an antigen. It has been shown that the antigen binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb, which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). Such single chain antibodies are also intended to be encompassed within the term “antigen binding portion” of an antibody. In certain embodiments of the invention, scFv molecules are incorporated into a fusion protein. According to some embodiments, the antigen binding domain is a scFv. According to some embodiments, the antigen binding domain of the present invention binds specifically to a tumor associated antigen.

The term “tumor associated antigen” (TAA) or “tumor antigen” as used herein refers to any antigen which is found in significantly higher concentrations in or on tumor cells than on normal cells. According to some embodiments, the tumor associated antigen is selected from AFP, ALK, B7H3, BAGE protein, BCMA, BIRC5, BIRC7, β-catenin, -8 brc-ab1, BRCA1, BORIS, CA9, CA125, carbonic anhydrase IX, caspase 1, CALR, CCR5, CD19, CD20, CD22, CD24, CD30, CD33, CD38, CD40, CD123, CD133, CD138, CDK4, CEA, Claudin 18.2, cyclin-B1, CYP1B1, EGFR (Epidermal Growth Factor receptor), EGFRvIII, ErbB2/Her2 (Epidermal growth factor Receptor 2), ErbB3, ErbB4, ETV6-AML, EpCAM (Epithelial Cellular Adhesion Molecule, e.g., UniProt: P16422), EphA2, Fra-1, FOLR1, GAGE, GD2, GD3, GloboH, phosphatidylinositol proteoglycan-3, GM3, gp100, HLA/B-raf-kinases, HLA/k-ras, HLA/MAGE-A3, hTERT, IGF-1R, IL13R α2. LMP2 k-Light, LewisY, MAGE, MART-1, Mesothelin (e.g., UniProt Q13421), ML-IAP, MOv-γ, Muc1 (Mucin 1; Carcinoma-associated mucin; e.g., UniProt: P15941), Muc2, Muc3, Muc4, Muc5, CA-125, MUM1, NA17, NKG2D, NY-BR1, NY-BR62, NY-BR85, NY-ESO1, OX40, p15, p53, PAP, PAX3, PAX5, PCTA-1, PLAC1, PRLR, PRAME, PSMA, RAGE protein, Ras, RGS5, Rho, ROR1, SART-1, SART-3, STEAP1, STEAP2, TAG-72, TGF-β, TMPRSS2, soup-antigen, TRP-1, TRP-2, tyrosinase, urea soluble protein-3 and 5T4. According to other exemplary embodiments, said TAA may be IGF-1R (Insulin-like Growth Factor 1 Receptor; e.g., UniProt: P08069) or PSCA (Prostate Stem Cell Antigen; e.g., UniProt: 042653). Each possibility represents a separate embodiment of the invention. TAA may be (nearly) exclusively associated with a tumor or tumor cell(s) and not with healthy normal cells or may be over-expressed (e.g., 2 times, 5 times, 10 times, 50 times, 100 times, 1000 times or more) in a tumor tissue or tumor cell(s) compared to healthy normal tissue or cells.

The terms “binds specifically”, “selectively binds” or “specific for” with respect to an antigen-binding domain of an antigen-binding molecule such as an antibody, or of a fragment thereof or of a CAR refers to an antigen-binding domain which recognizes and binds to a specific antigen, but does not substantially recognize or bind other molecules in a sample. The term encompasses that the antigen-binding domain binds to its antigen with high affinity and binds other antigens with low affinity. An antigen-binding domain that binds specifically to an antigen from one species may bind also to that antigen from another species. This cross-species reactivity is not contrary to the definition of that antigen-binding domain as specific. An antigen-binding domain that specifically binds to an antigen may bind also to different allelic forms of the antigen (allelic variants, splice variants, isoforms etc.). This cross reactivity is not contrary to the definition of that antigen-binding domain as specific.

In another embodiment, the CAR is directed to a hematopoietic tumor antigen, e.g., CD19 or CD20. In a particular embodiment, the CAR is directed to CD19. In another embodiment, said CAR contains a T cell receptor-zeta (TCR-zeta) signal transduction domain with the CD28 and/or CD137 (4-1BB) intracellular domains in tandem. For example, without limitation, the CAR may be an anti-CD19-BBζ CAR (anti-CD19 scFv, Milone, M C. et al, 2009, Molec. Therapy, 17, 1453-1464), Hu19-CD828Z (anti-CD19 scFv, GenBank accession No. QHQ73568.1) or Hu20-CD8BBZ (anti-CD20 scFv, GenBank accession No. WBR62865.1).

### Gene Editing

The clustered regularly interspaced short palindromic repeats (CRISPR) and CRISPR-associated protein (Cas) system has recently evolved to be a revolutionary platform for both genome-editing manipulations in a broad range of tissues and organs. CRISPR/Cas has become a valuable and effective option for the treatment of many diseases and disorders which would otherwise be incurable. The most studied and developed system of CRISPR/Cas is the CRISPR-associated enzyme Cas9. Cas9 is capable of binding DNA only in the presence of a specific sequence, known as a protospacer-adjacent motif (PAM). The recognition of the PAM motif enables Watson-Crick RNA-DNA base pairing with synthetic complementary guide RNA (gRNA). Following assembly of the ternary complex between gRNA, DNA, and Cas9, the endonuclease domains of Cas9 become active, and cleave both DNA strands, which results in the formation of double-strand DNA breaks. The unprecedented efficiency, accuracy, and specificity of the Cas9 protein has been rapidly recognized and directed towards a wide range of genome-editing applications, from basic science to translational and clinical research and medicine (Dong, et al., Current State and Perspectives. Viruses, 2021. 13 (7)).

Generally, the most common genetic variants associated with hereditary diseases in humans are point mutations and functional single-nucleotide polymorphisms (SNPs). Cas9 fused with APOBEC1 enables C-to-T conversion in a 6-nucleotide window. Similarly, A-to-G systems were developed. More advanced tools were developed as well, and shown to correct mutations leading to sickle cell disease and Tay-Sacs in human cells in culture. VSV-G-pseudotyped LVVs are one of the main delivery vehicles for the CRISPR/Cas systems due to their ability to carry and deliver bulky and complex transgenes and sustain robust and long-term expression in a broad range of dividing and non-dividing cells in vitro and in vivo. Improved target cell specificity of LVVs will greatly advance CRISPR/Cas-based gene therapy, particularly in vivo. As used herein, the term “gRNA” refers to a piece of RNAs that function as guides for RNA- or DNA-targeting enzymes, which they form complexes with. For example, gRNA can be designed to be used for targeted editing, such as with CRISPR-Cas9. The targeting specificity of CRISPR-Cas9 is determined by a short sequence (e.g., 20-nt) at the 5′ end of the gRNA. The desired target sequence must precede the PAM. After base pairing of the gRNA to the target, Cas9 mediates a double strand break about 3-nt upstream of PAM. In other embodiments, gRNA targets within a gene of interest may be determined using a variety of publicly available bioinformatic tools including the CHOPCHOP algorithm, Broad Institute GPP, CasOFFinder, CRISPOR, Deskgen, etc. Methods for evaluating the efficacy of the nucleic acid agents and modulators include, for example, DNA sequencing, PCR, RT-PCR, RNase protection, in-situ hybridization, primer extension, Southern blot, Northern Blot and dot blot analysis.

### Recombinant Methods

Polypeptides, peptides and nucleic acid molecules may conveniently be produced by recombinant technology. Recombinant methods for designing, expressing and purifying proteins, peptides and nucleic acid molecules are known in the art (see, e.g., Sambrook et al., 2001, Molecular Cloning: A Laboratory Manual, Cold Springs Harbor Laboratory, New York). Nucleic acid molecules may include DNA, RNA, or derivatives of either DNA or RNA. An isolated nucleic acid sequence encoding a polypeptide or peptide can be obtained from its natural source, either as an entire (i.e., complete) gene or a portion thereof. A nucleic acid molecule can also be produced using recombinant DNA technology (e.g., polymerase chain reaction (PCR) amplification, cloning) or chemical synthesis. Nucleic acid sequences include natural nucleic acid sequences and homologs thereof, including, but not limited to, modified nucleic acid sequences in which nucleotides have been inserted, deleted, substituted, and/or inverted in such a manner that such modifications do not substantially interfere with the nucleic acid molecule's ability to encode a functional product. A polynucleotide or oligonucleotide sequence can be deduced from the genetic code of a protein, however, the degeneracy of the code must be taken into account, as well as the allowance of exceptions to classical base pairing in the third position of the codon, as given by the so-called “Wobble rules”. Polynucleotides that include more or less nucleotides can result in the same or equivalent proteins. Using recombinant production methods, selected host cells, e.g., of a microorganism such as E. coli or yeast, are transformed with a hybrid viral or plasmid DNA vector including a specific DNA sequence coding for the polypeptide and the polypeptide is synthesized in the host upon transcription and translation of the DNA sequence.

Such recombinant methods may also be used in the preparation of nucleic acid constructs, including in particular expression constructs or vectors used for delivering and expressing the adapters of the invention in suitable expression systems. The constructs comprise nucleic acid molecules of the invention, and may also comprise regulatory sequences or selectable markers, as known in the art. The nucleic acid construct (also referred to in some embodiments as a vector) may include additional sequences that render this vector suitable for replication and integration in prokaryotes, eukaryotes, or optionally both (e.g., shuttle vectors). In addition, a typical cloning vector may also contain transcription and translation initiation sequences, transcription and translation terminators, and a polyadenylation signal.

### Pharmaceutical Compositions

In another aspect, there is provided a pharmaceutical composition, comprising a therapeutically effective amount of the adapter-modified viral particles. In another embodiment the pharmaceutical composition further comprises a pharmaceutically acceptable carrier, excipient or diluent. In accordance with this invention, the term “pharmaceutical composition” relates to a composition for administration to a patient, preferably a human patient. Pharmaceutical compositions of the invention comprise a therapeutically effective amount of an active ingredient as disclosed herein (e.g., a nucleic acid construct or a vector, viral particle or cell comprising said nucleic acid construct) and at least one pharmaceutically acceptable excipient or carrier. In some embodiments, the pharmaceutical composition comprises a composition for parenteral, transdermal, intraluminal, intravenous, intra-arterial, or intrathecal administration or by direct injection into the tissue or tumor. It is in particular envisaged that said pharmaceutical composition is administered to a patient via infusion or injection. Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well-known methods.

In another aspect, there is provided a pharmaceutical composition comprising a therapeutically effective amount of adapter-modified viral particles and a pharmaceutically acceptable carrier, excipient or diluent, the particles comprising:
(i) an adapter molecule, comprising an anchoring component covalently linked by a flexible linker to a targeting component, wherein:
   a. the anchoring component comprises: at least one isolated CR motif selected from the group consisting of: hLDLR Class-A repeat 2 (hLDLR CR2), hLDLR CR3, and homologs thereof.
   b. the flexible linker comprises at least five contiguous amino acid residues selected from the group consisting of glycine, serine and/or alanine,
   c. the targeting component comprises: a ligand of a receptor expressed preferentially on the surface of a mammalian target cell, or an antigen-binding molecule that selectively binds the receptor, and
(ii) particles of a virus or viral vector decorated with a vesiculovirus envelope glycoprotein (G) selected from the group consisting of: vesicular stomatitis virus (VSV)-G, Cocal virus (COV)-G and Maraba virus (Maraba)-G,
wherein the vesiculovirus envelope glycoprotein is specifically complexed in a non-covalent manner with the anchoring component of the adapter molecule of (i).

An “effective amount” or “therapeutically effective amount” refers to an amount sufficient to exert a beneficial outcome in a method of the invention. More specifically, a therapeutically effective amount means an amount of active ingredients effective to prevent, alleviate, or ameliorate symptoms of a disorder (e.g., cancer or genetic disorder) or prolong the survival of the subject being treated. Determination of a therapeutically effective amount is well within the capability of those skilled in the art, especially in light of the detailed disclosure provided herein. For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans. Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition (See, e.g., Fingl, E. et al. (1975), “The Pharmacological Basis of Therapeutics,” Ch. 1, p. 1.).

In another embodiment, the composition further comprises a proprotein convertase subtilisin/kexin type-9 (PCSK9) polypeptide. Additionally or alternatively, said composition further comprises said adapter molecule at an additional amount in excess of said viral particles. In another embodiment the vesiculovirus envelope glycoprotein is complexed non-covalently, specifically and stably with the anchoring component of said adapter molecule under physiological conditions in vivo. As used herein, the term “proprotein convertase subtilisin/kexin type-9”, “PCSK9” or “subtilisin/kexin type 9” refers to an enzyme encoded in humans by the PCSK9 gene on chromosome 1. PCSK9 (also known as FH3, HCHOLA3, NARC-1, and NARC1) is the ninth member of the proprotein convertase family of proteins that activate other proteins. The term “PCSK9” denotes both the proprotein and the product generated following autocatalysis of the proprotein.

The circulating PCSK9 is a soluble member of the mammalian proprotein convertase family of secretory serine endoproteases. PCSK9 is mainly synthesized and secreted from liver with lower levels of expression in the intestine, kidney and brain. Following cleavage of the signal peptide (amino-acids 1-31) it undergoes autocatalytic cleavage in the endoplasmic reticulum lumen, releasing an N-terminal pro-domain (amino acids 32-152). Mature PCSK9 remains noncovalently bonded to its inhibitory pro-domain and is enzymatically inactive because the pro-domain occupies the active site cleft of the protease and shields it from interacting with other substrates. This circulating enzymatically-inactive complex is present in human plasma at a broad (100-fold) range of concentrations with a median value of 0.5 μg/mL. It binds to the first epidermal growth factor-like repeat of LDLR on the cell surface. Upon co-internalization, bound PCSK9 inhibits endocytic recycling of LDLR, resulting in lysosomal degradation of both proteins, thereby lowering cell surface LDLR. Similarly, PCSK9 induces the degradation of other LDLR family members, including LRP1, LRP8 and VLDLR, all of which are possible entry ports of VSV.

Human PCSK9 is identified at UniProt: Q8NBP7. An exemplary PCSK9 polypeptide to be used in embodiments of the invention is the human PCSK9 having the amino acid sequence as set forth in SEQ ID NO: 41. In other embodiments, the use of PCSK9 homologs is contemplated, which homologs retain a high degree of homology (e.g., greater than 90%, 93%, 95% or 98%) with a naturally-occurring PCSK9, such that its ability to mediate specific LDLR-binding capacity is retained. In other embodiment, PCSK9 may be used in the form of a modified PCSK9 conjugated with a serum half-life elongating substance (e.g., PEG or immunoglobulin (Ig) fusion partners).

In one embodiment, the pharmaceutical composition comprises the PCSK9 polypeptide of (iii) and the adapter molecule of (iv). In another embodiment, the pharmaceutical composition comprises the PCSK9 polypeptide of (iii) at an amount effective to provide a blood concentration of 0.01-0.1 μM upon administration to a subject in need thereof (e.g., 0.01-0.1, 0.1-1 or 0.05-0.5 μM), and/or the adapter molecule of (iv) at an amount effective to provide a blood concentration of 1-10 μg/mL upon administration to a subject in need thereof (e.g., 1-5, 5-10, 3-8, 2-4, 5-7 or 8-10 μg/mL). In another embodiment, the pharmaceutical composition comprises the PCSK9 polypeptide and the effective amount is 5-500 mg. e.g., 5-50, 50-500, 10-100 or 25-250 mg. In another embodiment the pharmaceutical composition comprises the adapter molecule of (iv) and the effective amount is 60-600 μg. e.g., 60-300, 300-600, 100-500, 200-400, 100-200 or 500-600 μg. Each possibility represents a separate embodiment of the invention.

The compositions of the invention may be administered locally or systematically. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, poly(oxyethylene), vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like.

It is envisaged that the pharmaceutical composition of the invention might comprise, in addition to the active ingredient as disclosed herein, additional active agents, depending on the intended use of the pharmaceutical composition. Such agents might be anti-cancer drugs, e.g., immunotherapeutic agents such as immune checkpoint inhibitors. The term “checkpoint inhibitor” refers to drugs (e.g., antibodies or small molecules) that target and antagonize, neutralize, or otherwise reduce the activity of immune inhibitory checkpoint molecules like PD-1, PD-L1, and CTLA-4. For example, adapter-modified viral particles of oncolytic viral agents in accordance with the invention may conveniently be co-formulated or co-administered with immune checkpoint inhibitors such as Pembrolizumab (directed to PD-1).

In another embodiment, said viral vector further encodes a chimeric antigen receptor (CAR), a gene therapy agent (GTA) or a gene editing agent. In another embodiment, the targeting component comprises an antigen-binding molecule that selectively binds to human CD8 or CD56, and said viral vector is a lentiviral or retroviral vector pseudotyped with said envelope glycoprotein, and encoding a CAR directed to a tumor-associated antigen (TAA). In another embodiment, said GTA is selected from the group consisting of: Cystic Fibrosis Transmembrane Conductance Regulator (CFTR), Adenosine Deaminase, Survival of Motor Neuron 1 (SMN1), Hemoglobin subunit beta, ABCD1, Aryl sulfatase A, and ARPC1B.

In another embodiment said GTA is human CFTR, said viral vector is a lentiviral or retroviral vector pseudotyped with said envelope glycoprotein and the targeting component of said adapter molecule comprises a CEACAM6- or CEACAM1-binding portion of hCEACAM8, or an antigen-binding portion of an antibody directed to hCEACAM6 or hCEACAM1. In another embodiment, said GTA is human CFTR, said viral vector is a lentiviral or retroviral vector pseudotyped with said envelope glycoprotein and the targeting component of said adapter molecule comprises a CEACAM1-binding portion of hCEACAM5. In some embodiments, pharmaceutical compositions comprising adapter-modified viral particles for the treatment of CF are conveniently formulated for administration by inhalation. For example, without limitation, such formulations may contain one or more surfactants that facilitate inhalation or absorption of the therapeutic agent and/or permeation enhancers that increase the permeability of mucosal cells and tissue to said agent. By means of non-limitative examples, formulations for inhalation in accordance with the invention may contain phospholipid glycerols such as dimyristol glycerol, lysophosphatidylcholine and derivatives thereof, or other excipients such as poly-L-arginines; fatty acids, such as lauric acid; transkarbam; ceremides and modified ceremides; bile salts such as deoxycholate, glycolate, cholate, taurocholate, taurodeoxycholate, and glycodeoxycholate; salts of fusidic acid such as taurodihydrofusidate; poly(oxyethylene) sorbitan such as TWEEN™ 20 and TWEEN™ 80; sodium lauryl sulfate, and the like. In an exemplary embodiment, said composition contains 0.1% lysophosphatidylcholine and CFTR-encoding adapter-modified viral particles as disclosed herein.

In other exemplary embodiments, pharmaceutical compositions for providing a GTA to an HSC target cell as disclosed herein is conveniently formulated for intravenous administration (e.g., by formulations for injection or infusion as disclosed herein). In another embodiment, said virus is an OV further encoding said adapter protein. In another embodiment, the OV is a vesiculovirus encoding said envelope glycoprotein, and the targeting component of said adapter molecule is directed to a TAA. In another embodiment the TAA is selected from the group consisting of human CEACAM6, human CEACAM1, human c-KIT, and human PSMA, wherein each possibility represents a separate embodiment of the invention. In yet another embodiment, said TAA is FOLR1. Such pharmaceutical compositions are conveniently formulated for intratumoral administration as further disclosed herein.

### Process

In another embodiment, there is provided a process for producing the pharmaceutical composition, comprising complexing viral particles with adapter molecules of the invention (so as to form adapter-modified viral particles as disclosed herein). In another embodiment, complexing is performed in vitro, by admixing said viral particles with said adapter molecules, as disclosed herein. In other embodiments, complexing is performed directly in the host cell in cell culture, wherein a viral vector encoding an adapter of the invention is produced in an appropriate (e.g., mammalian) expression system. In yet another embodiment, complexing is performed directly in the host cell in vivo, in which a viral vector encoding an adapter of the invention is administered to a subject to selectively transduce the relevant target cells in vivo, as will be discussed in greater detail below.

In another aspect, there is provided a process for producing the pharmaceutical composition, comprising contacting the particles of the virus or viral vector of (ii) as disclosed herein with adapter molecule of (i) as disclosed herein, so as to produce the adapter-modified viral particles. In another embodiment, the contacting is performed in vitro, by incubating said particles with said adapter molecules under conditions so as to allow specific non-covalent complexing of said particles with the anchoring component of said adapter molecule. For example, lentiviral particles encoding a therapeutic agent (e.g. a CAR or GTA) may be incubated for 15-60 minutes at room temperature (e.g. in Opti-MEM™ or other compatible culture media) to form the adapter-modified viral particles. Suitable incubation conditions are further described in the Examples section below. In another embodiment, said particles and said adapter molecule are expressed in a mammalian expression system and said contacting is performed in said expression system, for example, the sLDLR-CEACAM8 adapter may be produced and then secreted by VSV-infected cells in a suitable mammalian expression system (e.g. HEK293 cells). Formation of the VSV-adapter complex takes place in said system after release of the VSV and the adapter from the infected cells.

In another embodiment, the process further comprises admixing the particles of (ii) or the adapter-modified viral particles with said adapter molecule of (iv), so as to produce a pharmaceutical composition comprising said adapter-modified viral particles and an excess of adapter molecules that are not complexed with said viral particles. As disclosed herein, a pharmaceutical composition comprising adapter-modified viral particles and an excess of free (non-complexed) adapter molecules are produced using an amount of adapter molecules that is greater than that needed to suppress the original tropism of the virus below detectable levels. Thus, addition of the excess amount of adapter does not result in a substantial (detectable) specific binding to the desired target cell upon a short-term in vitro incubation (up to 1 hour, e.g., AsPC-1 cells: about 15 minutes), as evaluated e.g., by altered tropism or binding of labeled free ligand. Yet, such pharmaceutical compositions are as disclosed herein to provide improved long-term efficacy and selectivity in vivo (e.g., for a period of hours, e.g., within 1-4 hours of administration). For example, excess adapter compositions providing a concentration of 1-5 μM upon administration as disclosed herein may advantageously be used. Additionally or alternatively, said process may further comprise admixing the particles of (ii) or the adapter-modified viral particles with said PCSK9 polypeptide of (iii).

The obtained or purified complexes may be formulated (alone or in combination with the PCSK9 polypeptide and/or excess of adapter molecules) in the form of a pharmaceutical composition, e.g., a composition for intravenous or intratumoral injection or for administration by inhalation as disclosed herein.

### Cell Compositions

In another embodiment, there is provided a cell composition, comprising a mammalian (typically human) cell population transduced by a virus, viral vector or adapter-modified viral particles of the invention as disclosed herein. In one embodiment, the cell population is an immune cell population (comprising e.g., T cells and/or NK cells). In a particular embodiment, the cell composition is an adoptive transfer cell composition (ACT). For example, adoptive transfer cell compositions to be used in embodiments of the invention may contain effector cells (e.g., CD8⁺ T cells or CD56+NK cells), transduced with adapter-modified viral particles comprising: a vesiculo-G protein-pseudotyped retroviral (e.g., lentiviral) vector encoding a tumor-specific CAR or TCR, the vector complexed with adapter molecules as disclosed herein in which the targeting component comprises a DARPin, antibody or fragment thereof directed to a surface-expressed receptor on the immune effector cells (e.g., CD8 or CD56, respectively).

The term “cell composition” as used herein indicates a pharmaceutical composition that contains cells or cellular material as the active ingredient. Cell compositions typically contain pharmaceutically acceptable carriers, excipients or diluents, and optionally additional components other than cells such as culture medium or preservation liquid. As used herein, and unless otherwise specified, the term “adoptive transfer” refers to a form of passive immunotherapy where previously sensitized immunologic agents (e.g., cells or serum) are transferred to the recipients. The phrases “adoptive cell transfer”, “adoptive transfer immunotherapy”, “adoptive transfer therapy”, “adoptive cell therapy” and “adoptive cell immunotherapy” are used interchangeably herein to denote a therapeutic or prophylactic regimen or modality, in which effector immunocompetent cells are administered (adoptively transferred) to a subject in need thereof, to alleviate or ameliorate the development or symptoms of the disorder (e.g., cancer or infectious diseases). Thus, an ACT composition in accordance of the invention typically contains effective amounts (e.g., at least 5×10⁶ cells and up to about 10×10⁹ cells), which are produced under sterile and suitable (e.g., cGMP grade) conditions.

In some embodiments, the cell composition is amenable for cancer immunotherapy. The term “cancer immunotherapy” refers to treatment of a subject afflicted with, or at risk of suffering a recurrence of cancer, by a method comprising modulating an immune response in the subject. In particular, cancer immunotherapies are typically aimed at inducing and/or stimulating the immune response of the subject towards cancer cells. Protocols for generating ACT compositions are well known in the art. For example, compositions for adoptive cell transfer may be prepared by methods including activating a T cell population by a TCR stimulation, and expansion of the cells to obtain a therapeutically effective amount of effector T cells for administration. Such methods include but are not limited to, Rapid Expansion Protocols (REP). In various embodiments, the TCR stimulation may be antigen non-specific (performed, for example, using antibodies specific to CD3 that activate the receptor upon binding, e.g., OKT3) or antigen-specific (using suitable antigen presenting cells and antigen). In the context of cancer treatment, antigen-specific stimulation typically employs stimulation to tumor-associated antigens.

In some embodiments, one commonly used approach for stimulating proliferation, in particular of CD8⁺ T cells, is the incubation of T cells with soluble anti-CD3 antibody in the presence of Fc receptor-bearing accessory cells (feeder cells), an approach designated the REP. Antibody “presented” to T cells in this manner generates a more effective proliferative signal than soluble anti-CD3 alone or anti-CD3 immobilized on a plastic surface. In the treatment of cancer, adoptive cell therapy typically involves collecting T cells that are found within the tumor of the patient (referred to as tumor-infiltrating lymphocytes, TIL), which are encouraged to multiply ex vivo using high concentrations of IL-2, anti-CD3 and allo-reactive feeder cells. These T cells are then transferred back into the patient along with exogenous administration of IL-2 to further boost their anti-cancer activity.

Exemplary adapters, constructs and compositions for preparing ACT compositions amenable for cancer immunotherapy are disclosed and exemplified herein. For example, for the generation of ACT compositions comprising modified CD8⁺ cells as the active ingredient, an adapter in which the targeting component is DARPin53F6 (SEQ ID NO: 11) and the anchoring component is sLDLR₍₂₅₋₁₄₅₎ (SEQ ID NO: 5) may be used, e.g., complexed with a VSV-G pseudotyped lentiviral or retroviral vector encoding a CAR directed to a TAA (e.g., CD19). In another particular embodiment, the cell composition to be used in embodiments of the invention may contain hematopoietic stem cells (HSC, e.g., CD34⁺ cells), transduced with adapter-modified viral particles comprising: a vesiculo-G protein-pseudotyped retroviral (e.g., lentiviral) vector encoding a gene intended for complementing a defective inherited gene, e.g., adenosine deaminase, the vector complexed with adapter molecules as disclosed herein in which the targeting component comprises a DARPin, antibody or fragment thereof, directed to a surface-expressed receptor on the immune effector cells (e.g., CD34 or CD133, respectively).

In other embodiments, ligands of receptors expressed preferentially on HSC following mobilization from the bone marrow to peripheral blood (e.g., by treating the HSC donor with G-CSF), for example folic acid (directed to FOLR1), or, in other embodiments, SCF (targeting c-KIT) or FLT3L (targeting FLT3) may be used as targeting components. For example, for the generation of cell compositions comprising modified HSC, as the active ingredient, an adapter in which the targeting component is folic acid and the anchoring component is sLDLR₍₂₅₋₁₄₅₎ (SEQ ID NO: 5) may be used, e.g., complexed with a VSV-G pseudotyped lentiviral or retroviral vector encoding a GTA. Exemplary GTA for administration into HSC for treating suitable inherited monogenic disorders (e.g., immune deficiencies, blood disorders and lysosomal storage diseases) are identified in Table 2 below.

As used herein, the term “hematopoietic stem cells” (“HSC”) refers to immature blood cells having the multipotential capacity to self-renew and to differentiate into mature blood cells containing diverse lineages, including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34⁺ cells, which are immature cells (or HSC) that express the CD34 cell surface marker. CD34 is a marker of human HSC, and the colony-forming activity of human bone marrow (BM) cells is found in the CD34⁺ fraction. In humans, CD34⁺ cells are believed to include a subpopulation of cells with the stem cell properties defined above, whereas in mice, HSC are CD34⁻. Human HSCs are readily obtainable from available sources, including human umbilical cord blood, adult bone marrow and peripheral blood. According to certain advantageous embodiments, the use of G-CSF-activated HSC is contemplated, either ex vivo or by transfusion into athymic mice. Such cells are collected following mobilization of bone-marrow derived HSC to the blood circulation prior to their collection by apheresis (for example, using sub-cutaneous administration of 10-16 μg/Kg G-CSF, daily for 4 days prior to leukapheresis).

In another embodiment, there is provided a process for producing a cell composition of the invention, comprising transducing the cell population with adapter-modified viral particles as disclosed herein.

### Therapeutic Use

In another aspect, adapter molecules of the invention are for use in delivering a virus or viral vector selectively into a mammalian target cell in vivo. In another embodiment, the use comprises complexing said virus or viral vector with an adapter molecule of the invention so as to form adapter-modified viral particles as disclosed herein, and administering the resulting adapter-modified viral particles to said subject. In another aspect, there is provided a method of delivering a virus or viral vector selectively into a mammalian (e.g., human) target cell in vivo, comprising complexing said virus or viral vector with an adapter molecule of the invention so as to form adapter-modified viral particles as disclosed herein, and administering the resulting adapter-modified viral particles to said subject.

In another embodiment, the adapter-modified viral particles of the invention are for use in concurrent or sequential combination with a PCSK9 polypeptide. In another embodiment, the use comprises administering to said subject a pharmaceutical composition comprising a PCSK9 polypeptide prior to and/or concomitantly with administration of the adapter-modified viral particles. In another embodiment, the methods of the invention further comprise administering to said subject a pharmaceutical composition comprising a PCSK9 polypeptide prior to and/or concomitantly with administration of the adapter-modified viral particles. In another embodiment, the PCSK9 polypeptide is administered at an amount and under conditions so as to downregulate the surface expression of LDLR family receptors in said subject.

In some embodiments, the effective amount of an auxiliary agent (e.g., a PCSK9 polypeptide or a non-complexed adapter) to be used in the compositions and methods of the invention is an amount effective to provide predefined blood concentrations upon administration to a subject in need thereof. For example, when treating an adult human subject, an effective amount of 5-500 mg PCSK9 may provide a blood concentration of 0.01-0.1 μM upon administration. In another example, an effective amount of 60-600 μg non-complexed adapter molecules may provide a blood concentration of 1-10 μg/mL (or 1-5 μM) upon administration to said subject. Additional dosage regimes can be determined by the treating physician based on the characteristics of the subject and the condition to be treated.

In other aspects, the adapter-modified viral particles, pharmaceutical compositions and cell compositions of the invention are for use in treating a disease or condition in a subject in need thereof, wherein each possibility represents a separate embodiment of the invention. In another aspect, the invention provides a method of treating a disease or condition in a subject in need thereof, comprising administering to said subject a pharmaceutical composition or cell composition of the invention.

In various embodiments, the disease or condition may be e.g., a tumor, an inherited monogenic disorder, or a genetic respiratory condition, wherein each possibility represents a separate embodiment of the invention.

In some embodiments, the disease or condition to be treated by the compositions and methods of the invention is a hyperproliferative disorder, in particular a tumor. As used herein, the terms “cancer” and “tumor” are used interchangeably and include both solid tumors and hematopoietic tumors. In some embodiments, the tumor is a malignant tumor. In other embodiments, the tumor is metastatic. Exemplary tumors to be treated include, but are not limited to, a hematological tumor, a lung tumor, a prostate tumor, a breast tumor, a gynecological tumor, a pancreatic tumor and malignant glioma. Each possibility represents a separate embodiment of the invention.

In certain exemplary embodiments, said tumor expresses a TAA selected from the group consisting of: PSMA, c-KIT, FOLR1, HER2, CEACAM6 and CEACAM1, wherein each possibility represents a separate embodiment of the invention. In certain exemplary embodiments, said TAA is selected from the group consisting of: PSMA, c-KIT, FOLR1, CEACAM6 and CEACAM1. In certain exemplary embodiments, said TAA is selected from the group consisting of: PSMA, FOLR1, CEACAM6 and CEACAM1. In a particular embodiment, said tumor is a PSMA⁺ tumor, e.g., a prostate tumor. In another embodiment, said tumor is a CEACAM6⁺ and/or CEACAM1⁺ tumor, e.g., a pancreatic tumor. In another particular embodiment, said tumor is a c-KIT⁺ tumor, e.g., a hematolpoietic tumor such as an acute myeloid leukemia (AML).

For example, for treatment of a tumor, the viral particles may be complexed with an adapter of the invention in which the cell-targeting component specifically binds to a tumor-associated antigen (typically a surface-expressed receptor facilitating receptor-mediated endocytosis of the bound ligand), and facilitate specific viral oncolysis. In another exemplary embodiment relating to tumor treatment, the viral particles may be complexed with an adapter of the invention in which the cell-targeting component specifically binds to a surface-expressed receptor on an immune cell, to generate genetically manipulated tumor-specific immune cells facilitating anti-tumor immunity.

In some embodiments, viral particles of the invention comprise an oncolytic virus. As used herein, the term “oncolytic virus” (or OV) refers to a virus capable of selectively replicating in a cancer cell, and slowing the growth or inducing the death of the cancer cell, either in vitro or in vivo, while having no or minimal effect on normal cells. In certain embodiments, the OV spread within a tumor without causing damages to non-cancerous tissues. In certain embodiments, the OV do not replicate or replicate at a reduced speed in non-cancer cells as compared to in cancer cells. An OV can occur naturally or can be a genetically modified virus (also referred to herein as a viral vector or a recombinant viral vector). Non-limiting exemplary OV include, Maraba viruses, vesicular stomatitis viruses (VSV, including vesicular stomatitis Indiana virus strains), and Cocal viruses.

According to advantageous embodiments of the invention, said virus further encodes the adapter molecule. Upon propagation of such recombinant viruses in the target tumor cells, their progeny may be complexed with the adapter directly upon budding from the virus-infected cells, thereby directing the progeny viruses to the tumor cells as well.

In another embodiment, the targeting component of said adapter molecule binds to hCEACAM6. For example, the adapter may contain the human proLDLR₍₂₅₋₁₄₅₎ (containing CR1, CR2 and CR3, SEQ ID NO: 5) anchoring component, fused at its C-terminus through a flexible peptide linker to the N-terminal domain of pro-carcinoembryonic antigen cell-adhesion molecule 8 (pro-CEACAM8₍₃₅₋₁₄₀₎, SEQ ID NO: 12) as the targeting component. In another exemplary embodiment, said adapter may contain a monoclonal antibody or fragment thereof directed against human CEACAM6 as the targeting component. As demonstrated and exemplified herein, complexes of adapters of the invention directed to human CEACAM6 (exemplified by the sLDLR-CEACAM8 adapter, see Examples 3-9 and 13 below) with viruses decorated with vesiculo-G Proteins (exemplified by VSV and recombinant VSV vectors encoding the adapter), may be used for selective viral oncolysis of tumor cells expressing high levels of CEACAM6 and/or CEACAM1. A non-limitative example of such tumor cells are pancreatic tumor cells expressing high levels of CEACAM6.

In another embodiment, the targeting component of said adapter molecule binds to the human SCF receptor c-KIT. For example, the adapter may contain the human proLDLR₍₂₅₋₁₄₅₎ anchoring component, fused at its C-terminus through a flexible peptide (or PEG) linker to a soluble receptor-binding fragment of human c-KIT ligand, also termed SCF (pro-huKITL₍₂₆₇₋₇₆₃₎. SEQ ID NO: 14) as the targeting component. In another exemplary embodiment, said adapter may contain a monoclonal antibody or fragment thereof directed against human c-KIT as the targeting component. As further demonstrated and exemplified herein, complexes of adapters of the invention directed to human c-KIT with viruses decorated with vesiculo-G proteins (exemplified by VSV and recombinant VSV vectors encoding the adapter), may be used for selective viral oncolysis of tumor cells expressing c-KIT. Non-limitative examples of said tumor cells are breast tumor cells, small-cell lung cancer cells, gynecological tumors, and malignant glioma.

In another embodiment, the targeting component of said adapter molecule binds to human PSMA. For example, the adapter may contain the human proLDLR₍₂₅₋₁₄₉₎ anchoring component, covalently linked with a human PSMAL as the targeting component. In another exemplary embodiment, said adapter may contain the human proLDLR₍₂₅₋₁₄₅₎ anchoring component, and a monoclonal antibody or fragment thereof directed against human PSMA as the targeting component. In another exemplary embodiment, said adapter may contain the human proLDLR₍₂₅₋₁₄₅₎ anchoring component, and a DARPin selected for very high affinity towards human PSMA as the targeting component. As further demonstrated and exemplified herein, complexes of adapters of the invention directed to the human PSMA (exemplified by the sLDLR-PSMAL adapter, see Example 12 below) with viruses decorated with vesiculo-G proteins (exemplified by VSV and recombinant VSV vectors encoding the adapter), may be used for selective viral oncolysis of tumor cells expressing PSMA. Non-limitative examples of said tumor cells are prostate cancer cells.

In another embodiment, the targeting component of said adapter molecule binds to the human FOLR1 receptor. For example, the ligand may be folic acid, which may be chemically derivatized to contain a flexible PEG chain as a linker. By means of a non-limiting example, the adapter may contain folic acid as a specific ligand, coupled through a flexible PEG linker to alpha amino and epsilon amino groups of the human proLDLR₍₂₅₋₁₄₅₎ anchoring component. In another exemplary embodiment, said adapter may contain a monoclonal antibody or fragment thereof directed against human FOLR1 as the targeting component. As further demonstrated and exemplified herein, complexes of adapters of the invention directed to human FOLR1 (exemplified by the FA-PEG-sLDLR adapter, see Example 22 below) with viruses decorated with vesiculo-G proteins (exemplified by VSV), may be used for selective viral oncolysis of tumor cells expressing FOLR1. Non-limitative examples of said tumor cells are cancers of the ovary, lung, endometrium, kidney, breast, bladder, and brain.

In other embodiments, the targeting component of said adapter molecule binds to human CD8 (hCD8). For example, the adapter may contain the human proLDLR₍₂₅₋₁₄₅₎ anchoring component, fused at its C-terminus through a flexible peptide linker to a DARPin specific to human CD8, e.g., DARPin 53F6. As further demonstrated and exemplified herein, adapters of the invention directed to hCD8, (exemplified by the sLDLR-53F6 adapter, see Examples 14-18 below) may be complexed with viral vectors decorated with vesiculo-G proteins (exemplified by LVVs encoding tumor-specific CAR) in vitro or in vivo, and used for treating the corresponding tumor in a subject in need thereof.

In another embodiment there is provided a method of generating CAR-T cells in vivo, the method comprising complexing an adapter that binds VSV-G with a VSV-G-pseudotyped vector encoding a tumor-specific CAR. In another embodiment, the adapter, vector and CAR are as disclosed herein.

In other embodiments, the disease or condition to be treated by the compositions and methods of the invention is an inherited monogenic disorder. A genetic disorder is a health problem caused by one or more abnormalities in the genome. When the genetic disorder is inherited from one or both parents, it is also classified as a hereditary (or inherited) disease or disorder. Monogenic disorders are a type of genetic disorders caused by mutation or alteration in the DNA sequence of a single gene. Inherited monogenic disorders are caused by the inheritance of single gene mutations; alternatively, a monogenic disorder arises as a consequence of a de novo mutation in either the paternal or maternal germ line. Examples of inherited monogenic disorders include without limitation genetic respiratory conditions, immune deficiencies, CNS disorders, blood disorders and lysosomal storage disorders, e.g., the disorders listed in Table 2 hereinbelow. Sequences of exemplary gene transcripts to be expressed in the respective target cells in order to treat each disease are identified in the corresponding UniProt numbers further listed in Table 2. The sequences of these GTA may be used in constructing viral vectors for treating the inherited monogenic disorders, by methods as disclosed and exemplified herein.

TABLE 2
Exemplary gene therapy agents (GTA) for inherited monogenic disorders

| Molecule | Full name & synonyms | Target cells | Disease/ category | UniProt |
|---|---|---|---|---|
| CFTR | Cystic fibrosis | Lung epithelia, | Cystic fibrosis | P13569 |
| | transmembrane | gastrointestinal | (Genetic respiratory conditions) | |
| | conductance regulator | epithelia | | |
| | ABCC7; MRP7 | | | |
| ADA | Adenosine deaminase | Hematopoietic | Adenosine deaminase deficiency; | P00813 |
| | Adenosine | stem cells | severe combined | |
| | aminohydrolase | (HSC) | immunodeficiency (ADA-SCID), | |
| | | | ADA deficiency | |
| | | | (Immune deficiencies) | |
| SMN1 | Survival of Motor Neuron | Horn cells of | Spinal muscular atrophy | Q16637 |
| | 1 | the spinal cord | (CNS disorders) | |
| Hemoglobin | Hemoglobin subunit beta | HSC | Beta-thalassemia; Sickle cell | P68871 |
| subunit beta | Hemoglobin beta chain | | disease | |
| | | | (Blood disorders) | |
| ABCD1 | ATP-binding cassette | HSC (young | Adrenoleukodistrophy (ALD) | P33897 |
| | sub-family D member 1 | patients) | (CNS disorders) | |
| Aryl | Aryl sulfatase A | HSC (young | Metachromatic Leukodystrophy | P15289 |
| sulfatase A | | patients) | (CNS disorders) | |
| ARPC1B | Actin Related Protein 2/3 | HSC | Immunodeficiency 71 with | O15143 |
| | Complex Subunit 1B | | inflammatory disease and | |
| | | | congenital thrombocytopenia | |
| | | | (IMD71) | |
| | | | (Immune deficiencies) | |
| IL2RG | Interleukin-2 receptor | HSC | X-linked severe combined | P31785 |
| | gamma chain | | immunodeficiency (SCID-X1) | |
| | | | (Immune deficiencies) | |
| WAS | WASP Actin Nucleation | HSC | Wiskott-Aldrich syndrome | P42768 |
| | Promoting Factor | | (WAS) | |
| | | | (Immune deficiencies) | |
| CYBB | Cytochrome B-245 Beta | HSC | X-linked chronic granulomatous | P04839 |
| | Chain | | disease (X-CGD) | |
| | | | (Immune deficiencies) | |
| CD18 | Integrin Beta 2, ITGB2 | HSC | leukocyte adhesion deficiency | P05107 |
| | | | (LAD) | |
| | | | (Immune deficiencies) | |
| DCLRE1C | DNA Cross-Link Repair | HSC | Artemis-deficiency severe | Q96SD1 |
| | 1C | | combined immunodeficiency | |
| | | | (ART-SCID) with sensitivity to | |
| | | | ionic radiation | |
| | | | (Immune deficiencies) | |
| FANCA | FA Complementation | HSC | Fanconi anaemia, | O15360 |
| | Group A | | complementation group A | |
| | | | (Blood disorders) | |
| PKLR | Pyruvate kinase L/R | HSC | pyruvate kinase deficiency of red | P30613 |
| | | | cells (PKRD) | |
| | | | (Blood disorders) | |
| IDUA | Alpha-L-Iduronidase | HSC | mucopolysaccharidosis 1H | P35475 |
| | | | (MPS1H), 1S (MPS1S) and 1H/S | |
| | | | (MPS1H/S) | |
| | | | (Lysosomal storage diseases) | |
| CTNS | Cystinosin, Lysosomal | HSC | Cystinosis (Several variants) | O60931 |
| | Cystine Transporter | | (Lysosomal storage diseases) | |
| GLA | Galactosidase Alpha; | HSC | Fabry disease | P06280 |
| | Alpha-D-Galactosidase A | | (Lysosomal storage diseases) | |

In other embodiments, the disease or condition to be treated by the compositions and methods of the invention is a genetic respiratory condition. The term genetic respiratory condition as used herein refers to a respiratory disease having a genetic origin. In particular, genetic respiratory conditions amenable for treatment by the compositions and methods of the invention include inherited monogenic disorders having pulmonary symptoms or manifestations, including, but not limited to airway disease, pulmonary parenchymal disease, and pulmonary vascular disease. A particularly advantageous genetic respiratory condition to be treated by the compositions and methods of the invention is CF.

In another embodiment, the adapter molecule of the invention is for use in delivering a virus or viral vector selectively into a target cell in a subject in need thereof, wherein the use comprises contacting particles of the virus or viral vector with said adapter molecule so as to produce adapter-modified viral particles, and administering the resulting adapter-modified viral particles to the subject. In another embodiment, said virus or viral vector is selected from the group consisting of: VSV, COV, Maraba, and viral vectors derived from vesiculovirus, retrovirus and lentivirus strains. As used herein, a vector “derived from” a particular viral strain is a recombinant viral vector in which the genetic material of the original virus had been modified (genetically engineered) by addition, deletion, replacement or otherwise modification of one or more genetic elements (e.g. open reading frames).

In another aspect, there is provided a method of delivering a virus or viral vector selectively into a target cell in a subject in need thereof, comprising contacting particles of the virus or viral vector with the adapter molecule as disclosed herein, so as to produce adapter-modified viral particles, and administering the resulting adapter-modified viral particles to the subject.

In another embodiment, said virus is selected from the group consisting of VSV, COV and Maraba viruses, or wherein said viral vector is selected from the group consisting of vesiculoviral and lentiviral vectors. In another embodiment, said target cell is selected from the group consisting of: a tumor cell, an immune cell, a HSC, and a lung epithelial cell. In another embodiment, the method is characterized by one of the following:
a) said target cell is a tumor cell and said receptor is selected from the group consisting of: PSMA, c-KIT, FOLR1, CEACAM6 and CEACAM1;
b) said target cell is an immune cell and said receptor is CD8 or CD56;
c) said target cell is a lung epithelial cell and said receptor is CEACAM6 or CEACAM1; or
d) said target cell is a HSC and said receptor is FOLR1.

In another embodiment, the method is characterized by one of the following:
a) said target cell is a PSMA+ tumor cell, said targeting component of said adapter is a selective PSMA ligand (PSMAL) comprising a Glu-NH—CO—NH-Lys pharmacophore or a GTI peptide (GTIQPYPFSWGY, SEQ ID NO: 37), and said virus or viral vector is an oncolytic vesiculovirus or a vesiculoviral vector further encoding said adapter molecule;
c) said target cell is a CD8⁺ immune cell, said targeting component of said adapter is DARPin53F6 (SEQ ID NO: 11), and said viral vector is a VSV-G pseudotyped lentiviral or retroviral vector encoding a CAR directed to a TAA;
d) said target cell is a lung epithelial cell, said targeting component of said adapter is CEACAM8₍₃₅₋₁₄₀₎ (SEQ ID NO: 12), and said viral vector is a VSV-G pseudotyped lentiviral or retroviral vector encoding a CFTR gene product; or
e) said target cell is a HSC, said targeting component of said adapter is folic acid, and said viral vector is a VSV-G pseudotyped lentiviral or retroviral vector encoding a GTA.

In another embodiment, said GTA is selected from the group consisting of ADA, Hemoglobin subunit beta, ABCD1, Aryl sulfatase A, ARPC1B, IL2RG, WAS, CYBB, CD18, DCLRE1C, FANCA, PKLR. IDUA, CTNS, and GLA gene products, wherein each possibility represents a separate embodiment of the invention.

According to certain advantageous embodiments, G-CSF is administered to a patient in need (for example, using sub-cutaneous administration of 10-16 μg/Kg G-CSF, daily for 4 days), thereby triggering release to the circulation of activated HSC. A viral particle encoding a GTA and complexed with an adapter specific for activated HSC, e.g., FA-PEG-sLDLR, is then administered to the patient by an intravenous route, enabling selective transduction of HSC in vivo.

In another aspect, the invention provides a method of treating a disease or condition in a subject in need thereof, comprising administering to the subject the pharmaceutical composition as disclosed herein. In another embodiment the disease or condition is an inherited monogenic disorder, and said composition comprises adapter-modified particles of a viral vector encoding a GTA. In another embodiment the disorder is cystic fibrosis, and the GTA is a hCFTR gene product. In another embodiment, said viral vector is a lentiviral or retroviral vector pseudotyped with said envelope glycoprotein and the targeting component of said adapter molecule comprises a receptor-binding portion of hCEACAM8, a receptor-binding portion of hCEACAM5, or an antigen-binding portion of an antibody directed to hCEACAM6. In another embodiment said adapter molecule is characterized in that said anchoring component consists essentially of sLDLR₍₂₅₋₁₄₅₎ (SEQ ID NO: 5), and said targeting component is CEACAM8₍₃₅₋₁₄₀₎ (SEQ ID NO: 12).

In another embodiment the disease or condition is a tumor. In another embodiment the tumor is selected from the group consisting of a hematological tumor, a lung tumor, a prostate tumor, a breast tumor, a gynecological tumor, a pancreatic tumor and malignant glioma, wherein Each possibility represents a separate embodiment of the invention. In another embodiment said virus is an OV further encoding said adapter molecule. In another embodiment said adapter molecule is characterized in that said anchoring component consists essentially of sLDLR₍₂₅₋₁₄₅₎ (SEQ ID NO: 5), and said targeting component is CEACAM8₍₃₅₋₁₄₀₎ (SEQ ID NO: 12). In another embodiment said tumor is a PSMA⁺ prostate tumor, and said targeting component of said adapter is a selective PSMA ligand (PSMAL) comprising a Glu-NH—CO—NH-Lys pharmacophore or a GTI peptide (GTIQPYPFSWGY, SEQ ID NO: 37). In another embodiment the targeting component of said adapter molecule comprises an antigen-binding molecule that selectively binds to human CD8 or CD56, and said viral vector is a lentiviral or retroviral vector pseudotyped with said envelope glycoprotein, and encoding a CAR directed to a TAA on said tumor. In another embodiment the method comprises administering said composition to said subject to thereby generate tumor-specific immune cells in vivo, or comprises incubating immune cells of a subject with said composition ex vivo to thereby generate tumor-specific immune cells, and re-introducing the resulting immune cells to said subject.

In another embodiment said adapter molecule is characterized in that said anchoring component consist essentially of sLDLR₍₂₅₋₁₄₅₎ (SEQ ID NO: 5), and said targeting component is DARPin53F6 (SEQ ID NO: 11). In another embodiment, said anchoring component in the methods of the invention is selected from the group consisting of: sLDLR₍₂₅₋₁₄₅₎ (SEQ ID NO: 5), sLDLR₂₅₋₁₄₉ (SEQ ID NO: 6) and sLDLR₂₅₋₁₈₇ (SEQ ID NO: 7). In another embodiment, the methods of the invention further comprise administering to said subject a PCSK9 polypeptide prior to and/or concomitantly with administration of said pharmaceutical composition, at a total dose of 5-500 mg per subject over a time period of 1-5 hours initiated at least one hour prior to administration of said pharmaceutical composition and maintained until administration of said pharmaceutical composition is completed, and/or further comprise administering to said subject a second pharmaceutical composition comprising said adapter molecule that is not complexed with viral particles at an effective amount of 60-600 μg.

According to other embodiments of the invention, one component of the adapter may be a polypeptide that binds reversibly to the viral surface proteins VSV-G, complement-resistant VSV-G, Cocal-G or Maraba-G. The second component, which is covalently-bound to the first one, may be selected from the following alternatives: a ligand of a specific cell surface receptor, or an antibody or antibody domain directed against a specific cell surface receptor, or a Designed Ankyrin Repeat Protein (DARPin) or LoopDARPin, selected for high-affinity binding to a specific cell-surface receptor or receptor-associated protein.

As disclosed herein, binding of adapters of the invention elicits receptor endocytosis, thereby leading to uptake of the adapter-bound vector. Examples of said cell surface receptors include EGF-receptor, ErbB2, HER2, CEA and CEACAM protein families, Emmprin, Endoglin, EpCAM, Folate Receptor (FOLR1), GRP 78, IGF-1R, Mesothelin, Muc-1, PSCA, c-KIT, PSMA, CD8, CD56 and other receptors as disclosed herein. Adapters of the invention may be used for directing viruses and viral vectors, as well as other synthetic vectors coated with said viral surface proteins, to specific cell types, characterized by expression of said cell surface receptors. Such viruses, viral vectors and synthetic vectors may be used for viral oncolysis, for gene therapy for generating CAR T cells and for introducing various agents into said cells in vivo and in vitro.

According to the one example of the present invention, the first component is a Class-A repeat of the LDLR or Class-A repeat of any other LDLR family member disclosed herein, which bind to VSV-G. Accordingly, adapters comprising said class A repeats (also referred to herein as Class A cysteine-rich repeat motifs (CR)), bind to vectors decorated with VSV-G, with complement-resistant VSV-G, with Cocal-G, or with Maraba-G (also referred to collectively as “vesiculo-G Proteins”). VSV is disclosed to bind through its VSV-G envelope protein to Class-A repeats 2 and 3 within the extracellular domain of human LDLR.

Hence, another aspect of the present invention provides an adapter whose first component consists of Class-A repeat 2 of human LDLR, Class-A repeat 3 of human LDLR, or any polypeptide containing Class-A repeat 2, Class-A repeat 3, or their combination. Binding of LDLR ligands such as LDL and Rhinovirus is reversible, as the cargo must be released following endocytosis. The free VSV-G is critical for delivering the genetic cargo into the cytoplasm as it induces fusion of the vector and the endosome membrane. Many previously-proposed VSV-G alternatives lack the required fusogenic action, which is fully preserved upon use of adapters of the present invention. Reversibility of the VSV-G-LDLR complex is greatly enhanced by a pH-dependent conformational change of the beta-propeller domain of LDLR. Unexpectedly, the present invention discloses that binding of VSV and VSV-G-pseudotyped vectors to Class A domains of LDLR is also reversible, despite lack of a beta-propeller structure, as these vectors are functional following endocytosis.

In one non-limiting specific embodiment of the present invention there is provided an adapter consisting essentially of the human proLDLR₍₂₅₋₁₄₅₎ (containing Class A repeats 1, 2 and 3, also referred to herein as sLDLR₂₅₋₁₄₅), fused at its C-terminus through a flexible polypeptide linker to the N-terminal domain of pro-carcinoembryonic antigen cell-adhesion molecule 8 [pro-CEACAM8₍₃₅₋₁₄₀₎]. Said Adapter, termed sLDLR-CEACAM8 is capable of forming a complex through its LDLR component with viruses and other vectors whose surfaces contain vesiculo-G Proteins. The resulting adapter-coated vectors (also referred to herein as adapter-modified viral particles) bind through the CEACAM8-derived component of the adapter to cells expressing CEACAM6 or CEACAM1, both serving as cell-surface binding proteins of CEACAM8 and subsequently internalized. One advantage of said adapter is the fact that it is made of human proteins and not expected to elicit an immune response. Without wishing to be bound by a specific theory or mechanism of action, said adapter may further mask immunogenic components such as VSV-G from neutralization by the immune system of the treated person.

In another non-limiting embodiment of the present invention there is provided an adapter consisting essentially of sLDLR₍₂₅₋₁₄₅₎ fused at its C-terminus through a flexible polypeptide linker to a monoclonal antibody or antibody fragment directed against CEACAM6. Said adapter, termed sLDLR-αCEACAM6 is capable of forming a complex through its LDLR component with viruses and other vectors whose surfaces exhibit any one or more of the vesiculo-G Proteins. These adapter-coated vectors bind through the αCEACAM6 component of the adapter to cells expressing CEACAM6 and subsequently internalized.

In another embodiment of the present invention there are provided complexes of sLDLR-CEACAM8 or sLDLR-αCEACAM6 with viruses decorated with vesiculo-G proteins. Said complexes may be used for selective viral oncolysis of tumor cells expressing high levels of CEACAM6 or CEACAM1, or their combinations. Example of said tumor cells are pancreatic tumor cells, known to express high levels of CEACAM6. In another embodiment of the present invention there are provided complexes of sLDLR-CEACAM8 or sLDLR-αCEACAM6 with recombinant forms of viruses decorated with vesiculo-G proteins and encoding sLDLR-CEACAM8 or sLDLR-αCEACAM6. Upon propagation of said recombinant viruses their progeny will bind sLDLR-CEACAM8 or sLDLR-αCEACAM6 directly upon budding from the virus-infected cells, thereby directing the progeny viruses to the tumor cells as well. In another embodiment of the present invention there are provided complexes of the adapter molecule sLDLR-CEACAM8 or sLDLR-αCEACAM6 with vectors pseudotyped with vesiculo-G proteins, and encoding the CFTR protein. These vectors may be used for transduction through the airway of lung epithelial cells, lung epithelial progenitor cells or other lung cells, known to express CEACAM6 and/or CEACAM1 at their apical side for gene therapy of cystic fibrosis.

In another specific non-limiting embodiment of the present invention there is provided a fusion protein consisting essentially of the human proLDLR₍₂₅₋₁₄₅₎ fused at its C-terminus through a flexible polypeptide linker to the human soluble c-KIT ligand, also termed SCF [KITLG(26-190)]. Said adapter, termed sLDLR-SCF, binds viruses and other vectors whose surfaces are decorated with vesiculo-G proteins, directing them by endocytosis into cells expressing the SCF receptor c-KIT. Said Adapter may be used for directing OV decorated with vesiculo-G proteins, as well as their recombinant forms encoding sLDLR-SCF, towards tumor cells expressing c-KIT. Examples of said tumor cells are breast tumor cells, small-cell lung cancer cells, gynecological tumors, and malignant glioma. Here too, one advantage of said adapter is the fact that it is made of human sequences and domains and therefore is not expected to elicit an immune response. Without wishing to be bound by a specific theory or mechanism of action, said adapter may further mask immunogenic components such as VSV-G from neutralization by the immune system of the treated person. In another specific non-limiting embodiment of the present invention there is provided a fusion protein consisting essentially of the human proLDLR₍₂₅₋₁₄₅₎ fused at its C-terminus through a flexible polypeptide linker to anti-c-KIT antibody or antibody domain. Said adapter, termed sLDLR-αKIT, binds viruses and other vectors decorated with vesiculo-G proteins, directing them by endocytosis into cells expressing c-KIT as demonstrated with other conjugates of anti c-KIT antibodies. In another embodiment of the present invention there are provided complexes of sLDLR-SCF with recombinant forms of viruses decorated with vesiculo-G proteins, and encoding sLDLR-SCF or sLDLR-αKIT. Said complexes may be used for viral oncolysis of tumor cells expressing c-KIT. Examples of said tumor cells are breast tumor cells, small-cell lung cancer cells, gynecological tumors, and malignant glioma. Another specific non-limiting embodiment of the present invention is directed to an adapter consisting essentially of human proLDLR₍₂₅₋₁₄₉₎, to which a ligand of PSMA is chemically coupled. Said adapter, termed sLDLR-PSMAL, binds viruses and other vectors whose surfaces are decorated with vesiculo-G proteins, directing them by endocytosis into cells expressing PSMA for the purpose of viral oncolysis. LDLR-PSMAL may be used to bind VSV or recombinant viruses decorated with vesiculo-G proteins, and encoding sLDLR-PSMAL for the purpose of viral oncolysis of prostate cancer cells. Example of cells expressing PSMA are prostate cancer cells.

Another specific non-limiting embodiment of the present invention is directed to an adapter consisting essentially of human proLDLR₍₂₅₋₁₄₅₎, fused at its C-terminus through a flexible polypeptide linker to a human monoclonal antibody or monoclonal antibody fragment directed against PSMA. Said Adapter, termed sLDLR-αPSMA, binds viruses and other vectors whose surfaces are decorated with vesiculo-G proteins, thereby directing them by endocytosis into cells expressing PSMA. sLDLR-αPSMA may be used to bind recombinant viruses decorated with vesiculo-G proteins, and encoding sLDLR-αPSMA for the purpose of viral oncolysis of prostate cancer cells. Designed Ankyrin Repeat Proteins (DARPins) are synthetic polypeptides showing a very high affinity towards a given target protein. A specific non-limiting embodiment of the present invention provides an adapter consisting essentially of human proLDLR₍₂₅₋₁₄₅₎ fused at its C-terminus through a flexible polypeptide linker to a DARPin selected for very high affinity towards PSMA. Said Adapter, termed sLDLR-PSMA-DARPin, may be used for directing VSV or recombinant viruses decorated with Vesiculo-G Proteins towards prostate cancer cells for the purpose of viral oncolysis. Another specific non-limiting embodiment of the present invention provides an adapter consisting essentially of human proLDLR₍₂₅₋₁₄₅₎ fused at its C-terminus through a flexible polypeptide linker to a DARPin selected for very high affinity towards human CD8. Said Adapter, termed sLDLR-53F6, may be used for transducing human cytotoxic T cells in vivo and in vitro by vectors encoding a chimeric antigen receptor (CAR). The resulting CAR-T cells may be used for treatment of various malignancies. A similar approach may be used for transducing natural killer (NK) cells or NK92 cells through CD56 to form CAR-NK cells in vivo and in vitro.

The following examples are presented in order to more fully illustrate some embodiments of the invention. They should, in no way be construed, however, as limiting the broad scope of the invention.

### Examples

### Example 1. Construction of pHLsec-sLDLR₍₂₅₋₁₈₇₎

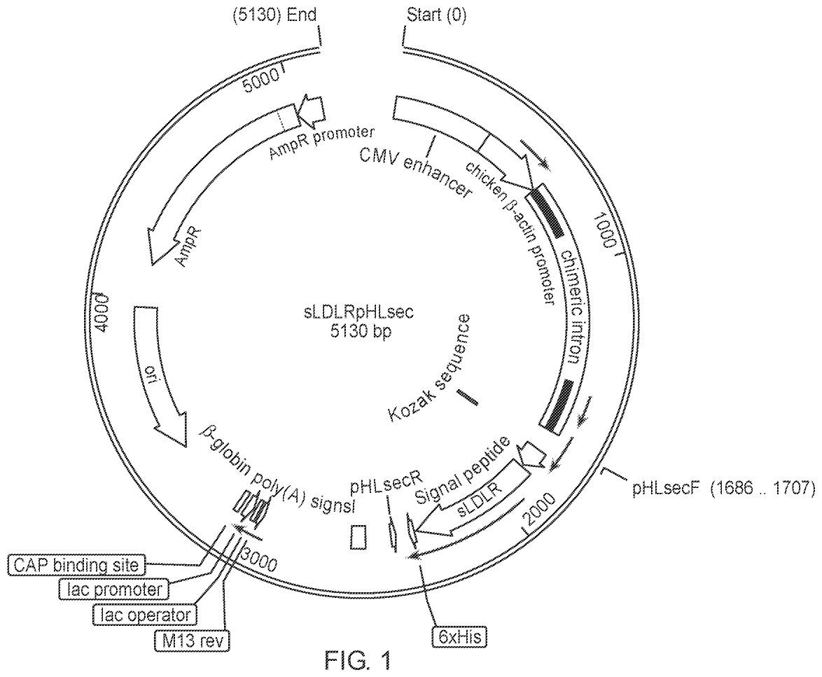

A nucleic acid construct encoding an isolated soluble form of low-density lipoprotein receptor (LDLR) was designed and manufactured. This construct contains a coding sequence for pro-human LDLR 25-187 (positions 25-187 of GenBank accession No. NP_000518.1), followed by a coding sequence for His Tag (GTKHHHHHH; SEQ ID NO: 20) in pHLsec vector. A schematic map of the resulting construct, herein designated “pHLsec-sLDLR₍₂₅₋₁₈₇₎” or sLDLRpHLsec, is provided in FIG. 1. The nucleic acid sequence of the pHLsec-sLDLR₂₅₋₁₈₇ is set forth in SEQ ID NO: 21. The amino acid sequences of the encoded human pro-LDLR₂₅₋₁₈₇ (containing cysteine-rich repeats 1-4, also referred to throughout the Examples and Figures as sLDLR₍₂₅₋₁₈₇₎ or sLDLR and the human pro-LDLR₍₂₅₋₁₈₇₎-His tag fusion protein, are set forth in SEQ ID NOs: 7 and 22, respectively, as follows:

(SLDLR₂₅₋₁₈₇, SEQ ID NO: 7)
DRCERNEFQCQDGKCISYKWVCDGSAECODGSDESQETCLSVTCKSGDFS
CGGRVNRCIPQFWRCDGQVDCDNGSDEQGCPPKTCSQDEFRCHDGKCISR
QFVCDSDRDCLDGSDEASCPVLTCGPASFQCNSSTCIPQLWACDNDPDCE
DGSDEWPQRCRGL.
(SLDLR₂₅₋₁₈₇-His-tag, SEQ ID NO: 22)
DRCERNEFQCQDGKCISYKWVCDGSAECQDGSDESQETCLSVTCKSGDFS
CGGRVNRCIPQFWRCDGQVDCDNGSDEQGCPPKTCSQDEFRCHDGKCISR
QFVCDSDRDCLDGSDEASCPVLTCGPASFQCNSSTCIPQLWACDNDPDCE
DGSDEWPQRCRGLGTKHHHHHH.

The corresponding nucleic acid sequences are set forth in SEQ ID NOs: 10 and 23, as follows:

(SLDLR₍₂₅₋₁₈₇₎ polynucleotide, SEQ ID NO: 10)
GACAGATGCGAAAGAAACGAGTTCCAGTGCCAAGACGGGAAATGCATCTC
CTACAAGTGGGTCTGCGATGGCAGCGCTGAGTGCCAGGATGGCTCTGATG
AGTCCCAGGAGACGTGCTTGTCTGTCACCTGCAAATCCGGGGACTTCAGC
TGTGGGGGCCGTGTCAACCGCTGCATTCCTCAGTTCTGGAGGTGCGATGG
CCAAGTGGACTGCGACAACGGCTCAGACGAGCAAGGCTGTCCCCCCAAGA
CGTGCTCCCAGGACGAGTTTCGCTGCCACGATGGGAAGTGCATCTCTCGG
CAGTTCGTCTGTGACTCAGACCGGGACTGCTTGGACGGCTCAGACGAGGC
CTCCTGCCCGGTGCTCACCTGTGGTCCCGCCAGCTTCCAGTGCAACAGCT
CCACCTGCATCCCCCAGCTGTGGGCCTGCGACAACGACCCCGACTGCGAA
GATGGCTCGGATGAGTGGCCGCAGCGCTGTAGGGGTCTTTAA,
and
(SLDLR₍₂₅₋₁₈₇₎-His-tag construct, SEQ ID NO: 23)
GACAGATGCGAAAGAAACGAGITCCAGTGCCAAGACGGGAAATGCATCTC
CTACAAGTGGGTCTGCGATGGCAGCGCTGAGTGCCAGGATGGCTCTGATG
AGTCCCAGGAGACGTGCTTGTCTGTCACCTGCAAATCCGGGGACTTCAGC
TGTGGGGGCCGTGTCAACCGCTGCATTCCTCAGTTCTGGAGGTGCGATGG
CCAAGTGGACTGCGACAACGGCTCAGACGAGCAAGGCTGTCCCCCCAAGA
CGTGCTCCCAGGACGAGTTTCGCTGCCACGATGGGAAGTGCATCTCTCGG
CAGTTCGTCTGTGACTCAGACCGGGACTGCTTGGACGGCTCAGACGAGGC
CTCCTGCCCGGTGCTCACCTGTGGTCCCGCCAGCTTCCAGTGCAACAGCT
CCACCTGCATCCCCCAGCTGTGGGCCTGCGACAACGACCCCGACTGCGAA
GATGGCTCGGATGAGGGTACCAAGCACCACCATCACCATCACTAA.

### Example 2. Production and Evaluation of sLDLR₍₂₅₋₁₈₇₎

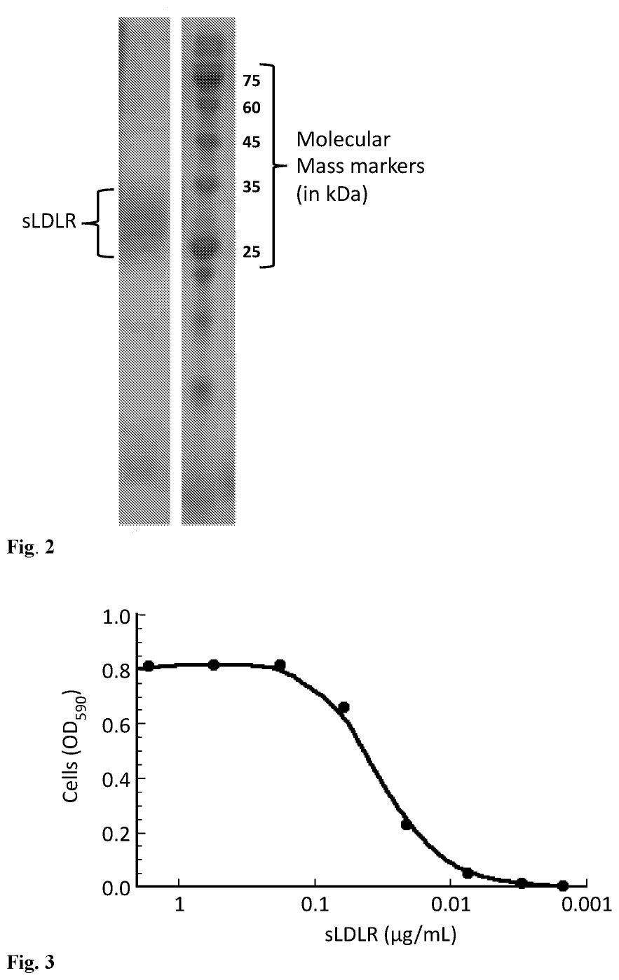

HEK293T cells were grown to 90% confluency in DMEM supplemented with non-essential amino acids (NEAA), Glutamine and 10% Fetal bovine serum, 18 mL per plate in 15×20 cm plates at 37° C. in 5% CO₂. The media was changed to DMEM supplemented with NEAA and Glutamine 14 mL. The cultures were transfected with pHLsec-sLDLR (20 μg DNA/plate) mixed with jetPEI (40 μL, Polyplus). Five h post transfection the media were changed to DMEM supplemented with NEAA and Glutamine, 20 mL/plate and the plates were further incubated for 67 h. Media (350 mL) were then collected, clarified by centrifugation (1000×g, 10 min at 4° C.) and dialyzed against a Binding buffer (HEPES 10 mM pH 7.4, NaCl 140 mM and CaCl2 0.2 g/L). The product was then captured using HisTrap HP column (5 mL, GE Healthcare) at 5 mL/min. The column was then washed with 25 mL Wash 1 buffer (Binding buffer plus 20 mM imidazole) followed by 20 mL with Wash 2 buffer (Binding buffer plus 50 mM imidazole). The product was then eluted with elution buffer: (Binding buffer plus 500 mM imidazole). The final volume was 10 mL. Calculated molecular weight: 19123.75. Calculated absorbance of 1 mg/mL solution at 280 nm is 1.307. Concentration of Batch #30284 is 0.25 mg/mL. As can be seen in FIG. 2, SDS-PAGE gave a single band of 25-35 kDa, having a broad appearance characteristic of glycoproteins.

For functional evaluation, HeLa cells (1.2×10⁵/mL, in 0.1 mL of DMEM supplemented with Penicillin 100 IU/mL, Streptomycin 0.1 mg/mL; sodium pyruvate 11 mg/mL and 10% Fetal bovine serum, hereinafter termed “DMEM-10”) were seeded in 96-well plates and grown to confluency (24 h at 37° C. in 5% CO₂). VSV was diluted separately in DMEM-10 to a concentration of 4×10⁶ pfu/mL, and aliquots of 0.1 mL in 96 well plates were pre-incubated for 15 min at room temperature with 3-fold serially diluted sLDLR₂₅₋₁₈₇, initial concentration 15 μg/mL. The VSV aliquots were then added to the confluent cultures of HeLa cells and the cultures were further incubated at 37° C./5% CO₂ for 18 h. The media were then discarded, the cells were fixed for 15 min with cold Methanol and then stained with 5% w/v crystal violet in 66% v/v methanol. The plates were then washed and the cell viability was measured using an ELISA 96 well plate reader at 590 nm. Control untreated HeLa cell cultures and cultures treated with VSV without sLDLR were used as standards of 100% and 0% protection, respectively. The results, presented in FIG. 3, show that sLDLR₂₅₋₁₈₇ was effective in neutralizing cytopathic effect of VSV on HeLa cells in a dose-dependent manner. In FIG. 3. “Cells (OD₅₉₀)” reflects the relative number of viable cells determined by staining with Crystal violet and “sLDLR (μg/ml)” represents the concentration of adapter molecules during the pre-incubation stage.

### Example 3. Construction and Production of a Mammalian Expression Vector Encoding sLDLR-CEACAM8

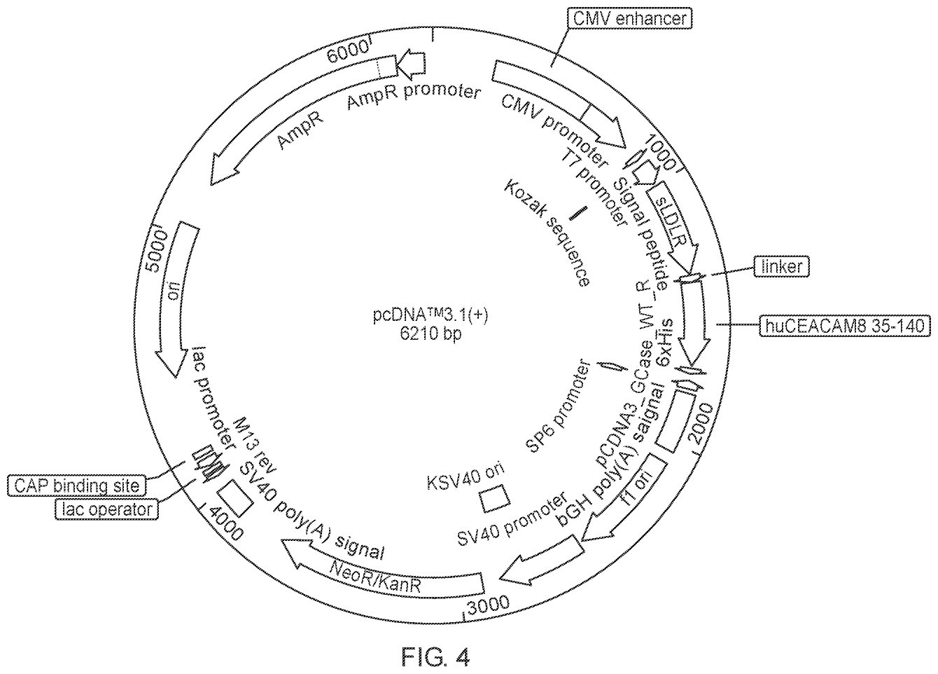

A nucleic acid construct encoding an isolated soluble form of LDLR fused to a targeting moiety derived from CEA Cell Adhesion Molecule 8 (CEACAM8), was designed and manufactured. This construct contains a coding sequence of human pro-LDLR₍₂₅₋₁₄₅₎ (positions 25-145 of GenBank accession No. NP_000518.1), a flexible linker (GSGGGGSGG, SEQ ID NO: 24), human pro-CEACAM8₍₃₅₋₁₄₀₎ (positions 35-140 of GenBank accession No. NP_001807.2) and His Tag (SEQ ID NO: 20) in pcDNA3.1 vector. A schematic map of the resulting construct, herein designated “pcDNA3-sLDLR-CEACAM8”, is provided in FIG. 4. The nucleic acid sequence of the pcDNA3-sLDLR-CEACAM8 construct is set forth in SEQ ID NO: 25.

The amino acid sequences of the encoded mature fusion protein, also referred to herein as sLDLR-CEACAM8, is set forth in SEQ ID NO: 17, as follows:

(SLDLR-CEACAM8, SEQ ID NO: 17)
DRCERNEFQCQDGKCISYKWVCDGSAECQDGSDESQETCLSVTCKSGDFS
CGGRVNRCIPQFWRCDGQVDCDNGSDEQGCPPKTCSQDEFRCHDGKCISR
QFVCDSDRDCLDGSDEASCPVGSGGGGSGGKEICGNPVTDNVKDITKLVA
NLPNDYMITLNYVAGMDVLPSHCWLRDMVIQLSLSLTILLDKFSNISEGL
SNYSIIDKLGKIVDDLVLCMEENAPKNIKESPKRPETRSFTPEEFFSIFN
RSIDAFKDFMVASDTSDCVLSSTLGPEKDSRVSVTKPFMLPPVAAGTKHH
HHHH.

The corresponding nucleic acid sequence, encoding for human pro-LDLR₍₂₅₋₁₄₅₎-Linker-human pro-CEACAM8₍₃₅₋₁₄₀₎-His tag and a stop codon, is set forth in SEQ ID NO: 26, as follows:

(sLDLR-CEACAM8-His tag construct, SEQ ID NO: 26)
GACAGATGCGAAAGAAACGAGTTCCAGTGCCAAGACGGGAAATGCATCTC
CTACAAGTGGGTCTGCGATGGCAGCGCTGAGTGCCAGGATGGCTCTGATG
AGTCCCAGGAGACGTGCTTGTCTGTCACCTGCAAATCCGGGGACTTCAGC
TGTGGGGGCCGTGTCAACCGCTGCATTCCTCAGTTCTGGAGGTGCGATGG
CCAAGTGGACTGCGACAACGGCTCAGACGAGCAAGGCTGTCCCCCCAAGA
CGTGCTCCCAGGACGAGTTTCGCTGCCACGATGGGAAGTGCATCTCTCGG
CAGTTCGTCTGTGACTCAGACCGGGACTGCTTGGACGGCTCAGACGAGGC
CTCCTGCCCGGTGGGCTCTGGAGGAGGIGGGAGCGGCGGACAGCTCACTA
TTGAAGCTGTGCCATCCAATGCTGCAGAGGGGAAGGAGGTTCTTCTACTT
GTCCACAATCTGCCCCAGGACCCTCGTGGCTACAACTGGTACAAAGGGGA
AACAGTGGATGCCAACCGTCGAATTATAGGATATGTAATATCAAATCAAC
AGATTACCCCAGGGCCTGCATACAGCAATCGAGAGACAATATACCCCAAT
GCATCCCTGCTGATGCGGAACGTCACCAGAAATGACACAGGATCCTACAC
CCTACAAGTCATAAAGCTAAATCTTATGAGTGAAGAAGTAACTGGCCAGT
TCAGCGTAGGTACCAAGCACCACCATCACCATCACTAA.

The corresponding nucleic acid sequence, encoding for human pro-LDLR₍₂₅₋₁₄₅₎-Linker-human pro-CEACAM8₍₃₅₋₁₄₀₎ and a stop codon, is set forth in SEQ ID NO: 27, as follows:

(sLDLR-CEACAM8-construct, SEQ ID NO: 27)
GACAGATGCGAAAGAAACGAGTTCCAGTGCCAAGACGGGAAATGCATCTCCTACAAGTGGGTCT
GCGATGGCAGCGCTGAGTGCCAGGATGGCTCTGATGAGTCCCAGGAGACGTGCTTGTCTGTCAC
CTGCAAATCCGGGGACTTCAGCTGTGGGGGCCGTGTCAACCGCTGCATTCCTCAGTTCTGGAGG
TGCGATGGCCAAGTGGACTGCGACAACGGCTCAGACGAGCAAGGCTGTCCCCCCAAGACGTGCT
CCCAGGACGAGTTTCGCTGCCACGATGGGAAGTGCATCTCTCGGCAGTTCGTCTGTGACTCAGA
CCGGGACTGCTTGGACGGCTCAGACGAGGCCTCCTGCCCGGTGGGCTCTGGAGGAGGTGGGAGC
GGCGGACAGCTCACTATTGAAGCTGTGCCATCCAATGCTGCAGAGGGGAAGGAGGTTCTTCTAC
TTGTCCACAATCTGCCCCAGGACCCTCGTGGCTACAACTGGTACAAAGGGGAAACAGTGGATGC
CAACCGTCGAATTATAGGATATGTAATATCAAATCAACAGATTACCCCAGGGCCTGCATACAGC
AATCGAGAGACAATATACCCCAATGCATCCCTGCTGATGCGGAACGTCACCAGAAATGACACAG
GATCCTACACCCTACAAGTCATAAAGCTAAATCTTATGAGTGAAGAAGTAACTGGCCAGTTCAG
CGTATAA.

The amino acid sequences of specific elements within this fusion protein, including human pro-LDLR₍₂₅₋₁₄₅₎; NP_000518.1; (containing cysteine-rich repeats 1-3, also referred to herein as sLDLR₍₂₅₋₁₄₅₎, SEQ ID NO: 5), and human pro-CEACAM8₍₃₅₋₁₄₀₎; NP_001807.2 (containing the first Ig-like domain; SEQ ID NO: 12), are as follows:

(sLDLR₍₂₅₋₁₄₅₎, SEQ ID NO: 5)
DRCERNEFQCQDGKCISYKWVCDGSAECQDGSDESQETCLSVTCKSGDFSCGGRVNRCIPQFWR
CDGQVDCDNGSDEQGCPPKTCSQDEFRCHDGKCISRQFVCDSDRDCLDGSDEASCPV;
(human pro-CEACAM8₍₃₅₋₁₄₀₎, SEQ ID NO: 12)
QLTIEAVPSNAAEGKEVLLLVHNLPQDPRGYNWYKGETVDANRRIIGYVISNQQITPGPAYSNR
ETIYPNASLLMRNVTRNDTGSYTLQVIKLNLMSEEVTGQFSV.

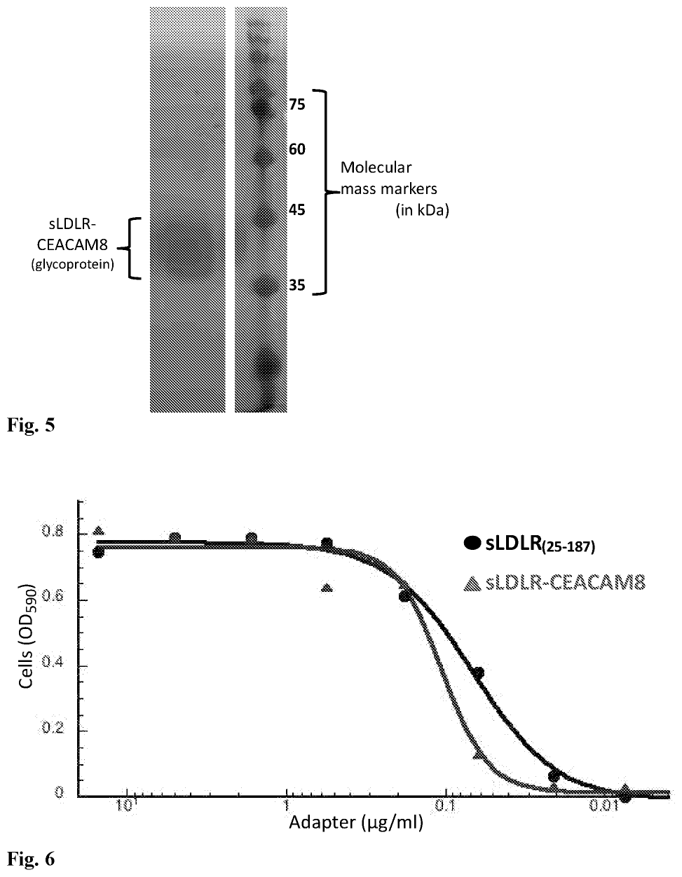

sLDLR-CEACAM8 was produced in HEK293T cells transfected with pcDNA3-sLDLR-CEACAM8 (SEQ ID NO: 25) essentially as described in Example 2. It was then purified essentially as described in Example 2. The final volume was 10 mL. Calculated molecular weight: 26,930.59. Ext. coefficient: 28055. Concentration: 0.3 mg/mL (11 μM) as determined by A280. As can be seen in FIG. 5. SDS-PAGE gave a single band of 35-45 kDa, having a broad appearance characteristic of glycoproteins.

### Example 4. Neutralization of VSV Cytopathic Effect in HeLa Cells with sLDLR-CEACAM8

HeLa cells (1.2×10⁵/mL, in 0.1 mL of DMEM-10) were seeded in 96-well plates and grown to confluency (24 h at 37° C. in 5% CO₂). VSV was diluted separately in DMEM-10 to a concentration of 4×10⁶ pfu/mL, and aliquots of 0.1 mL in 96 well plates were pre-incubated for 15 min at room temperature with 3-fold serially diluted sLDLR-CEACAM8 (initial concentration 15 μg/mL), or sLDLR₂₅₋₁₈₇ as a control. The VSV aliquots were then added to the confluent cultures of HeLa cells and the cultures were further incubated at 37° C./5% CO₂ for 18 h. The media were then discarded, the cells were fixed for 15 min with cold Methanol and then stained with 5% w/v crystal violet in 66% v/v methanol. The plates were then washed and the cell viability was measured using an ELISA 96 well plate reader at 590 nm. Control untreated HeLa cell cultures and cultures treated with VSV without adapters were used as standards of 100% and 0% protection, respectively.

The results are presented in FIG. 6, in which circles represent sLDLR₂₅₋₁₈₇, triangles represent sLDLR-CEACAM8, “Cells (OD₅₉₀)” reflects the relative number of viable cells determined by staining with Crystal violet and “Adapter (μg/ml)” represents the concentration of adapter molecules during the pre-incubation stage. As can be seen in FIG. 6, both sLDLR₂₅₋₁₈₇ and sLDLR-CEACAM8 were effective in neutralizing the cytopathic effect of VSV on HeLa cells, exhibiting dose-response curves.

### Example 5. Selective Lysis of Colon Cancer CaCo2 Cells by VSV in the Presence of sLDLR-CEACAM8

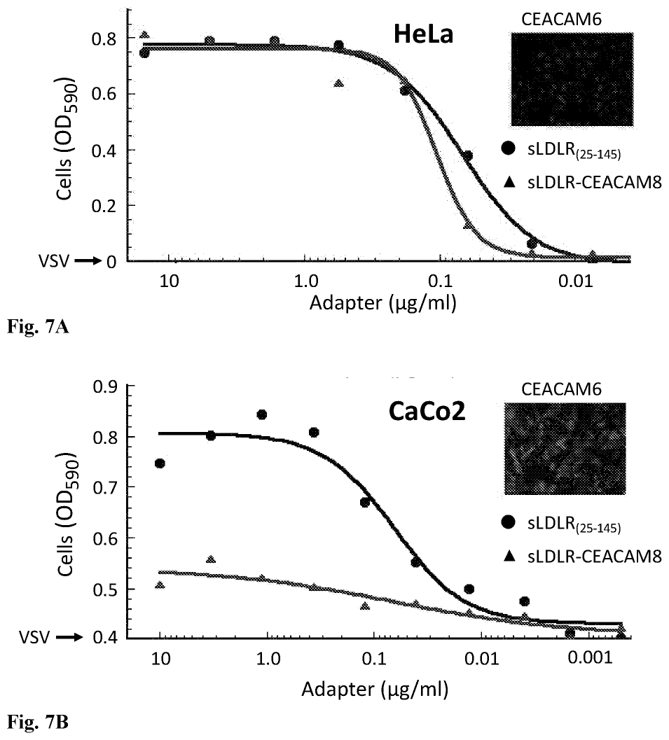

CaCo2 cells (CEACAM6-expressing colon cancer cells, 5×10⁵/mL, in 0.1 mL of DMEM supplemented with Penicillin 100 IU/mL, Streptomycin 0.1 mg/mL; sodium pyruvate 11 mg/mL, glutamine 2 mM, 1% non-essential amino acids, Merck Stock M7145, and 20% Fetal bovine serum, hereinafter termed “DMEM-20”) were seeded in 96-well plates and grown to confluency (24 h at 37° C. in 5% CO₂). VSV was diluted separately in DMEM-20 to a concentration of 2.5×10⁵ pfu/mL, and aliquots of 0.1 mL in 96 well plates were pre-incubated for 15 min at room temperature with 3-fold serially diluted sLDLR-CEACAM8 or sLDLR₂₅₋₁₈₇, initial concentration 10 μg/mL. The VSV aliquots were then added to the confluent cultures of CaCo2 cells and the cultures were further incubated at 37° C./5% CO₂ for 30 h. The media were then discarded, the cells were fixed, stained, washed and analyzed as described in Example 4. The results and are presented in FIGS. 7A (showing the effect on Hela cells as described in Example 4 and FIG. 6) and 7B (showing the effects in CaCo2 cells). Circles represent sLDLR₂₅₋₁₈₇ (depicted as “sLDLR”) and triangles represent sLDLR-CEACAM8. “VSV” represents addition of VSV only, without adapters.

HeLa and CaCO2 cells were also immuno-stained for the presence of CEACAM6 as follows: CaCo2 cells (100,000/well in DMEM-20 0.1 mL/well) and HeLa cells (24,000/well in DMEM-10 0.1 mL/well) were seeded in wells of ibidi 8 well slide and incubated at 37° C./5% CO₂ overnight. Next day, the cells were washed twice with PBS and fixed with paraformaldehyde (4% in water, 10 min. at room temperature), washed 3× with PBS and blocked with horse serum (10% in PBS, 45 min. at room temperature). Mouse anti-CEACM6 antibody (Santa Cruz, sc59899) was diluted 1:50 in bovine serum albumin (BSA, 3% in PBS 0.45 mL), added to the cells and incubated for 2 h at room temperature. Next, cells were washed with 3% BSA in PBS and incubated with secondary Cy3-donkey anti mouse antibody (1:200 in PBS containing 10% human serum (0.45 mL, 1.5 h at room temperature in the dark). The cells were then washed 3× with PBS and images were taken using the Nikon eclipse Ti fluorescence microscope with excitation at 540 nm and emission at 600 nm for Cy3. The results are shown as inserts (“CAECAM IHC”) in FIGS. 7A-7B (7A—HeLa cells, 7B—CaCo2 cells), in which CEACAM6 staining is shown (CEACAM6 IHC).

As can be seen in FIGS. 7A-7B, in CaCo2 cells, sLDLR₂₅₋₁₈₇ significantly neutralized the cytopathic effect of VSV, as in Hela cells. In contradistinction, the cytopathic effect of VSV on CaCo2 cells was substantially retained even in the presence of sLDLR-CEACAM8, whereas it was substantially neutralized in Hela cells. as can further be seen, CaCo2 cells stained positive for CEACAM6, a natural binding partner of CEACAM8, whereas Hela cells did not. Thus, sLDLR-CEACAM8 was capable of mediating VSV-induced oncolysis in CEACAM6-expressing colon cancer target cells, while inhibiting oncolysis in non-target Hela cells, demonstrating remarkable potency and target selectivity.

### Example 6. Selective Lysis of AsPC-1 Pancreatic Cancer Cells by VSV in the Presence of sLDLR-CEACAM8

AsPC-1 cells (CEACAM6-expressing pancreatic cancer cells. 1×10⁶/mL, in 0.1 mL of RPMI 1640 medium supplemented with Penicillin 100 IU/mL. Streptomycin 0.1 mg/mL; sodium pyruvate 11 mg/mL, glutamine 2 mM and 10% Fetal bovine serum, hereinafter termed “RPMI-10”) were seeded in 96-well plates and incubated for 2 h at 37° C. in 5% CO₂. VSV was diluted separately in RPMI-10 to a concentration of 15.000 pfu/mL, and aliquots of 0.1 mL in 96 well plates were pre-incubated for 15 min at room temperature with 2-fold serially diluted sLDLR-CEACAM8 or sLDLR₂₅₋₁₈₇, initial concentration 5 μg/mL. The VSV aliquots were then added to the confluent cultures of AsPC-1 cells and the cultures were further incubated at 37° C./5% CO₂ for 24 h. The media were then discarded, the cells were fixed, stained, washed and analyzed as described in Example 4. Control untreated AsPC-1 cell cultures and cultures treated with VSV without either sLDLR₍₂₅₋₁₈₇₎ or sLDLR-CEACAM8 were used as standards of 100% and 0% protection, respectively.

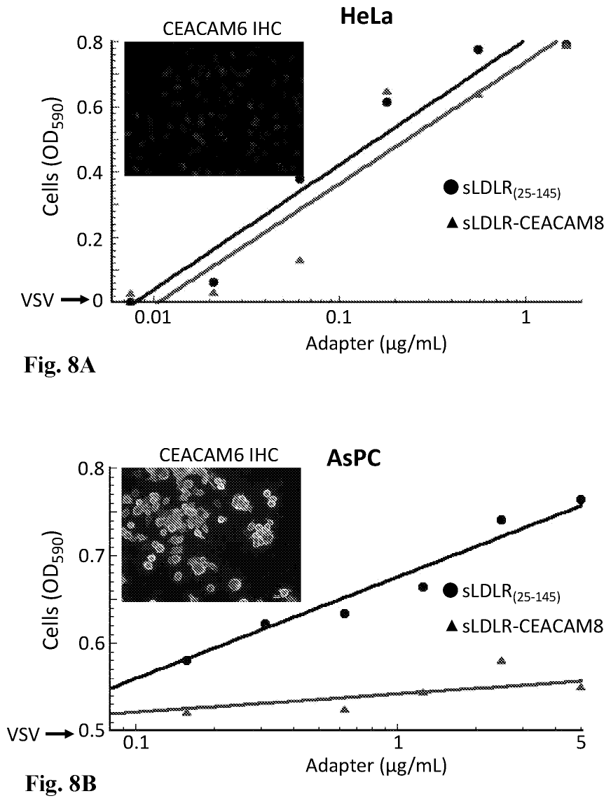

HeLa and AsPC-1 cells were also immuno-stained for the presence of CEACAM6 essentially as described in Example 5. The results are shown as inserts in FIG. 8A-8B (HeLa cells and AsPC-1 cells, respectively), in which CEACAM6 staining is shown (CEACAM6 IHC) and the labels are as indicate in FIG. 7. As can be seen in FIGS. 8A-8B, sLDLR-CEACAM8 was capable of mediating VSV-induced oncolysis in CEACAM6-expressing pancreatic cancer target cells, while inhibiting oncolysis in non-target HeLa cells, demonstrating remarkable potency and target selectivity.

### Example 7. SLDLR and sLDLR-CEACAM8 Effectively Inhibit Transduction of HeLa Cells by a Lentiviral Vector (LVV)

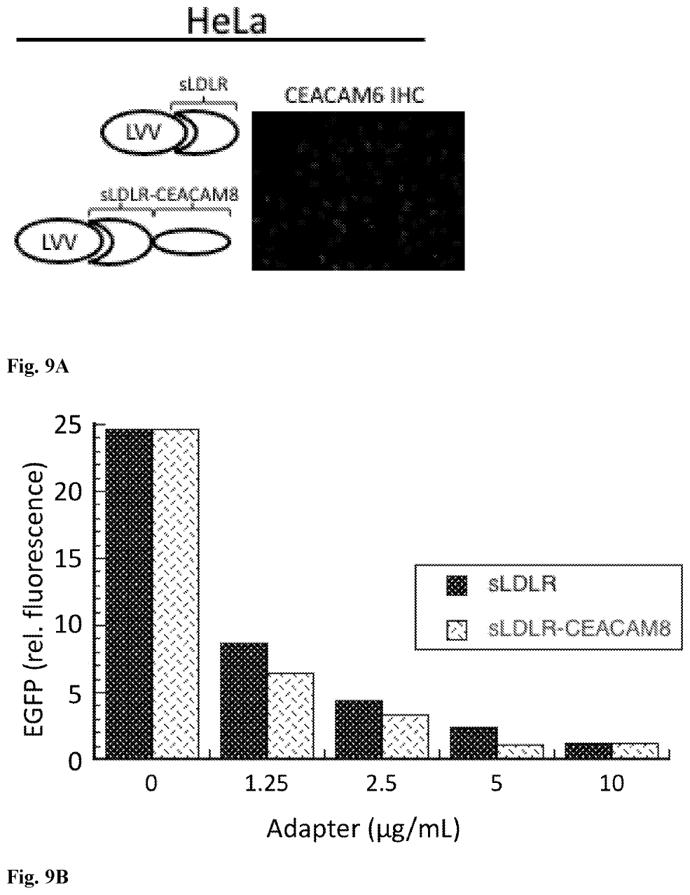

A LVV encoding enhanced green fluorescent protein (eGFP) was produced essentially as described by Lana and Strauss (in: Methods in Molecular Biology, 2020, vol. 2086, pp. 61-67). The resulting LVV stock was diluted 1:1 in Opti-MEM™ and aliquots of 60 μL/well were dispensed in a 96 well plate. Adapter proteins sLDLR-CEACAM8 and sLDLR (10 μg/mL) were two-fold serially diluted in lentivirus-containing Opti-MEM™ mentioned above in 96 well plate (60 μL/well). The resulting vector-adapter mixtures were incubated for 15 minutes at room temperature. HeLa cells (5×10⁵ cells/mL in DMEM-10) were seeded in 96-well plates (0.1 mL/well) and incubated at 37° C./5% CO₂ for 4 h. The culture medium of the HeLa cells in 96 well plate was removed and replaced by the vector-adapter mixture (30 μL/well). The cultures were incubated for 4 h in the presence of the mixture. DMEM-10 (0.17 mL/well) was then added, and the plates were-incubated for additional 24 h. All media was then replaced with fresh DMEM-10, 0.2 mL and the cells were then grown for additional 48 h. The cells were imaged for GFP by Nikon eclipse Ti fluorescence microscope 72 h post transduction using 470 nm excitation and 525 nm emission. The results are presented as relative fluorescence intensity in FIG. 9B, and immunohistochemistry staining for CEACAM6, along with a schematic representation of the adapters, is presented in FIG. 9A. The results show that in the absence of adapters, Hela cells transduced with the VSV-G-pseudotyped, eGFP-encoding LVV, showed significant fluorescent activity, and that both adapters significantly prevented the transduction.

### Example 8. Selective Transduction of CaCo2 Cells by LVV in the Presence of sLDLR-CEACAM8

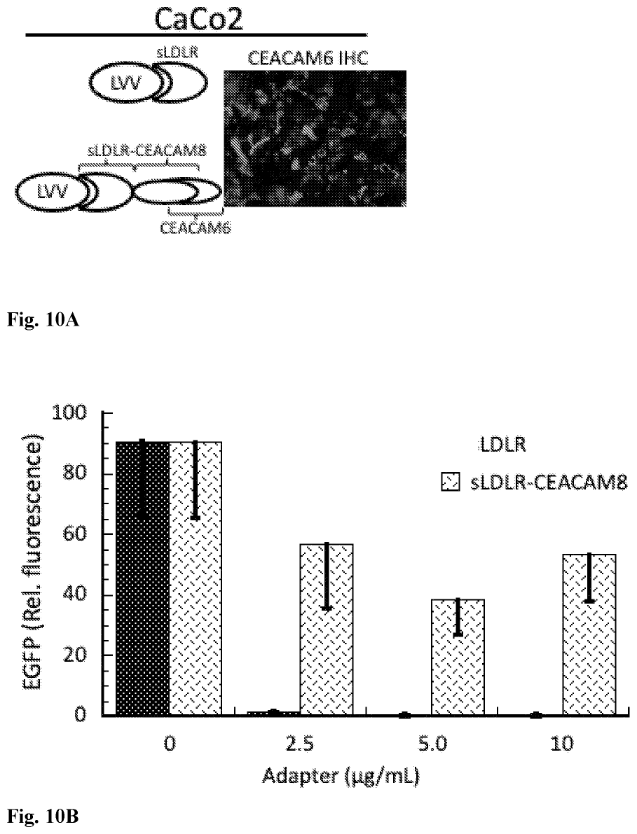

CaCO2 cells were seeded at the concentration of 1×10⁶ cells/mL, 0.1 ml, in 96-well plate for 4-hour attachment. Cells were grown in DMEM supplemented with 1% PenStrep, 1% NEAA, 1% L-glutamine and 20% Fetal bovine serum, at 37° C. in 5% CO₂. Next, Lentiviral particles were diluted 1:2 in Opti-MEM™ and pre-incubated with serially diluted sLDLR-CEACAM8 or sLDLR as a control for 15 minutes at room temperature. The adapters were 2-fold serially diluted starting from 10 μg/mL. Media were removed from cells and 30 μl of the LVV-adapter mix (as described in Example 7) was added to cells for 4 hours, then, 170 μl of media with serum was added to the cells for 24 hours. Media then was replaced, and cells were imaged using the Nikon eclipse Ti microscope 72 hours post transduction. The results are presented as relative fluorescence intensity in FIG. 10B, and immunohistochemistry staining for CEACAM6, along with a schematic representation of the adapters, is presented in FIG. 10A.

The results presented as relative fluorescence intensity in FIG. 10B show that sLDLR-CEACAM8 enabled the transduction of CEACAM6-expressing CaCo2 cells by the VSV-G-pseudotyped, eGFP-encoding LVV. In contradistinction, sLDLR prevented the transduction of CaCo2 cells completely, as in Hela Cells. Thus, the results demonstrate that sLDLR-CEACAM8 was capable of mediating selective transduction of VSV-G-pseudotyped LVVs to CEACAM6-expressing colon cancer cells, with remarkable potency and target selectivity, through CEACAM6 and not through LDLR.

In summary, described herein is the construction, production and use of a recombinant fusion protein, termed sLDLR-CEACAM8, capable of serving as an adapter directing viruses and viral vectors to specific target cells. Unexpectedly, the adapters were capable of facilitating effective and selective transduction, resulting in VSV-induced oncolysis tumor oncolysis or LVV-mediated gene delivery, despite the absence of certain structural elements and domains hitherto considered to be involved in ligand entry. In particular, the adapter molecules included as an anchoring component a plurality of Class A cysteine-rich repeat motifs (CR) derived from human LDLR (hLDLR CR), namely CR1, CR2 and CR3 sequences. It is noted, that additional hLDLR functional domains, such as other CR sequences (CR4 to CR7) constituting the receptor's ligand binding portion. In addition, the adapter molecules notably lacked the LDLR beta-propeller domain (amino acid residues 396-664 of human pro-LDLR), considered to mediate endosomal release of the ligand bound to the receptor, thereby facilitating viral infection.

In particular, LDL has been shown to dissociate from plasma membrane LDLR at pH 5.5; if the entire EGFP-domain or the beta-propeller together with the EGF-C domain is absent from the receptor, LDL release is reduced to 10% compared to 100% for wild-type LDLR, and the receptor is rapidly degraded after ligand binding (Davis et al., Nature, 326(23), 760-765, 1987). Surprisingly, despite the lack of the beta-propeller and interacting domain, viral particles complexed with the adapter exhibited highly efficient and selective infection, at least comparable to that of the corresponding non-complexed viral particles, with no apparent impairment of infection and receptor degradation.

### Example 9. Transduction of Patient Lungs with a CFTR-Encoding LVV

A VSV-G pseudotyped LVV encoding human cystic fibrosis transmembrane conductance regulator (CFTR, Genebank Accession No. NP_000483.3), is constructed and produced essentially as described in Marquez et al. (Genes, 2019. 10(3): p. 218). The produced stock of CFTR-encoding LVV is adjusted to a concentration of 10¹⁰ to 10¹² TU, and suspended in a solution of sLDRL-CEACAM8 (10 μg/mL, in 5 mL saline). The resulting suspension comprises particles of adapter-vector complexes, and adapter molecules in excess of said viral particles. For administration to the patients, the suspension is formulated such that the total concentration of adapter molecules following inhalation is 0.01-0.1 μM. The suspension is introduced as aerosol for intrabronchial and interalveolar administration through the patient's mouth. The patient is then inspected periodically for the impact of this treatment on their health status.

### Example 10. Construction and Production of a Mammalian Expression Vector Encoding sLDLR-SCF

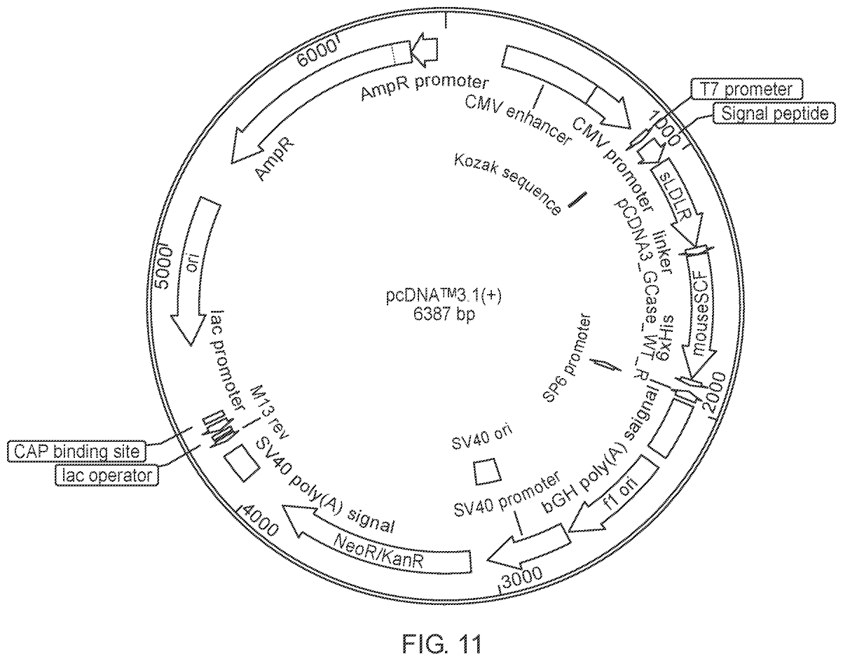

A nucleic acid construct encoding an isolated soluble form of LDLR fused to a targeting moiety derived from Stem cell factor (also known as SCF, KIT-ligand, KL, and steel factor) was designed and manufactured. This construct contains a coding sequence of mature human pro-LDLR₍₂₅₋₁₄₅₎ (positions 25-145 of GenBank accession No. NP_000518.1), a flexible linker (SEQ ID NO: 24), mouse pro-KITL₍₂₆₋₁₉₀₎ (positions 26-190 of GenBank accession No. NP_038626.1, also referred to as SCF) and His Tag (SEQ ID NO: 20) in pcDNA3.1 vector. A schematic map of the resulting construct, herein designated “pcDNA3-sLDLR-SCF”, is provided in FIG. 11, in which “sLDLR” indicates sLDLR₍₂₅₋₁₄₅₎ and “mouse SCF” indicates mouse pro-KITL₍₂₆₋₁₉₀₎. The nucleic acid sequence of the pcDNA3-sLDLR-SCF construct is set forth in SEQ ID NO: 28. The amino acid sequences of the encoded mature fusion protein, also referred to herein as sLDLR-SCF, is set forth in SEQ ID NO: 18, as follows:

(SLDLR-SCF, SEQ ID NO: 18)
DRCERNEFQCQDGKCISYKWVCDGSAECQDGSDESQETCLSVTCKSGDFSCGGRVNRCIPQFWR
CDGQVDCDNGSDEQGCPPKTCSQDEFRCHDGKCISRQFVCDSDRDCLDGSDEASCPVGSGGGGS
GGKEICGNPVTDNVKDITKLVANLPNDYMITLNYVAGMDVLPSHCWLRDMVIQLSLSLITLLDK
FSNISEGLSNYSIIDKLGKIVDDLVLCMEENAPKNIKESPKRPETRSFTPEEFFSIFNRSIDAF
KDFMVASDTSDCVLSSTLGPEKDSRVSVTKPFMLPPVAAGTKHHHHHH.

The nucleic acid sequence encoding human pro-LDLR₍₂₅₋₁₄₅₎-Linker-mouse pro-KITL₍₂₆₋₁₉₀₎-His tag and a stop codon is set forth in SEQ ID NO: 29, as follows:

(sLDLR-SCF construct, SEQ ID NO: 29)
GACAGATGCGAAAGAAACGAGTTCCAGTGCCAAGACGGGAAATGCATCTCCTACAAGTGGGTCT
GCGATGGCAGCGCTGAGTGCCAGGATGGCTCTGATGAGTCCCAGGAGACGTGCTTGTCTGTCAC
CTGCAAATCCGGGGACTTCAGCTGTGGGGGCCGTGTCAACCGCTGCATTCCTCAGTTCTGGAGG
TGCGATGGCCAAGTGGACTGCGACAACGGCTCAGACGAGCAAGGCTGTCCCCCCAAGACGTGCT
CCCAGGACGAGITTCGCTGCCACGATGGGAAGTGCATCTCTCGGCAGTTCGTCTGTGACTCAGA
CCGGGACTGCTTGGACGGCTCAGACGAGGCCTCCTGCCCGGTGGGCTCTGGAGGAGGTGGGAGC
GGCGGAAAGGAGATCTGCGGGAATCCTGTGACTGATAATGTAAAAGACATTACAAAACTGGTGG
CAAATCTTCCAAATGACTATATGATAACCCTCAACTATGTCGCCGGGATGGATGTTTTGCCTAG
TCATTGTTGGCTACGAGATATGGTAATACAATTATCACTCAGCTTGACTACTCTTCTGGACAAG
TTCTCAAATATTTCTGAAGGCTTGAGTAATTACTCCATCATAGACAAACTIGGGAAAATAGTGG
ATGACCTCGTGTTATGCATGGAAGAAAACGCACCGAAGAATATAAAAGAATCTCCGAAGAGGCC
AGAAACTAGATCCTTTACTCCTGAAGAATTCTTTAGTATTTTCAATAGATCCATTGATGCCTTT
AAGGACTTTATGGTGGCATCTGACACTAGTGACTGTGTGCTCTCTTCAACATTAGGTCCCGAGA
AAGATTCCAGAGTCAGTGTCACAAAACCATTTATGTTACCCCCTGTTGCAGCCGGTACCAAGCA
CCACCATCACCATCACTAA.

The amino acid sequences of human pro-LDLR₍₂₅₋₁₄₅₎, also referred to herein as sLDLR₂₅₋₁₄₅, SEQ ID NO: 8) is described in Example 3 above, and that of mouse pro-KITL(26-190) (also termed SCF or mSCF) is set forth in SEQ ID NO: 13 below:

(mSCF, SEQ ID NO: 13)
KEICGNPVTDNVKDITKLVANLPNDYMITLNYVAGMDVLPSHCWLRDMVIQLSLSLITLLDKFS
NISEGLSNYSIIDKLGKIVDDLVLCMEENAPKNIKESPKRPETRSFTPEEFFSIFNRSIDAFKD
FMVASDTSDCVLSSTLGPEKDSRVSVTKPFMLPPVAA.

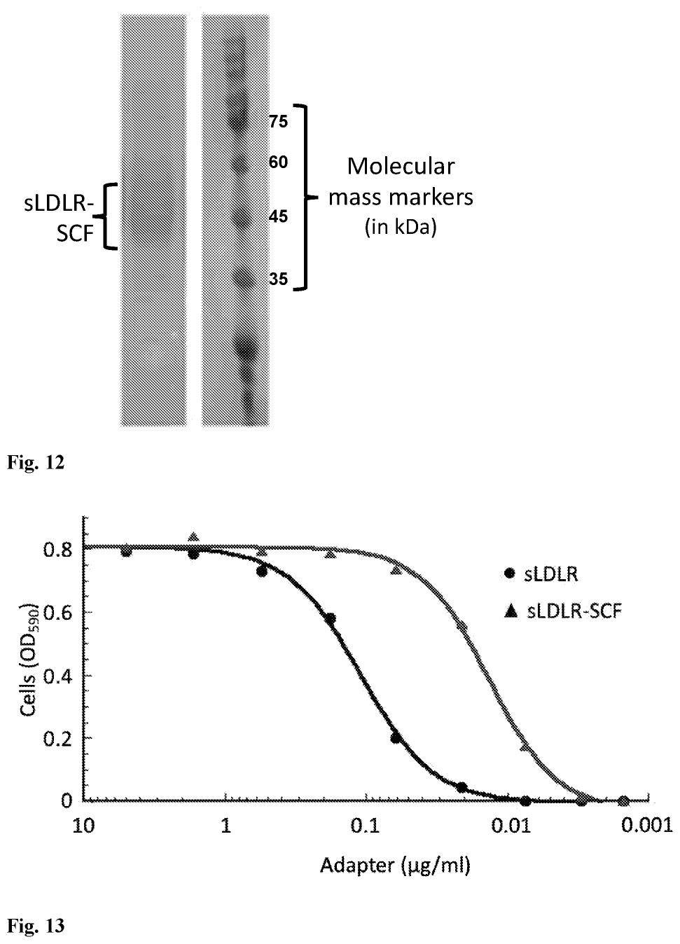

sLDLR-SCF was produced in HEK293T cells transfected with pcDNA3-sLDLR-SCF essentially as described in Example 2. It was then purified essentially as described in Example 2. It was then purified essentially as described in Example 2. The final volume was 10 mL. Calculated molecular weight: 26,930.59. Ext. coefficient: 28055. Concentration: 0.3 mg/mL (11 μM) as determined by A280. As can be seen in FIG. 12, SDS-PAGE gave a single band of molecular mass 45-55 kDa, having a broad appearance characteristic of glycoproteins.

A nucleic acid construct encoding an isolated soluble form of LDLR (sLDLR₂₅₋₁₄₅. SEQ ID NO: 5) fused to a targeting moiety derived from human SCF (human pro-KITL₍₂₆₉₋₇₆₃₎, also termed hSCF) is similarly produced. The amino acid sequence of hSCF is set forth in SEQ ID NO: 14:

(hSCF, SEQ ID NO: 14)
EGICRNRVTNNVKDVTKLVANLPKDYMITLKYVPGMDVLPSHCWISEMVVQLSDSLTDLLDKFS
NISEGLSNYSIIDKLVNIVDDLVECVKENSSKDLKKSFKSPEPRLFTPEEFFRIFNRSIDAFKD
FVVASETSDCVVSSTLSPEKDSRVSVTKPFMLPPVAA.

The amino acid sequence of human pro-LDLR₍₂₅₋₁₄₅₎-Linker-human pro-KITL₍₂₆₉₋₇₆₃₎-His tag and a stop codon is set forth in SEQ ID NO: 19, as follows:

(sLDLR-hSCF, SEQ ID NO: 19)
DRCERNEFQCQDGKCISYKWVCDGSAECQDGSDESQETCLSVTCKSGDFSCGGRVNRCIPQFWR
CDGQVDCDNGSDEQGCPPKTCSQDEFRCHDGKCISRQFVCDSDRDCLDGSDEASCPVGSGGGGS
GGEGICRNRVTNNVKDVTKLVANLPKDYMITLKYVPGMDVLPSHCWISEMVVQLSDSLIDLLDK
FSNISEGLSNYSIIDKLVNIVDDLVECVKENSSKDLKKSFKSPEPRLFTPEEFFRIFNRSIDAF
KDFVVASETSDCVVSSTLSPEKDSRVSVTKPFMLPPVAAGTKHHHHHH.

The nucleic acid sequence encoding human pro-LDLR₍₂₅₋₁₄₅₎-Linker-human pro-KITL₍₂₆₉₋₇₆₃₎-His tag and a stop codon is set forth in SEQ ID NO: 33, as follows:

(sLDLR-hSCF construct, SEQ ID NO: 33)
GACAGATGCGAAAGAAACGAGTTCCAGTGCCAAGACGGGAAATGCATCTCCTACAAGTGGGTCT
GCGATGGCAGCGCTGAGTGCCAGGATGGCTCTGATGAGTCCCAGGAGACGTGCTTGTCTGTCAC
CTGCAAATCCGGGGACTTCAGCTGTGGGGGCCGTGTCAACCGCTGCATTCCTCAGTTCTGGAGG
TGCGATGGCCAAGTGGACTGCGACAACGGCTCAGACGAGCAAGGCTGTCCCCCCAAGACGTGCT
CCCAGGACGAGTTTCGCTGCCACGATGGGAAGTGCATCTCTCGGCAGTTCGTCTGTGACTCAGA
CCGGGACTGCTTGGACGGCTCAGACGAGGCCTCCTGCCCGGTGGGCTCTGGAGGAGGTGGGAGC
GGCGGAGAAGGGATCTGCAGGAATCGTGTGACTAATAATGTAAAAGACGTCACTAAATTGGTGG
CAAATCTTCCAAAAGACTACATGATAACCCTCAAATATGTCCCCGGGATGGATGTTTTGCCAAG
TCATTGTTGGATAAGCGAGAIGGTAGTACAATTGTCAGACAGCTTGACTGATCTTCTGGACAAG
TTTTCAAATATTTCTGAAGGCTTGAGTAATTATTCCATCATAGACAAACTTGTGAATATAGTGG
ATGACCTTGTGGAGTGCGTGAAAGAAAACTCATCTAAGGATCTAAAAAAATCATTCAAGAGCCC
AGAACCCAGGCTCTTTACTCCTGAAGAATTCTTTAGAATTTTTAATAGATCCATTGATGCCTTC
AAGGACTTTGTAGTGGCATCTGAAACTAGTGATTGTGTGGTTTCTTCAACATTAAGTCCTGAGA
AAGATTCCAGAGTCAGTGTCACAAAACCATTIATGTTACCCCCTGTTGCAGCCGGTACCAAGCA
CCACCATCACCATCACTAA.

### Example 11. Neutralization of VSV Cytopathic Effect in HeLa Cells with sLDLR-SCF

HeLa cells (1.2×10⁵/mL, DMEM-10) were seeded in 96-well plates and grown to confluency (24 h at 37° C. in 5% CO₂). VSV was diluted separately in DMEM-10 to a concentration of 4×10⁶ pfu/mL, and pre-incubated with 3-fold serially diluted sLDLR-SCF (SEQ ID NO: 18) or sLDLR, initial concentration 15 μg/mL as described in Example 4. The VSV aliquots were then added to the confluent cultures of Hela cells and the cultures were further incubated at 37° C./5% CO₂ for 18 h. The media were then discarded, the cells were fixed, stained, washed and analyzed as described in Example 4. The results are presented in FIG. 13, in which “cells (OD) reflects the relative number of viable cells determined by staining with Crystal violet and “Adapter (μg/ml)” represents the concentration of adapter molecules during the pre-incubation stage. As can be seen in FIG. 13, both adapters were effective in neutralizing the cytopathic effect of VSV on HeLa cells, in a dose-dependent manner. As can further be seen, the effect of the two adapters was similar at high adapter concentrations (1-10 μg/ml), whereas at lower adapter concentrations, sLDLR-SCF exhibited significantly improved efficacy. The results indicate that sLDLR-SCF may be used at lower concentrations, and thus may improve safety without impairing therapeutic efficacy.

### Example 12. Preparation of sLDLR-PSMAL

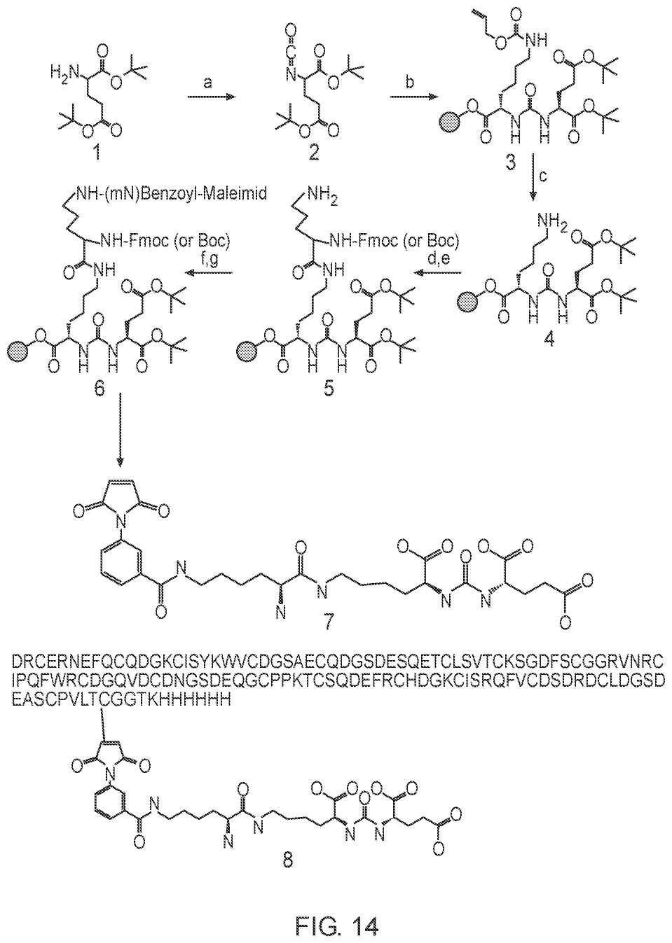

A conjugate of an isolated soluble form of LDLR (pro-LDLR 25-149, positions 25-149 of GenBank accession No. NP_000518.1, SEQ ID NO: 6) chemically linked to a targeting moiety derived from a prostate-specific membrane antigen ligand (PSMAL), was designed and produced. Human pro-LDLR 25-149, also referred to herein as sLDLR₍₂₅₋₁₄₉₎, contains, in addition to the sLDLR₍₂₅₋₁₄₅₎ anchoring region discussed above, a C′ LTCG linker corresponding to residues 146-149 of Human pro-LDLR, in which the cysteine residue is used in chemical conjugation of the targeting region. Chemical conjugation is performed essentially as described in Eder, M., et al., 2012 (Bioconjugate Chemistry, 23 (4): p. 688-697). The conjugation process is described in further detail below and illustrated in FIG. 14. The PSMAL comprises the human PSMA-specific Glu-NH—CO—NH-Lys pharmacophore described in Eder, et al., 2012 (ibid).

Briefly, In a first step (a), the isocyanate 2 of the glutamyl moiety is generated in situ by adding a mixture of 3 mmol of bis(tert butyl)-L-glutamate hydrochloride (Bachem, Switzerland) (1) and 1.5 mL of N-ethyldiisopropylamine (DIPEA) in 200 mL of dry CH₂Cl₂ to a solution of 1 mmol triphosgene in 10 mL of dry CH₂Cl₂ at 0° C. for 4 h. After agitation of the reaction mixture for one further hour at 25° C., 0.5 mmol of a resin-immobilized (2-chloro-trityl resin, Merck, Darmstadt) ε-allyloxycarbonyl protected lysine is added (b) in one portion (in 4 mL CH₂Cl₂) and reacted for 16 h with gentle agitation leading to compound 3. The resin is filtered off, washed with dry CH₂Cl₂ and the allyloxy protecting group is removed (c) using 100 mg tetrakis-(triphenyl) palladium (Sigma-Aldrich, Germany) and 400 μL morpholine in 4 mL CH₂Cl₂ for 3 h, resulting in compound 4. The resin is filtered off, washed with dry CH₂Cl₂ and the following coupling (d) of 2 mmol of 2-Fmoc-6-Alloc-Lys (Fmoc-Lys (Alloc)-OH), is performed using 1.96 mmol of HBTU (Merck, Darmstadt, Germany), and 2 mmol of N-ethyldiisopropylamine in a final volume of 4 mL dry CH₂Cl₂. The suspension is reacted for 16 h with gentle agitation. The resin is filtered off, washed with dry CH₂Cl₂ and the allyloxy protecting group is removed (c) using 100 mg tetrakis-(triphenyl) palladium (Merck, Germany) and 400 μL morpholine in 4 mL CH₂Cl₂ for 3 h to form 5. The following coupling of 2 mmol of maleimidobenzoyl-N-hydroxyssucinimide ester (MBS, Pierce) is performed (f) using 2 mmol of N-ethyl-diisopropylamine in a final volume of 4 mL dry CH₂Cl₂. The suspension is reacted for 16 h with gentle agitation to form 6. The resin is filtered off, washed with dry CH₂Cl₂ and the Fmoc group is removed (g, not needed if Boc is used instead of Fmoc) with 4 mL of 30% piperidine in DMF for 30 min with gentle agitation. The resin is filtered off, washed with dry CH₂Cl₂ and the resulting product is cleaved off the resin using 4 mL TFA (10% in H₂O) 1 h at room temperature. The product 7 (comprising the PSMA-specific pharmacophore and a flexible hydrocarbon chemical linker) is evaporated, dissolved in water and purified by preparative RP-HPLC. Product 7 (1 mg in 1 mL H₂O) is added to a solution of sLDLR₍₂₅₋₁₄₉₎ (1 mg in 50 mM aq. HEPES, pH 7.0, 1 mL). The mixture is kept overnight at room temperature and the resulting sLDLR-PSMAL 8 is isolated by size exclusion chromatography.

### Example 13. Construction of Recombinant VSV Encoding sLDLR-CEACAM8

A recombinant VSV encoding sLDLR-CEACAM8 is constructed essentially as follows: dsDNA was amplified using vector pcDNA3-sLDLR-CEACAM8 (SEQ ID NO: 25, described in Example 3) and the following primers (SEQ ID NOs: 30-31, respectively):

Forward:
5′-GGCTCGAGAAGCTAGCGCCACCATGGGGATCCT;
Reverse:
5′-CGCTCTAGATTATACGCTGAACTGGCCAGTTTACTTCTT.

The resulting sequence includes or encodes the following elements: an XhoI site, a Kozak sequence, a start codon, a signal peptide sequence, a pro-LDLR₍₂₅₋₁₄₅₎ a linker sequence, a pro-CEACAM8₍₃₅₋₁₄₀₎ sequence, a stop codon, and an XbaI site. The PCR product was digested with XhoI and XbaI and ligated into pVSV-XN1 (Addgene) that was pre-digested with XhoI and NheI. The resulting vector was named pVSV-XN1-sLDLR-CEACAM8. A recombinant VSV encoding sLDLR-CEACAM8 was prepared using pVSV-FL+(2) VSV Plasmid Expression Vector and helper plasmids set (VSV-n, VSV-P and VSV-L), all from Kerafast.com, and pVSV-XN1-sLDLR-CEACAM8. The resulting recombinant VSV vector encodes for an adapter protein comprising a sLDLR VSV-G-specific moiety and a cell targeting moiety derived from human CEACAM8. Accordingly, without wishing to be bound by a specific theory or mechanism of action, progeny of this vector produces the adapter in the host cells, forming VSV-adapter complexes.

### Example 14. Construction and Production of a Mammalian Expression Vector Encoding a CD8-Specific Adapter (sLDLR-53F6)

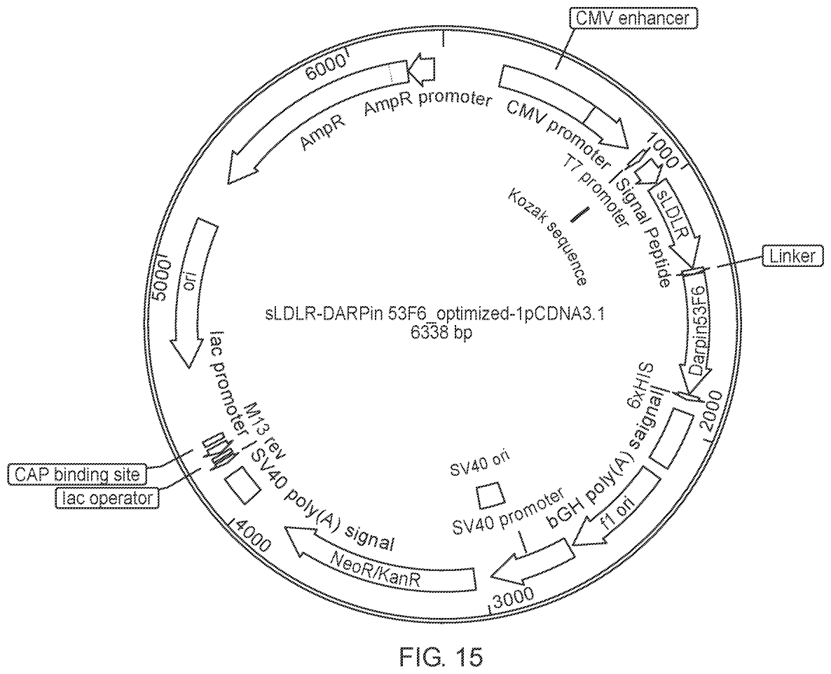

A nucleic acid construct encoding an isolated soluble form of LDLR fused to a targeting moiety derived from a designed ankyrin repeat protein (DARPin) specific to human CD8, was designed and manufactured. This construct contains a coding sequence of mature human pro-LDLR₍₂₅₋₁₄₅₎ (positions 25-145 of GenBank accession No. NP_000518.1), a flexible linker (GSGGGGS, SEQ ID NO: 34), DARPin 53F6 (described in Frank, A. M., et al., Human Gene Therapy, 2020. 31(11-12): p. 679-691, herein termed 53F6) and the His Tag in pcDNA3.1 vector. A schematic map of the resulting construct, herein designated pcDNA3-sLDLR-53F6, is provided in FIG. 15. The nucleic acid sequence of the pcDNA3-sLDLR-53F6 construct is set forth in SEQ ID NO: 35.

The amino acid sequences of the encoded mature fusion protein, also referred to herein as “sLDLR-DARPin53F6”, is set forth in SEQ ID NO: 16, as follows:

(SLDLR-DARPin53F6, SEQ ID NO: 16)
DRCERNEFQCQDGKCISYKWVCDGSAECQDGSDESQETCLSVTCKSGDFSCGGRVNRCIPQFWR
CDGQVDCDNGSDEQGCPPKTCSQDEFRCHDGKCISRQFVCDSDRDCLDGSDEASCPVGSGGGGS
DLGKKLLEASRAGQDDEVRILMANGADVNAQDRYGTTPLHLAAWHGHLEIVEVLLKHGADVNAN
DVKGNTPLHLAANVGHLEIVEVLLKYGADVNAADNWGFTPLHLAAFWGHLEIVEVLLKYGADVN
AQDKFGKTPFDLAIDNGNEDIAEVLQKAAGTKHHHHHH.

The corresponding nucleic acid sequence encoding the mature human pro-LDLR₍₂₅₋₁₄₅₎-Linker-DARPin 53F6-His tag and a stop codon is set forth in SEQ ID NO: 36, as follows:

(SLDLR-DARPin53F6 construct, SEQ ID NO: 36)
GATCGGTGCGAGAGAAACGAGTTCCAGTGCCAGGACGGCAAGTGTATCTCCTACAAGTGGGTGT
GCGATGGCTCTGCCGAGTGTCAGGATGGCAGCGACGAGTCCCAGGAGACATGCCTGTCTGTGAC
CTGTAAGTCTGGCGACTTCAGCTGCGGAGGAAGGGTGAACAGGTGTATCCCACAGTTTTGGCGG
TGCGATGGCCAGGTGGACTGTGATAATGGAAGCGACGAGCAGGGATGCCCACCIAAGACCTGTT
CCCAGGATGAGTTCCGGTGCCACGACGGCAAGTGTATCTCTCGGCAGTTCGTGTGCGACAGCGA
TAGAGACTGTCTGGATGGCTCCGACGAGGCCTCTTGTCCTGTGGGAAGCGGAGGAGGAGGATCC
GACCTGGGCAAGAAGCTGCTGGAGGCCTCCAGGGCAGGACAGGACGATGAAGTGAGGATCCTGA
TGGCCAACGGCGCCGATGTGAATGCCCAGGACAGATATGGAACCACACCACTGCACCTGGCAGC
ATGGCACGGACACCTGGAGATCGTGGAGGTGCTGCTGAAGCACGGCGCCGATGTGAACGCCAAT
GACGTGAAGGGAAACACCCCTCTGCATCTGGCAGCAAATGTGGGCCACCTGGAGATTGTCGAAG
TGCTGCTGAAGTATGGAGCCGATGTGAACGCCGCCGACAATTGGGGCTTCACACCTCTGCACCT
GGCCGCCTTTTGGGGCCACCTGGAGATCGTCGAAGTCCTGCTGAAATATGGAGCTGATGTGAAC
GCTCAGGACAAGTTCGGCAAGACCCCATTTGATCTGGCCATCGACAACGGCAATGAAGATATTG
CTGAAGTCCTGCAGAAGGCTGCTGGCACAAAACACCATCATCATCACCACTAA.

The amino acid sequences of human pro-LDLR₍₂₅₋₁₄₅₎ (SEQ ID NO: 5) is provided in Example 3 above, and that of DARPin53F6 is set forth in SEQ ID NO: 11, as follows:

(DARPin53F6, SEQ ID NO: 11)
DLGKKLLEASRAGQDDEVRILMANGADVNAQDRYGTTPLHLAAWHGHLEIVEVLLKHGADVNAN
DVKGNTPLHLAANVGHLEIVEVLLKYGADVNAADNWGFTPLHLAAFWGHLEIVEVLLKYGADVN
AQDKFGKTPFDLAIDNGNEDIAEVLQKAA.

sLDLR-53F6 was produced in HEK293T cells transfected with pcDNA3-sLDLR-53F6 essentially as described in Example 2. It was then purified essentially as described in Example 2. It was then purified essentially as described in Example 2. The final volume was 10 mL. Calculated molecular weight: 31,925.22. Ext. coefficient: 34,585. As can be seen in FIG. 16, SDS-PAGE gave a single band of apparent molecular mass of 33 kDa.

### Example 15. Neutralization of VSV Cytopathic Effect in Hela Cells with sLDLR-53F6

HeLa cells (1.2×10⁵/mL, in 0.1 mL of DMEM-10) were seeded in 96-well plates and allowed to attach for 2 h (at 37° C. in 5% CO₂). VSV was diluted separately in DMEM-10 to a concentration of 1×10⁶ pfu/mL, and aliquots of 0.1 mL in 96 well plates were pre-incubated for 15 min at room temperature with 2-fold serially diluted sLDLR-DARPin53F6, or sLDLR₍₂₅₋₁₈₇₎ as a control (initial concentrations 10 μg/mL) The VSV aliquots were then added to the confluent cultures of HeLa cells and the cultures were further incubated at 37° C./5% CO₂ for 18 h. The media were then discarded, the cells were then washed, fixed, stained and analyzed as described in Example 4.

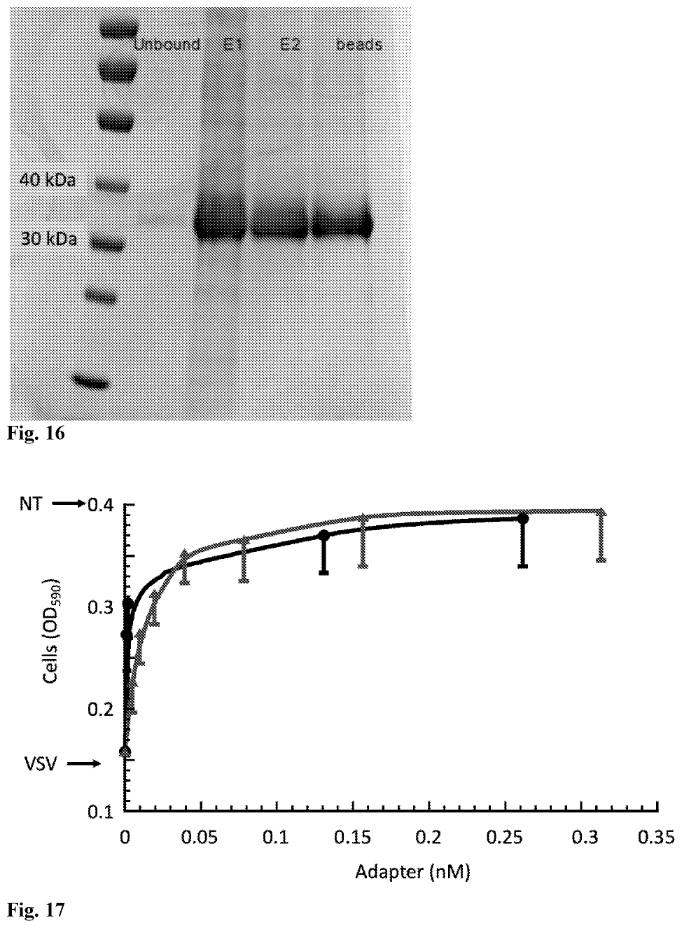

The results are presented in FIG. 17, in which “Cells (OD₅₉₀)” reflects the relative number of viable cells determined by staining with Crystal violet and “Adapter (nM)” represents the concentration of adapter molecules during the pre-incubation stage. OD₅₉₀ of untreated cells (NT) or cells treated with VSV without adapters (VSV) is indicated by arrows on the Y axis, and the equivalent nM concentrations are shown. Circles represent sLDLR₍₂₅₋₁₈₇₎ and triangles represent sLDLR-53F6. As can be seen in FIG. 17, both sLDLR₂₅₋₁₈₇ and sLDLR-DARPin53F6 were equally effective in neutralizing the cytopathic effect of VSV on HeLa cells, exhibiting dose-response curves. Hence, sLDLR-DARPin53F6 is an effective blocker of VSV-G.

### Example 16. In Vitro Transduction of CD8⁺ Molt4 Cells and PBMC Using sLDLR-53F6

eGFP-encoding lentiviral particles (LVV, pLVX-ZsGreen1-C1 vector, TaKaRa, Cat. #632565) 10⁶ TU/ml) are diluted 1:2 in Opti-MEM™ and pre-incubated for 15 min at room temperature with serially twofold diluted sLDLR or sLDLR-53F6 (starting at 10 μg/mL). Molt4.8 cells are seeded at the concentration of 1×10⁶ cells/mL, in 70 μl Opti-MEM™, in 96-well plate. Aliquots (30 μl) of the LVV with sLDLR or sLDLR-53F6 are added to the cells and the plate is incubated for 4 h at 37° C. and 5% CO₂. Then, 100 μl of DMEM supplemented with 20% fetal bovine serum is added to the cells and incubation continues for 24 h. Media is then replaced with DMEM supplemented with 10% FBS, and cells are subjected to flow cytometry 72 hours post transduction. The effect of the two adapters on transduction efficacy is evaluated essentially as described in Example 7.

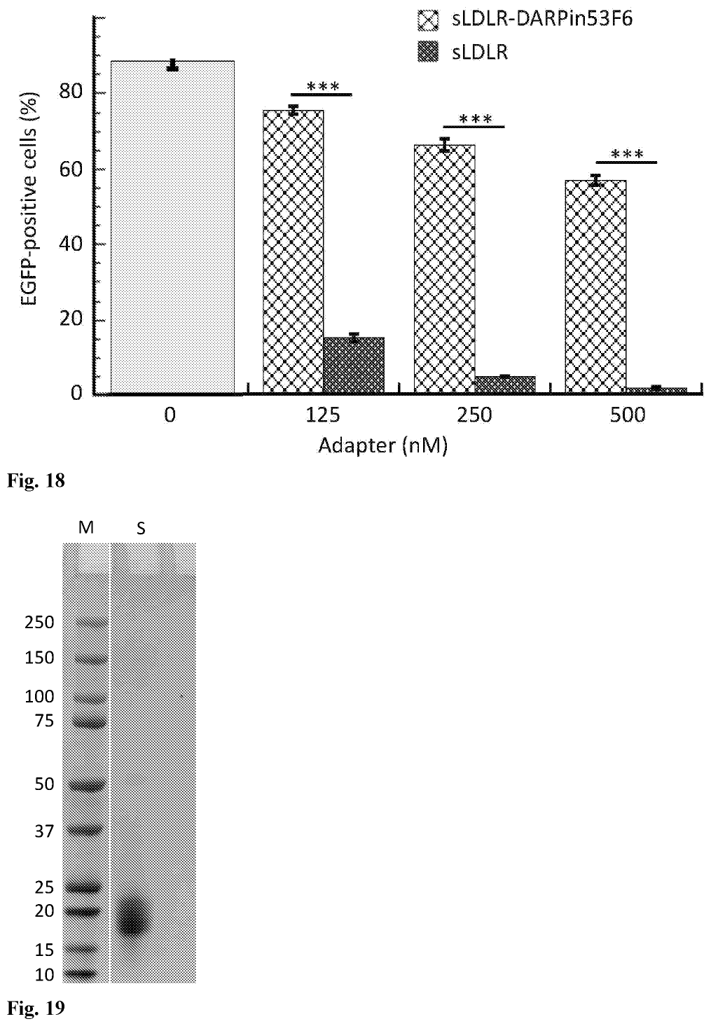

In the experiment depicted in FIG. 18. LVV (75,000 TU/mL) was pre-incubated (15 min) with the indicated concentrations of sLDLR or sLDLR-DARpin53F6. It was then added to a culture of MOLT4 cells (200,000 cells/mL; All concentrations are final) and the culture was incubated at 37° C. for 1 h in a serum-free media. The media was then replaced by media containing 20% FBS and incubation continued for 72 h. The cells were then washed, treated with diethyl pyrocarbonate (50 μL per 1 mL of cell suspension. 5 min.) and % EGFP-expressing cells was then measured by flow cytometry.

The results are presented in FIG. 18, in which “Adapter (nM)” represents the concentration of adapter molecule and sLDLR during the pre-incubation stage, and the percent of EGFP-positive MOLT4 cells is determined by flow cytometry. n=3. ***p<0.0001. As can be seen, sLDLR₂₅₋₁₈₇ effectively inhibited the transduction of the CD8-positive MOLT4 cells, whereas sLDLR-DARPin53F6 allowed transduction of the MOLT4 cells. In comparison, both adapters were equally effective in neutralizing the cytopathic effect of VSV on cells lacking CD8 expression, as determined from FIG. 17. Accordingly, the results demonstrate that sLDLR-DARPin53F6 facilitates transduction by VSV-G-decorated LVVs in a CD8-specific manner, with remarkable potency and target selectivity.

Similar examples are performed to assess the effect of the adapters on human peripheral blood mononuclear cells (PBMC). To this end, eGFP-encoding lentiviral particles (10⁶ TU/ml) are diluted 1:2 and pre-incubated with serially twofold diluted sLDLR or sLDLR-ug53F6 as described above. Human PBMC (4×10⁵ cells/well, in 70 μl Opti-MEM™) are seeded into 96-well plates. Aliquots (30 μl) of the LVV with sLDLR or sLDLR-53F6 are added to the cells and the plate is incubated for 4 h at 37° C. 5% CO₂. Then, 100 μl of DMEM supplemented with 20% fetal bovine serum is added to the cells and incubation continues for 24 h. Media is then replaced, and 72 hours post transduction cells are stained with an anti-human CD8 antibody, washed with PBS and fixed with PBS containing 1% formaldehyde. The cells are then subjected to flow cytometry and transduction efficacy is evaluated by measuring eGFP fluorescence in CD8⁺ cells and control cells.

### Example 17. Rapid In Vitro Generation of CAR-T Cells Using sLDLR-53F6

Human Peripheral blood mononuclear cells (PBMC) are obtained by leukapheresis from a patient in need. PBMC are then isolated by a density gradient and monocytes are removed by size-based method according to standard procedure. CD8⁺ cells are then isolated by Miltenyi beads, according to the manufacturer's instructions. The CD8⁺ T cells are then resuspended (2×10⁷ cells/ml) in X-VIVO 15 medium supplemented with 5% human AB serum, 2 mM L-glutamine, 20 mM HEPES, and IL-7 and IL-15 (10 ng per mL, each). Lentiviral particles encoding anti-CD19-BBζ CAR (Milone, M C. et al, 2009, Molec. Therapy, 17, 1453-1464, 1-2×10⁸ TU/ml) are mixed with sLDLR-53F6 (100 μg/mL) in the above-mentioned medium. This suspension is added to equal volume of the CD8⁺ T-cell culture (multiplicity of infection=5-10) and incubation continued for 24 h. The cells are then isolated, washed, suspended in saline and transfused to the patient.

### Example 18. In Vivo Transduction of Human PBMC Using sLDLR-53F6

6-week-old NSG mice (Charles River) are intravenously (i.v.) injected with 5×10⁶ human PBMC, followed by an i.v. injection of eGFP-encoding lentiviral particles described in Example 16 (10⁶ TU/ml, 0.2 mL) pre-mixed with either sLDLR or sLDLR-53F6 (10 μg/mL). Seven days post-vector application, blood is taken before sacrifice of the animals, the spleen is removed, and single cell suspensions are prepared by meshing the spleens through a 45-um cell strainer. Single-cell suspensions from blood and spleens are stained with anti-human CD8, subjected to erythrocyte lysis by using PharmLyse buffer (BD Biosciences), and analyzed by flow cytometry. The ability of the adapters to facilitate transduction of the LVVs to human PBMC-derived cells in vivo in the murine model is evaluated and compared.

In vivo experiments are also performed in human subjects, as follows. Human proprotein convertase subtilisin/kexin type-9 (PCSK9, Sigma, 10 mg in 500 mL saline) is intravenously injected to a patient at a rate of 5 mL/min. After 75 min, lentiviral particles encoding anti-CD19-BBζ CAR as described in Example 17, 10⁹ TU/ml, 10 mL), pre-mixed with an excess of sLDLR-53F6 (100 μg/mL), are administered IV as a bolus injection. Expression of anti-CD19-BB& CAR in CD8⁺ T cells is observed after 7-30 days.

### Example 19. Construction and Production of a Mammalian Expression Vector Encoding the PSMA-Specific Adapter GTI-sLDLR

A nucleic acid construct encoding an isolated soluble form of LDLR fused to a targeting moiety derived from a peptide ligand selective to human PSMA was designed and manufactured. This construct contains a coding sequence of the PSMA ligand GTIQPYPFSWGY (GTI, SEQ ID NO: 37), a flexible linker (GSGGGGSGG, SEQ ID NO: 24), mature human pro-LDLR₍₂₅₋₁₄₅₎ (SEQ ID NO: 5), and the His Tag in pcDNA3.1 vector. The amino acid sequences of the mature fusion protein, also referred to herein as “GTI-sLDLR” or “GTI-sLDLR₍₂₅₋₁₄₅₎”, is set forth in SEQ ID NO: 40, as follows:

(GTI-SLDLR, SEQ ID NO: 40)
GTIQPYPFSWGYGSGGGGSGGDRCERNEFQCQDGKCISYKWVCDGSAECQDGSDESQETCLSVT
CKSGDFSCGGRVNRCIPQFWRCDGQVDCDNGSDEQGCPPKTCSQDEFRCHDGKCISRQFVCDSD
RDCLDGSDEASCPVGTKHHHHHH.

The corresponding nucleic acid construct encoding pro-GTI-sLDLR (including a signal peptide at positions 1-54) for use in mammalian cell expression is set forth in SEQ ID NO: 42, as follows:

(pro-GTI-sLDLR construct, SEQ ID NO: 42)
ATGGGCTGGTCCTGTATCATCCTGTTCCTGGTGGCCACCGCTACAGGCGTGCACGGCACCATCC
AGCCTTACCCCTTCAGCTGGGGCTACGGCAGCGGCGGCGGAGGATCTGGCGGTGACAGATGCGA
GCGGAACGAGTTTCAGTGCCAGGATGGAAAATGCATCAGCTACAAGTGGGTGTGCGACGGAAGC
GCCGAGTGCCAGGACGGCTCCGATGAATCTCAAGAGACATGTCTGTCTGTCACCTGCAAGTCCG
GCGACTTCTCCTGCGGCGGCAGAGTGAATCGGTGCATCCCTCAGTTTTGGCGGTGCGATGGCCA
GGTGGACTGCGACAACGGCTCTGACGAACAGGGCTGCCCTCCAAAGACCTGTTCTCAGGACGAA
TTCAGATGTCACGACGGCAAGTGCATCTCCAGACAGTTCTGCGACAGCGATAGAGATTGCCTGG
ACGGCAGCGACGAGGCCAGCTGTCCTGTGGGCACCAAGCACCACCACCACCACCATTGA.

GTI-sLDLR was produced in HEK293T cells transfected with pcDNA3-GTI-sLDLR essentially as described in Example 2. It was then purified essentially as described in Example 2. It was then purified essentially as described in Example 2. The final volume of the eluate was 10 mL. Calculated molecular weight: 16493. Calculated absorbance of 1 mg/mL solution at 280 nm is 1.34. Concentration of Batch #30284 is 0.85 mg/mL. The results are presented in FIG. 19, in which “M” indicates the molecular weight marker, and “S” indicates the presence of the sample. As can be seen in FIG. 19, SDS-PAGE gave a single band of molecular mass 16-20 kDa, having a broad appearance characteristic of glycoproteins.

### Example 20. SLDLR and GTI-sLDLR Effectively Neutralize the Cytopathic Effect of VSV in Prostate Cancer PC3 Cells Lacking PSMA Expression

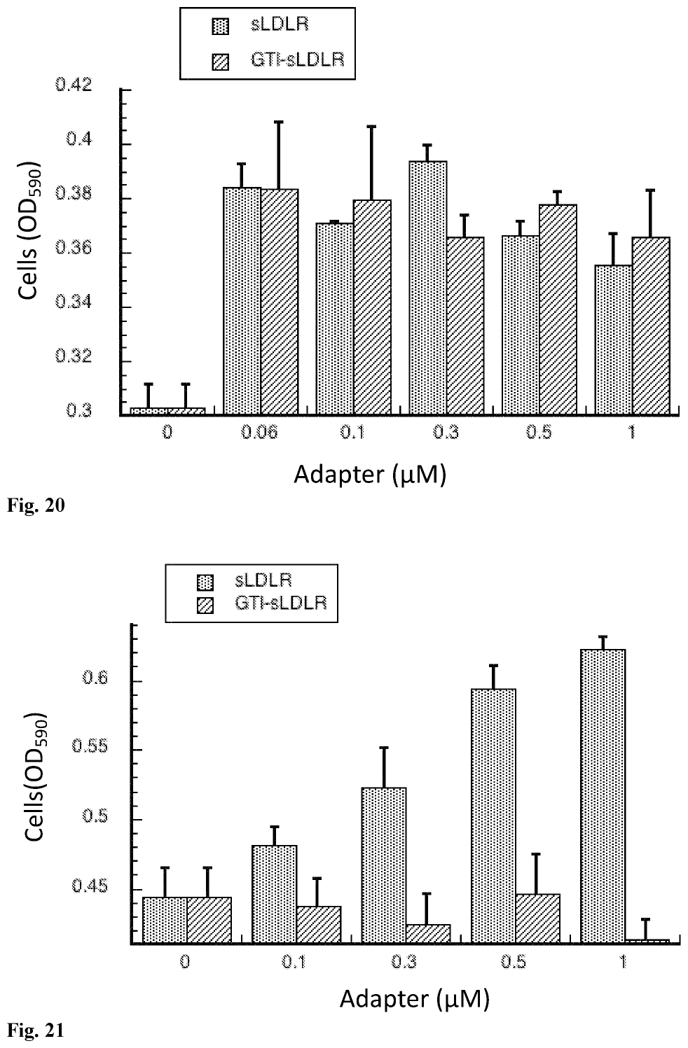

Human PC3 prostate cancer cells (4×10⁵/mL, in 0.1 mL of DMEM-10) were seeded in 96-well plates and let attach for 2 h (2 h at 37° C. in 5% CO₂). VSV was diluted separately in DMEM-10 to a concentration of 2.5×10⁵ pfu/mL, and aliquots of 0.1 mL in 96 well tissue culture plates were pre-incubated for 15 min at room temperature with different concentrations of sLDLR and GTI-sLDLR, as indicated at FIG. 20. The VSV aliquots were then added to the confluent cultures of PC3 cells and the cultures were further incubated at 37° C./5% CO₂ for 24 h. The media were then discarded, the cells were fixed for 15 min with cold methanol and then stained with 5% w/v crystal violet in 66% v/v aqueous methanol. The cells were then fixed, stained, washed, and analyzed as described in Example 4. The results are presented in FIG. 20. “Cells (OD₅₉₀)” reflects the relative number of viable cells determined by staining with Crystal violet and “Adapter (μM)” represents the concentration of adapter molecules during the pre-incubation stage. As can be seen in FIG. 20, both sLDLR₂₅₋₁₈₇ and GTI-sLDLR were effective in neutralizing the cytopathic effect of VSV on PSMA-negative PC3 cells.

### Example 21. Selective Lysis of PSMA-Positive Prostate Cancer LNCaP Cells by VSV in the Presence of GTI-sLDLR

LNCaP cells (PSMA-expressing prostate cancer cells, 8×10⁵/mL, in 0.1 mL of RPMI supplemented with penicillin 100 IU/mL, streptomycin 0.1 mg/mL; glutamine 2 mM, and 10% fetal bovine serum, hereinafter termed “RPMI-10”) were seeded in tissue culture treated 96-well plates and let attach for 2h (2 h at 37° C. in 5% CO₂). VSV was diluted separately in RPMI-10 to a concentration of 2000 pfu/mL, and aliquots of 0.1 mL in 96 well plates were pre-incubated for 15 min at room temperature with various concentrations of GTI-sLDLR or sLDLR, as indicated at FIG. 21. The VSV aliquots were then added to the cultures of LNCaP cells and the cultures were further incubated at 37° C./5% CO₂ for 24 h. The cells were then fixed, stained, washed, and analyzed as described in Example 4. The results and are presented in FIG. 21. As can be seen, in LNCaP cells, sLDLR significantly neutralized the cytopathic effect of VSV, as in PC3 cells. In contradistinction, the cytopathic effect of VSV on PSMA-expressing LNCaP cells was substantially retained even in the presence of GTI-sLDLR. In contradistinction, as evident from FIG. 20, the cytopathic effect of VSV on PC3 cells lacking PSMA was substantially neutralized in the presence of GTI-sLDLR. Thus. GTI-sLDLR was capable of mediating VSV-induced oncolysis in PSMA-expressing prostate cancer target cells, while inhibiting oncolysis in PSMA-negative cells, demonstrating remarkable potency and target selectivity.

### Example 22. Preparation of Adapters Comprising Folic Acid Conjugated to sLDLR

For the conjugation of folic acid (FA) to sLDLR via a flexible poly(oxyethylene) (PEG) linker, commercially available 2-Chlorotrityl Fmoc-Cys-S-TRT, polymer-bound (04231 Chemimpex; 30 mg. 1 equiv., 0.051 mmol) is swollen with dichloromethane (3 mL) for 15 min followed by 2-propanol (2×3 mL) for 5 min each time and DMF (2×3 mL) for 10 min each. Then, a solution of 20% piperidine in DMF (3×3 mL) is added to the resin. Then, the resin is washed with DMF and 2-propanol, and confirmed deprotection by a Kaiser test. After swelling the resin in DMF, a solution of Fmoc-glu-(OtBu)-OH (54.3 mg. 2.5 equiv., 0.13 mmol). PyBOP (66.3 mg. 2.5 equiv., 0.13 mmol) and diisopropylethylamine (DIPEA. 0.089 mL. 10 equiv., 0.51 mmol) in DMF (1.5 mL) is added to the resin and bubbled under nitrogen for 4 h. Then, the resin is again washed with DMF (2×3 mL) for 10 min and 2-propanol (2×3 mL) for 5 min and tested by a Kaiser test. After confirming the coupling of glutamic acid on the resin. 20% piperidine in DMF (3×3 mL) is added to the resin. Then, the resin is washed with DMF. 2-propanol and confirmed deprotection by a Kaiser test. After swelling the resin in DMF, a solution of N10-(trifluoroacetyl) pteroic acid (861545 Sigma; 31.2 mg, 1.5 equiv., 0.0765 mmol), PyBOP (66.3 mg. 2.5 equiv., 0.13 mmol) and DIPEA (0.089 mL. 10 equiv., 0.51 mml) in DMF (1.5 mL) is added to the resin and bubbled with dry nitrogen overnight. Then, the resin is washed with DMF (2×3 mL) for 10 min and 2-propanol (2×3 mL) for 5 min. After performing a Kaiser test, the final compound is cleaved from the resin using the cocktail solution (TFA: H2O: triisopropylsilane; 95:2.5:2.5) (3×3 mL) for 30 min each time. The compound is concentrated under vacuum. Excess TFA is evaporated with rotary evaporator. Then, the crude product is dissolved in DMSO, precipitated with excess cold diethyl ether and centrifuged at 2000 rpm for 10 min. The supernatant is removed. The product is dissolved in dimethyl sulfoxide (DMSO) and purified on Prep. RP-HPLC using a gradient mobile phase of A=20 mM ammonium acetate buffer (pH=7) and B=acetonitrile; using a gradient from 0% B to 50% B over 35 min (column: Waters xTerra C18. 10 μm; 19×250 mm). Elution of the folate-containing product is monitored at Δ=280 nm and 360 nm and the identities of the eluted compounds were analyzed by LC-MS. Then, the tert-butyl ester which protected carboxyl group on glutamic acid is removed by using hydrogen chloride solution (4.0 M in dioxane) for 1 h at room temp. Finally, the N10-trifluoroacetic acid group is deprotected by using mild 0.5 M aq. ammonium hydroxide (NH₄OH) buffer for 2 h at room temperature. The final compound, folic acid-CONH-cysteine (FA-CONH-Cys) is purified again by using RP-HPLC in the same condition as above. The compound is dried by a rotary evaporator (or by lyophilization), redissolved in water and dried again. This step is repeated several times until no ammonium acetate is detectable in the final product. It is then dried, redissolved in a minimal volume of dry DMSO and stored in the dark at −20° C. The identity of FA-CONH-Cys and its exact concentration is determined by LC-MS and amino acid analysis. DMSO solution of FA-CONH-Cys (4 equivalents), DIPEA (4 equivalents) and Mal-amido-PEGs-CO—NHS (Cat. #BP-22159, BroadPharm; 4 equivalents) in dried DMSO are mixed at and left for 4 h at room temp. The solution is then added to an aqueous solution of sLDLR₍₂₅₋₁₄₅₎ (1 equivalent at pH 7.5) at 4° C. The solution is left for 4-16 h in the dark and then resolved by Superdex 75 size exclusion chromatography column using HEPES Buffer, 10 mM, pH 7.4, NaCl 140 mM and CaCl₂ 0.2 g/L and monitoring at 280/370 nM. The conjugation product is kept at −80° C. in the dark. The resulting adapter, containing FA-PEGs-conjugated sLDLR₍₂₅₋₁₄₅₎ molecules, is herein designated FA-PEG-sLDLR.

For the conjugation of FA directly to sLRLR, a solution of FA-OSu (Synchem UG, Germany) in anhydrous dimethyl sulfoxide (70 mg/mL, 124 μM, 2 μL) was added to an aqueous solution of sLDLR₍₂₅₋₁₈₇₎ (SEQ ID NO: 7. 13.1 μM. 1 mL) and the mixture was left for 18 h in the dark at 4° C. with occasional shaking. Glycine, 10 mg/mL, 5 μL, was added and after 15 min. the mixture was spun 12,000×g 1 min. The supernatant was ultra-filtered using Amicon Ultra-4 10K centrifugal filter to 0.1 mL, diluted with 0.4 mL HEPES buffer pH 7.4, 10 mM, NaCl 140 mM, containing CaCl₂, (0.2 g/L) and ultra-filtered again to 0.1 mL. This procedure was repeated several times until the OD₃₆₅ of the effluent was below 0.06. The upper phase containing the conjugation product was diluted to 0.5 mL, and aliquots of 50 μL were stored at −80° C. in the dark. The resulting adapter, containing FA-conjugated sLDLR₍₂₅₋₁₈₇₎ molecules, is herein designated FA-sLDLR.

### Example 23. Transduction of Cells Expressing Folate Receptor (FOLR1) Using FOLR1-Specific Adapter

eGFP-encoding LVV (VectorBuilder; 10⁶ TU/ml) is diluted 1:2 in either Opti-MEM™ alone, or in Opti-MEM™ containing FA (10 μg/mL). These LVV suspensions are pre-incubated for 15 min at room temperature with serially twofold diluted adapter (FA-PEGs-sLDLR or FA-sLDLR, starting at 10 μg/mL; or, in other experiments, 40 μL). Human (folate receptor 1-positive) HeLa cells (5×10⁵ cells/mL in DMEM-10) are seeded in 96-well plates (0.1 mL/well) and incubated at 37° C./5% CO₂ for 4 h. The culture medium of the Hela cells in 96 well plate is removed and replaced by the vector-adapter mixtures (with or without free FA, 30 L/well). The cultures are incubated for 4 h. DMEM-10 (0.17 mL/well) is then added, and the plates are incubated for additional 24 h. All media is then replaced with fresh DMEM-10, 0.2 mL and the cells are then grown for additional 48 h. The plates are washed with PBS and fluorescence intensity is measured using a fluorescence plate reader. Specific transduction, namely transduction mediated by folate receptor 1 (FOLR1) expressed on the target cells rather than by LDLR, is evaluated by measuring fluorescence intensity in the presence or absence of free FA (used as a competitive inhibitor); significant fluorescence in the absence of free FA, and completely or substantially non-detectable fluorescence in the presence of free FA, indicates a FOLR1-specific transduction.

### Example 24. In Vitro Transduction of Hematopoietic Stem Cells Using FOLR1-Specific Adapter

eGFP-encoding lentiviral particles (VectorBuilder; 10⁶ TU/ml) are diluted 1:2 in either Opti-MEM™, or FA in Opti-MEM™ (10 μg/mL) and pre-incubated for 15 min at room temperature with serially twofold diluted adapter (FA-PEGs-sLDLR or the FA-sLDLR control, starting at 10 μg/mL). Human CD34⁺ hematopoietic stem cells (HSC) obtained from G-CSF-treated (10 μg/Kg/day. 4 days) donors (Lonza; 4×10⁵ cells/well, in 70 μl Opti-MEM™) are seeded into 96-well plates. Aliquots (30 μl) of the LVV with FA-PEGs-sLDLR or FA-sLDLR are added to the cells, in the presence or absence of free FA, and the plate is incubated for 4 h at 37° C. 5% CO₂. Then, 100 μl of DMEM supplemented with 20% fetal bovine serum is added to the cells and incubation continues for 24 h. Media is then replaced, and 72 hours post transduction cells are washed with PBS and fixed with PBS containing 1% formaldehyde. The same study is performed on healthy donor PBMC (obtained without G-CSF mobilization) as a negative control. The cells are then subjected to flow cytometry and transduction efficacy is evaluated by measuring eGFP fluorescence in CD34⁺ HSC and in the PBMC control cells, essentially as described in Example 23. Specific transduction in the CD34⁺ HSC (which are also FOLR1⁺), and no substantial transduction in the presence of free FA and in the PBMC control cells (which are mostly FOLR1⁻), indicates the applicability of the adapter to mediate the transduction of gene therapy agents into HSC using LVV vectors.

### Example 25. In Vivo Transduction of Human CD34⁺ Hematopoietic Stem Cells Using FOLR1-Specific Adapter

6-week-old NSG mice (Charles River) are intravenously (i.v.) injected with 5×10⁶ human HSC obtained from G-CSF-treated donors (Lonza), followed by an i.v. injection of eGFP-encoding lentiviral particles described in Example 24 (10⁶ TU/ml, 0.2 mL) pre-mixed with either sLDLR or the FOLR1-specific adapter (FA-PEGs-sLDLR or FA-sLDLR, 10 μg/mL). Seven days post-vector application, the mice are injected subcutaneously twice daily with human G-CSF (125 μg/kg) for 5 days. Blood is taken before sacrifice of the animals and PBMC are isolated. The spleen is removed, and single cell suspensions are prepared by meshing the spleens through a 45-μm cell strainer. PBMC and single-cell suspensions from the spleens are stained with anti-human CD34⁺ and analyzed by flow cytometry for CD34⁺ and for GFP. The ability of the adapters to facilitate transduction of the LVVs to human HSC-derived cells in vivo in the murine model is evaluated and compared. In vivo treatments are also performed in human patients, as follows. Human PCSK9 (Sigma, 10 mg in 500 mL saline) is intravenously injected to a patient at a rate of 5 mL/min. After 75 min, lentiviral particles encoding a desired gene, 10⁹ TU/ml, 10 mL), pre-mixed with an excess of the adapter (FA-PEGs-sLDLR or the FA-sLDLR control, 100 μg/mL), are administered IV as a bolus injection. Expression of the desired gene in PBMC is observed after 7-30 days.

In summary, disclosed herein is the construction of adapter molecules, nucleic acid constructs encoding them, and viruses and viral vectors comprising these adapters and constructs. Further demonstrated herein is the use of these adapters, constructs and viral agents were demonstrated to mediate VSV-induced oncolysis, as well as gene delivery mediated by VSV-G-decorated LVV, with unexpectedly high potency and target selectivity, to various target cells including when targeting TAA such as PSMA, CEACAM6, CEACAM1, c-KIT and FOLR1, as well as cell surface receptors expressed preferentially on lymphocytes such as CD8, or on HSC, such as FOLR1. Thus, the results demonstrate the unexpected applicability of adapters utilizing isolated hLDLR CR domains, which notably lack the LDLR beta-propeller domain, in mediating highly selective and potent viral therapy.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-69. (canceled)

70. An adapter molecule, comprising an anchoring component covalently linked by a flexible linker to a targeting component, wherein:
a. the anchoring component consists essentially of at least one isolated Class A repeat (CR) motif selected from the group consisting of: human low-density lipoprotein receptor (hLDLR) Class-A repeat 2 (hLDLR CR2) and hLDLR CR3, and optionally at least one of hLDLR CR1 and hLDLR CR4,
b. the flexible linker comprises at least four contiguous amino acid residues selected from the group consisting of glycine, serine and/or alanine, and
c. the targeting component comprises a ligand of a receptor expressed preferentially on the surface of a mammalian target cell, or an antigen-binding molecule that selectively binds the receptor.

71. The adapter molecule of claim 70, wherein the anchoring component consists essentially of a plurality of the isolated CR motifs, or
wherein the anchoring component consists essentially of hLDLR CR1, hLDLR CR2 and hLDLR CR3.

72. The adapter molecule of claim 71, wherein said anchoring component is selected from the group consisting of: sLDLR₍₂₅₋₁₄₅₎ (SEQ ID NO: 5), sLDLR₂₅₋₁₄₉ (SEQ ID NO: 6) and sLDLR₂₅₋₁₈₇ (SEQ ID NO: 7).

73. The adapter molecule of claim 70, wherein said receptor is selected from the group consisting of: carcinoembryonic antigen-related cell adhesion molecule 6 (CEACAM6), cluster of differentiation 8 (CD8), CD56, prostate-specific membrane antigen (PSMA), CEACAM1, proto-oncogene c-KIT (c-KIT), and folate receptor (FOLR1).

74. The adapter molecule of claim 73,
wherein said anchoring component consists essentially of sLDLR₍₂₅₋₁₄₅₎ (SEQ ID NO: 5) and said targeting component is CEACAM8₍₃₅₋₁₄₀₎ (SEQ ID NO: 12), or
wherein said anchoring component consists essentially of sLDLR₍₂₅₋₁₄₅₎ (SEQ ID NO: 5), and said targeting component is DARPin53F6 (SEQ ID NO: 11), or
wherein said anchoring component consists essentially of sLDLR₍₂₅₋₁₄₅₎ (SEQ ID NO: 5), and said targeting component consists essentially of a GTI peptide
(GTIQPYPFSWGY, SEQ ID NO: 37).

75. A nucleic acid construct encoding the adapter molecule of claim 70, wherein said adapter molecule is a fusion protein consisting essentially of said anchoring component, said targeting component and said linker.

76. A viral vector comprising the nucleic acid construct of claim 75, wherein said construct is operably linked to one or more transcription regulation sequences,
the viral vector optionally being selected from the group consisting of recombinant vesicular stomatitis virus (VSV), Cocal virus (COV), and Maraba virus (Maraba) vectors.

77. The adapter molecule of claim 70, which is specifically complexed in a non-covalent manner with particles of a virus or viral vector decorated with a vesiculovirus envelope glycoprotein (G) selected from the group consisting of: VSV-G, COV-G, and Maraba-G, to form adapter-modified viral particles.

78. A pharmaceutical composition comprising a therapeutically effective amount of the adapter-modified viral particles as defined in claim 77, further comprising a pharmaceutically acceptable carrier, excipient or diluent.

79. The pharmaceutical composition of claim 78, further comprising at least one of: (i) a proprotein convertase subtilisin/kexin type-9 (PCSK9) polypeptide, and (ii) a second composition of said adapter molecule, such that the total amount of said adapter molecule in said pharmaceutical composition is in excess of said viral particles.

80. The pharmaceutical composition of claim 75, wherein said viral vector further encodes a chimeric antigen receptor (CAR), a gene therapy agent or a gene editing agent.

81. The pharmaceutical composition of claim 80, wherein said gene therapy agent is a human CFTR (hCFTR) gene product, said viral vector is a lentiviral or retroviral vector pseudotyped with said envelope glycoprotein, and the targeting component of said adapter molecule comprises a CEACAM6- or CEACAM1-binding portion of human CEACAM8 (hCEACAM8), an antigen-binding portion of an antibody directed to human CEACAM6 (hCEACAM6) or human CEACAM1 (hCEACAM1), or a CEACAM1-binding portion of human CEACAM5 (hCEACAM5), or
wherein the targeting component comprises an antigen-binding molecule that selectively binds to human CD8 or CD56, and said viral vector is a lentiviral or retroviral vector pseudotyped with said envelope glycoprotein, and encoding a CAR directed to a TAA, or
wherein said adapter molecule is characterized in that said anchoring component consists essentially of sLDLR₍₂₅₋₁₄₅₎ (SEQ ID NO: 5), and said targeting component is CEACAM8₍₃₅₋₁₄₀₎ (SEQ ID NO: 12).

82. The pharmaceutical composition of claim 80, wherein said virus is an oncolytic virus further encoding said adapter molecule.

83. The pharmaceutical composition of claim 82, wherein the oncolytic virus is a vesiculovirus encoding said envelope glycoprotein, and the targeting component of said adapter molecule is directed to a TAA optionally selected from the group consisting of hCEACAM6, hCEACAM1, human c-KIT, and human PSMA.

84. A process for producing the pharmaceutical composition of claim 78, comprising contacting the particles of the virus or viral vector of (ii) with adapter molecule of (i), so as to produce the adapter-modified viral particles.

85. A method of delivering a virus or viral vector selectively into a target cell in a subject in need thereof, comprising contacting particles of the virus or viral vector with the adapter molecule of claim 70, so as to produce adapter-modified viral particles, and administering the resulting adapter-modified viral particles to the subject.

86. The method of claim 85, wherein said virus is selected from the group consisting of VSV, COV and Maraba viruses, or wherein said viral vector is selected from the group consisting of vesiculoviral and lentiviral vectors.

87. The method of claim 86, wherein said target cell is selected from the group consisting of: a tumor cell, an immune cell, a hematopoietic stem cell (HSC), and a lung epithelial cell.

88. The method of claim 87, wherein:
a) said target cell is a lung epithelial cell and said receptor is CEACAM6 or CEACAM1;
b) said target cell is a tumor cell and said receptor is selected from the group consisting of: PSMA, c-KIT, FOLR1, CEACAM6 and CEACAM1;
c) said target cell is an immune cell and said receptor is CD8 or CD56; or
d) said target cell is a HSC and said receptor is FOLR1.

89. The method of claim 88, wherein:
a) said target cell is a lung epithelial cell, said targeting component of said adapter is CEACAM8₍₃₅₋₁₄₀₎ (SEQ ID NO: 12), and said viral vector is a VSV-G pseudotyped lentiviral or retroviral vector encoding a CFTR gene product; or
b) said target cell is a PSMA⁺ tumor cell, said targeting component of said adapter is a selective PSMA ligand (PSMAL) comprising a Glu-NH—CO—NH-Lys pharmacophore or a GTI peptide (GTIQPYPFSWGY, SEQ ID NO: 37), and said virus or viral vector is an oncolytic vesiculovirus or a vesiculoviral vector further encoding said adapter molecule;
c) said target cell is a CD8⁺ immune cell, said targeting component of said adapter is DARPin53F6 (SEQ ID NO: 11), and said viral vector is a VSV-G pseudotyped lentiviral or retroviral vector encoding a CAR directed to a TAA;
d) said target cell is a HSC, said targeting component of said adapter is folic acid, and said viral vector is a VSV-G pseudotyped lentiviral or retroviral vector encoding a gene therapy agent, or
wherein said anchoring component is selected from the group consisting of: sLDLR₍₂₅₋₁₄₅₎ (SEQ ID NO: 5), sLDLR₂₅₋₁₄₉ (SEQ ID NO: 6) and sLDLR₂₅₋₁₈₇ (SEQ ID NO: 7).

90. A method of treating a disease or condition in a subject in need thereof, comprising administering to the subject the pharmaceutical composition of claim 78.

91. The method of claim 90, wherein the disease or condition is an inherited monogenic disorder, and said composition comprises adapter-modified particles of a viral vector encoding a gene therapy agent.

92. The method of claim 91, wherein the disorder is cystic fibrosis, the gene therapy agent is a human CFTR (hCFTR) gene product, and wherein said viral vector is a lentiviral or retroviral vector pseudotyped with said envelope glycoprotein and the targeting component of said adapter molecule comprises a receptor-binding portion of hCEACAM8, a receptor-binding portion of hCEACAM5, or an antigen-binding portion of an antibody directed to hCEACAM6, or
wherein said anchoring component is selected from the group consisting of: sLDLR₍₂₅₋₁₄₅₎ (SEQ ID NO: 5), sLDLR₂₅₋₁₄₉ (SEQ ID NO: 6) and sLDLR₂₅₋₁₈₇ (SEQ ID NO: 7), or
wherein said adapter molecule is characterized in that said anchoring component consists essentially of sLDLR₍₂₅₋₁₄₅₎ (SEQ ID NO: 5), and said targeting component is CEACAM8₍₃₅₋₁₄₀₎ (SEQ ID NO: 12).

93. The method of claim 90, wherein the disease or condition is a tumor,
wherein said tumor is selected from the group consisting of a hematological tumor, a lung tumor, a prostate tumor, a breast tumor, a gynecological tumor, a pancreatic tumor and malignant glioma, or
wherein said virus is an oncolytic virus further encoding said adapter molecule, or
wherein said adapter molecule is characterized in that said anchoring component consists essentially of sLDLR₍₂₅₋₁₄₅₎ (SEQ ID NO: 5), and said targeting component is CEACAM8₍₃₅₋₁₄₀₎ (SEQ ID NO: 12).
